(19) United States
(12) Patent Application Publication
(10) Pub. No.: US 2007/0270357 A1
(43) Pub. Date: Nov. 22, 2007

(54) Bifunctional Macrolide Heterocyclic Compounds and Methods of Making and Using the Same

(76) Inventors: Jay J. Farmer, Codi, NY (US); Joel A. Goldberg, Philadelphia, PA (US); Adegboyega K. Oyelere, Marietta, GA (US); Joseph M. Salvino, Chester Springs, PA (US); Dane M. Springer, Yardley, PA (US); Jennifer Tran, Guilford, CT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/579,706

(22) PCT Filed: Nov. 18, 2004
(86) PCT No.: PCT/US04/38777

§ 371 Date: Mar. 12, 2007

Related U.S. Application Data
(60) Provisional application No. 60/523,207, filed on Nov. 18, 2003.

(51) Int. Cl. A61K 31/7052; A61F 2/06; A61P 31/04; C07H 17/08; C07H 17/00; A61K 31/7048

(52) U.S. Cl. 514/28; 536/7.4; 623/1.42

(57) ABSTRACT

The present invention relates generally to the field of anti-infective, anti-proliferative, anti-inflammatory, and prokinetic agents. More particularly, the invention relates to a family of bifunctional compounds useful as therapeutic agents. These compounds have both a macrolide ring and at least one heterocyclic moiety. The present invention further relates to processes for the preparation of such compounds, to intermediates useful in their preparation, to the use of the compounds as therapeutic agents, and to pharmaceuticals compositions containing them.

### RELATED APPLICATION

This application claims the benefit of and priority to U.S. patent application Ser. No. 60/523,207, filed Nov. 18, 2003, the disclosure of which is incorporated by reference herein.

### FIELD OF THE INVENTION

The present invention relates generally to the field of anti-infective, anti-proliferative, anti-inflammatory, and prokinetic agents, and more particularly, the invention relates to a family of bifunctional macrolide heterocyclic compounds useful as such agents.

### BACKGROUND

Since the discovery of penicillin in the 1920s and streptomycin in the 1940s, many new compounds have been discovered or specifically designed for use as antibiotic agents. It was once believed that infectious diseases could be completely controlled or eradicated with the use of such therapeutic agents. However, such beliefs have been challenged by the fact that strains of microorganisms resistant to currently effective therapeutic agents continue to evolve. Almost every antibiotic agent developed for clinical use has encountered problems with the emergence of resistant bacteria For example, resistant strains of Gram-positive bacteria such as methicillin-resistant staphylocci, penicillin-resistant streptococci, and vancomycin-resistant enterococci have developed, and can cause serious and often time fatal results for patients infected with such resistant bacteria. Bacteria that are resistant to the macrolide antibiotics have developed. Also, Gram-negative strains of bacteria such as H. influenzae and M. catarrhalis have been identified. See, e.g., F. D. Lowry, Antimicrobial resistance: the example of Staphylococcus aureus, J. Clin. Invest., vol. 111, no. 9, pp. 1265-1273 (2003); and Gold, H. S. and Moellering, R. C., Jr., Antimicrobial-drug resistance. N. Engl. J. Med., vol. 335, pp. 1445-53 (1996).

This problem of resistance is not limited to the area of anti-infective agents, because resistance has also been encountered with anti-proliferative agents used in cancer chemotherapy. Therefore, the need exists to develop new anti-infective and anti-proliferative agents that are both effective against resistant bacteria and strains of cells and against which bacteria and strains of cells are less likely to develop resistance.

Despite this problem of increasing antibiotic resistance, no new major classes of antibiotics have been developed for clinical use since the approval in the United States in 2000 of the oxazolidinone ring-containing antibiotic, N-[[(5S)-3-[3-fluoro-4-(4-morpholinyl)phenyl]-2-oxo-5-oxazolidinyl]methyl acetamide (see structure A), which is known as linezolid and which is sold under the tradename Zyvox®. See, R. C. Moellering, Jr., Linezolid: The First Oxazolidinone Antimicrobial, Annals of Internal Medicine, vol. 138, no. 2, pp. 135-142 (2003).

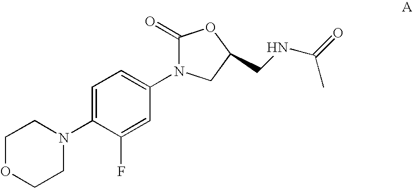

Linezolid was approved for use as an anti-bacterial agent active against Gram-positive organisms. However, linezolid-resistant strains of organisms are already being reported. See Tsiodras et al., Lancet, vol. 358, p. 207 (2001); Gonzales et al., Lancet, vol. 357, p. 1179 (2001); Zurenko et al., Proceedings Of The 39^{th} Annual Interscience Conference On Antibacterial Agents And Chemotherapy (ICAAC), San Francisco, Calif., USA (Sep. 26-29, 1999). However, investigators have been working to develop other effective linezolid derivatives. Research has indicated that the oxazolidinone ring could be important for linezolid's activity. The literature describes molecules having small groups substituted at the C-5 of the oxazolidinone ring, and early structure-activity relationships suggested that compounds with larger groups at the C-5 position were less active as anti-bacterial agents. As a consequence, investigators have been reluctant to place large substituents at the C-5 position of oxazolidinone rings in developing new anti-microbial agents.

Another class of antibiotics is the macrolides, which is so named for the 14- to 16-membered ring that is the major structural characteristic of this class of compounds. The first macrolide antibiotic to be developed was erythromycin, which was isolated from a soil sample from the Philippines in 1952. Even though erythromycin has been one of the most widely prescribed antibiotics, it has the disadvantages of relatively low bioavailability, gastrointestinal side effects, and a limited spectrum of activity. See Yong-Ji Wu, Highlights of Semi-synthetic Developments from Erythromycin A, Current Pharm. Design, vol. 6, pp. 181-223 (2000), and Yong-Ji Wu and Wei-uo Su, Recent Developments on Ketolides and Macrolides, Curr. Med Chem, vol. 8, no. 14, pp. 1727-1758 (2001).

In the search for new therapeutic agents, pharmaceutical researchers have tried combining or linking various portions of antibiotic molecules. However, this approach has met with limited success.

U.S. Pat. No. 5,693,791, to Truett, issued Dec. 2, 1997 describes an antibiotic of the formula:
A-L—B
wherein A and B are antibiotics selected from the group consisting of sulfonamides, penicillins, cephalosporins, quinolones, chloramphenicol, erythromycin (i.e., a macrolide antibiotic), metronidzole, tetracyclines, and aminoglycosides, and L is a linker formed from a difunctional linking agent.

PCT publication No. WO 99/63937, to Advanced Medicine, Inc., published Dec. 16, 1999, describes multi-binding compounds useful as antibiotics that are of the following formula:
(L)ₚ(X)_{q}
wherein L is selected from the group consisting of a macrolide antibiotic, an aminoglycoside, lincosamide, oxazolidinone, streptogramin, tetracycline, or another compound that binds to bacterial ribosomal RNA and/or to one or more proteins involved in ribosomal protein synthesis in the bacterium. In the formula, p is an integer from 2-10, q is an integer from 1-20, and X is a linker.

U.S. Pat. No. 6,034,069, to Or et al., issued Mar. 7, 2000 depicts a series of 3′-N-modified 6-O-substituted erythromycin ketolide derivatives such as shown below, wherein R, R¹, and R² are selected from the group consisting of a variety of groups, including aryl-alkoxy-heteroaryl-alkylene, R^{p} is H or a hydroxy protecting group, W is absent or is O, NH, or NCH₃, and R^{w} is H or an optionally substituted alkyl group:

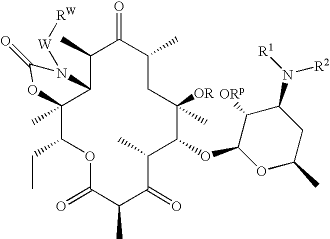

Notwithstanding the foregoing, there is an ongoing need for new anti-infective and anti-proliferative agents. Furthermore, because many anti-infective and anti-proliferative agents have utility as anti-inflammatory agents and also as prokinetic (gastrointestinal modulatory) agents, there is also an ongoing need for new compounds useful as anti-inflammatory and prokinetic agents. The present invention provides compounds which meet these needs.

### SUMMARY OF THE INVENTION

The present invention provides compounds useful as anti-infective agents and/or anti-proliferative agents, for example anti-microbial agents, anti-bacterial agents, anti-biotic agents, anti-fungal agents, anti-parasitic agents, anti-viral agents, and chemotherapeutic agents. The present invention also provides compounds useful as anti-inflammatory agents, and/or prokinetic (i.e. gastrointestinal modulatory) agents. The present invention also provides pharmaceutically acceptable salts, ester, or prodrugs thereof.

The present invention provides compounds having both a both a macrolide ring and at least one heterocyclic moiety having the formula: 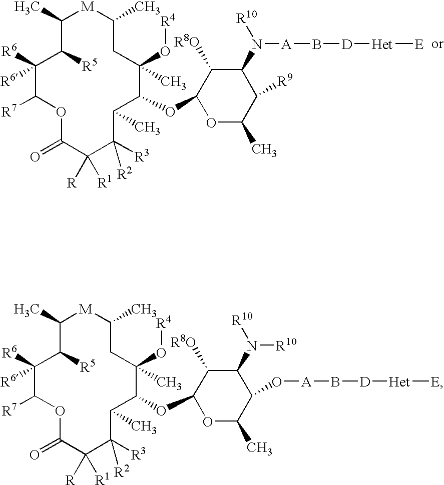
or a stereoisomer, or pharmaceutically acceptable salt, ester or prodrug thereof, wherein D-Het is selected from the group consisting of 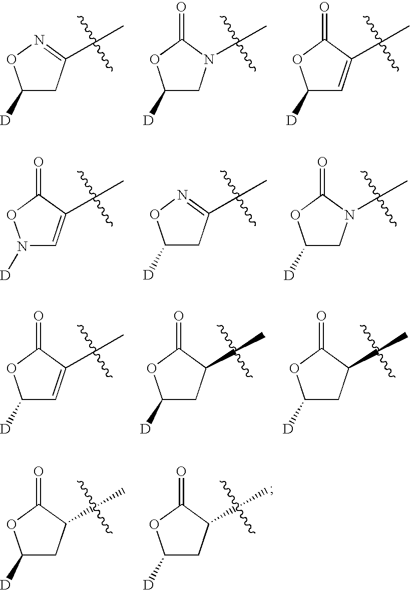
B can be selected from the group consisting of a saturated, unsaturated, or aromatic carbocycle or heterocycle, and the variables A, D, E, M, R, R¹, R², R³, R⁴, R⁵, R⁶, R^{6′}, R⁷, R⁸, R⁹, and R¹⁰ can be selected from the group consisting of the respective chemical moieties later defined in the detailed description.

In addition, the invention provides methods of synthesizing the foregoing compounds and useful chemical intermediates for synthesizing the foregoing compounds. Following synthesis, a therapeutically effective amount of one or more of the compounds can be formulated with a pharmaceutically acceptable carrier for administration to a mammal for use as an anti-cancer, anti-microbial, anti-biotic, anti-fungal, anti-parasitic or anti-viral agent, or to treat a proliferative disease, an inflammatory disease or a gastrointestinal motility disorder. Accordingly, the compounds or the formulations can be administered, for example, via oral, parenteral, or topical routes, to provide an effective amount of the compound to the mammal.

These and other aspects and embodiments of the invention can be more fully understood by reference to the following detailed description and claims.

### DETAILED DESCRIPTION OF THE INVENTION

The present invention provides a family of compounds that can be used as anti-proliferative agents and/or anti-infective agents. The compounds may be used without limitation, for example, as anti-cancer, anti-microbial, anti-bacterial, anti-fungal, anti-parasitic and/or anti-viral agents. Further, the present invention provides a family of compounds that can be used without limitation as anti-inflammatory agents, for example, for use in treating chronic inflammatory airway diseases, and/or as prokinetic agents, for example, for use in treating gastrointestinal motility disorders such as gastroesophageal reflux disease, gastroparesis (diabetic and post surgical), irritable bowel syndrome, and constipation.

The compounds described herein may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic, and geometric isomeric forms of a structure are intended, unless the specific stereochemistry or isomeric form is specifically indicated. AU processes used to prepare compounds of the present invention and intermediates made therein are considered to be part of the present invention. All tautomers of shown or described compounds are also considered to be part of the present invention.

1. Definitions

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Ring double bonds, as used herein, are double bonds that are formed between two adjacent ring atoms (e.g., C═C, C═N, or N═N).

The present invention is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

When any variable (e.g., R³) occurs more than one time in any constituent or formula for a compound, its definition at each occurrence is independent of its definition at every other occurrence. Thus, for example, if a group is shown to be substituted with one or more R³ moieties, then the group may optionally be substituted with one, two, three, four, five, or more R³ moieties, and R³ at each occurrence is selected independently from the definition of R³. Also, combinations of substituents and/or variables are permissible, but only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible, but only if such combinations result in stable compounds.

In cases wherein there are nitrogens in the compounds of the present invention, these can be converted to N-oxides by treatment with an oxidizing agent (e.g., MCPBA and/or hydrogen peroxides) to afford other compounds of the present invention. Thus, all shown and claimed nitrogens are considered to cover both the shown nitrogen and its N-oxide (N→O) derivative.

As used herein, “alkyl” is intended to include both branched and straight-chain saturated aliphatic hydrocarbon groups having the specified number of carbon atoms. C₁₋₆ alkyl is intended to include C₁, C₂, C₃, C₄, C₅, and C₆ alkyl groups. C₁₋₈ alkyl is intended to include C₁, C₂, C₃, C₄, C₅, C₆, C₇, and C₈ alkyl groups. Examples of alkyl include, but are not limited to, methyl, ethyl, n-propyl, i-propyl, n-butyl, s-butyl, t-butyl, n-pentyl, s-pentyl, n-hexyl, n-heptyl, and n-octyl.

As used herein, “alkenyl” is intended to include hydrocarbon chains of either straight or branched configuration and one or more unsaturated carbon-carbon bonds that may occur in any stable point along the chain, such as ethenyl and propenyl. C₂₋₆ alkenyl is intended to include C₂, C₃, C₄, C₅, and C₆ alkenyl groups. C₂₋₈ alkenyl is intended to include C₂, C₃, C₄, C₅, C₆, C₇, and C₈ alkenyl groups.

As used herein, “alkynyl” is intended to include hydrocarbon chains of either straight or branched configuration and one or more triple carbon-carbon bonds that may occur in any stable point along the chain, such as ethynyl and propynyl. C₂₋₆ alkynyl is intended to include C₂, C₃, C₄, C₅, and C₆ alkynyl groups. C₂₋₈ alkynyl is intended to include C₂, C₃, C₄, C₅, C₆, C₇, and C₈ alkynyl groups.

Furthermore, “alkenyl”, and “alkynyl” are intended to include moieties which are diradicals, i.e., having two points of attachment, an example of which in the present invention is when L is selected from these chemical groups. A nonlimiting example of such an alkyl moiety that is a diradical is —CH₂CH₂—, i.e., a C₂ alkyl group that is covalently bonded via each terminal carbon atom to the remainder of the molecule.

As used herein, “cycloalkyl” is intended to include saturated ring groups, such as cyclopropyl, cyclobutyl, or cyclopentyl. C₃₋₈ cycloalkyl is intended to include C₃, C₄, C₅, C₆, C₇, and C₈ cycloalkyl groups.

As used herein, “halo” or “halogen” refers to fluoro, chloro, bromo, and iodo. “Counterion” is used to represent a small, negatively charged species such as chloride, bromide, hydroxide, acetate, and sulfate.

As used herein, “haloalkyl” is intended to include both branched and straight-chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include, but are not limited to, trifluoromethyl, trichloromethyl, pentafluoroethyl, and pentachloroethyl.

As used herein, “alkoxy” refers to an alkyl group as defined above with the indicated number of carbon atoms attached through an oxygen bridge. C₁₋₆ alkoxy, is intended to include C₁, C₂, C₃, C₄, C₅, and C₆ alkoxy groups. C₁₋₈ alkoxy, is intended to include C₁, C₂, C₃, C₄, C₅, C₆, C₇, and C₈ alkoxy groups. Examples of alkoxy include, but are not limited to, methoxy, ethoxy, n-propoxy, i-propoxy, n-butoxy, s-butoxy, t-butoxy, n-pentoxy, s-pentoxy, n-heptoxy, and n-octoxy.

As used herein, “alkylthio” refers to an alkyl group as defined above with the indicated number of carbon atoms attached through an sulfur bridge. C₁₋₆ alkylthio, is intended to include C₁, C₂, C₃, C₄, C₅, and C₆ alkylthio groups. C₁₋₈ alkylthio, is intended to include C₁, C₂, C₃, C₄, C₅, C₆, C₇, and C₈ alkylthio groups.

As used herein, “carbocycle” or “carbocyclic ring” is intended to mean, unless otherwise specified, any stable 3, 4, 5, 6, 7, 8, 9, 10, 11, or 12-membered monocyclic, bicyclic or tricyclic ring, any of which may be saturated, unsaturated, or aromatic, recognizing that rings with certain numbers of members cannot be bicyclic or tricyclic, e.g., a 3-membered ring can only be a monocyclic ring. Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, and tetrahydronaphthyl. As shown above, bridged rings are also included in the definition of carbocycle (e.g., [2.2.2]bicyclooctane). A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge. Fused (e.g., naphthyl and tetrahydronaphthyl) and spiro rings are also included. Also, when the variable “B” is selected from a carbocycle or carbocyclic ring, it does not include 5-membered rings and the upper limit for the number of members in the ring is 10.

As used herein, the term “heterocycle” means, unless otherwise stated, a stable 3, 4, 5, 6, 7, 8, 9, 10, 11, or 12-membered monocyclic, bicyclic or tricyclic ring (recognizing that rings with certain numbers of members cannot be bicyclic or tricyclic, e.g., a 3-membered ring can only be a monocyclic ring), which is saturated, unsaturated, or aromatic, and consists of carbon atoms and one or more ring heteroatoms, e.g., 1 or 1-2 or 1-3 or 1-4 or 1-5 or 1-6 heteroatoms, independently selected from the group consisting of nitrogen, oxygen, and sulfur, and including any bicyclic group in which any of the above-defined heterocyclic rings is fused to a second ring (e.g., a benzene ring). The nitrogen and sulfur heteroatoms may optionally be oxidized (i.e., N→O and S(O)ₚ, where p=1 or 2). When a nitrogen atom is included in the ring it is either N or NH, depending on whether or not it is attached to a double bond in the ring (i.e., a hydrogen is present if needed to maintain the tri-valency of the nitrogen atom). The nitrogen atom may be substituted or unsubstituted (i.e., N or NR wherein R is H or another substituent, as defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. Bridged rings are also included in the definition of heterocycle. A bridged ring occurs when one or more atoms (i.e., C, O, N, or S) link two non-adjacent carbon or nitrogen atoms. Preferred bridges include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge. Spiro and fused rings are also included. Also, when the variable “B” is selected from a heterocycle, it does not include 5-membered rings and the upper limit for the number of members in the ring is 10.

As used herein, the term “aromatic heterocycle” or “heteroaryl” is intended to mean a stable 5, 6, 7, 8, 9, 10, 11, or 12-membered monocyclic or bicyclic aromatic ring (recognizing that rings with certain numbers of members cannot be a bicyclic aromatic, e.g., a 5-membered ring can only be a monocyclic aromatic ring),which consists of carbon atoms and one or more heteroatoms, e.g., 1 or 1-2 or 1-3 or 1-4 or 1-5 or 1-6 heteroatoms, independently selected from the group consisting of nitrogen, oxygen, and sulfur. In the case of bicyclic heterocyclic aromatic rings, only one of the two rings needs to be aromatic (e.g., 2,3-dihydroindole), though both may be (e.g., quinoline). The second ring can also be fused or bridged as defined above for heterocycles. The nitrogen atom may be substituted or unsubstituted (i.e., N or NR wherein R is H or another substituent, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1. Also, when the variable “B” is selected from an aromatic heterocycle or heteroaryl, it does not include 5-membered rings and the upper limit for the number of members in the ring is 10.

Examples of heterocycles include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzilmidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-qinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

As used herein, the terms used to describe various carbon-containing moieties, including, for example, “alkyl,” “alkenyl,” “alkynyl,” “phenyl,” and any variations thereof, are intended to include univalent, bivalent, or multivalent species. For example, “C₁₋₆ alkyl-R³” is intended to represent a univalent C₁₋₆ alkyl group substituted with a R³ group, and “O—C₁₋₆alkyl-R³” is intended to represent a bivalent C₁₋₆ alkyl group, i.e., an “alkylene” group, substituted with an oxygen atom and a R³ group.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluene sulfonic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., USA, p. 1445 (1990).

Since prodrugs are known to enhance numerous desirable qualities of pharmaceuticals (e.g., solubility, bioavailability, manufacturing, etc.) the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a mammalian subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Examples of prodrugs include, but are not limited to, acetate, formate, and benzoate derivatives of alcohol and amine functional groups in the compounds of the present invention.

“Stable compound” and “stable structure” are meant to indicate a compound that is sufficiently robust to survive isolation to a useful degree of purity from a reaction mixture, and formulation into an efficacious therapeutic agent. It is preferred that the presently recited compounds do not contain a N-halo, S(O)₂H, or S(O)H group.

As used herein, “treating” or “treatment” means the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

As used herein, “mammal” refers to human and non-human patients.

As used herein, the term “therapeutically effective amount” refers to an amount of a compound, or a combination of compounds, of the present invention effective when administered alone or in combination as an anti-proliferative and/or anti-infective agent. The combination of compounds is preferably a synergistic combination. Synergy, as described, for example, by Chou and Talalay, Adv. Enzyme Regul. vol. 22, pp. 27-55 (1984), occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased anti-proliferative and/or anti-infective effect, or some other beneficial effect of the combination compared with the individual components.

All percentages and ratios used herein, unless otherwise indicated, are by weight.

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps. Further, it should be understood that the order of steps or order for performing certain actions are immaterial so long as the invention remains operable. Moreover, two or more steps or actions may be conducted simultaneously.

2. Compounds of the Invention

In one aspect, the invention provides compounds having the formula: 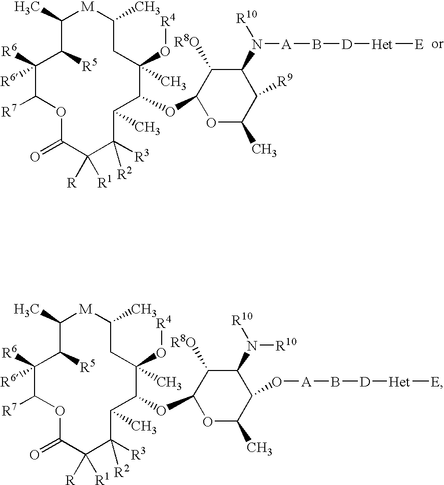
or a pharmaceutically acceptable salt, ester or prodrug thereof,

wherein:

D-Het is selected from the group consisting of:

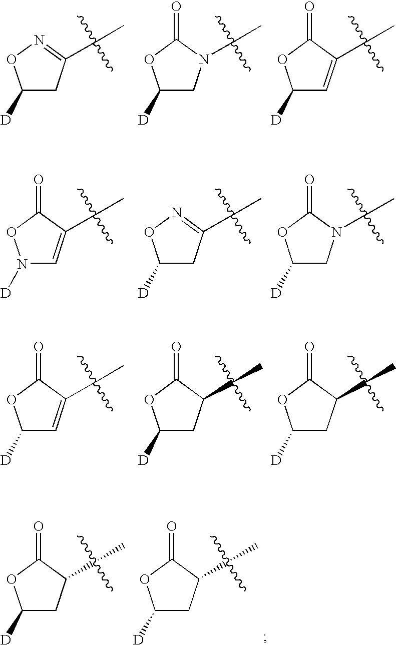

A is selected from the group consisting of:
a) C₁₋₆ acyl, b) C₁₋₆ alkyl, c) C₂₋₆ alkenyl, and d) —C(O)—C₂₋₆ alkenyl,
wherein
   i) one or more carbon atoms of any of a)-d) optionally is replaced by a moiety selected from the group consisting of O, S(O)ₚ, and NR¹¹, and
   ii) any of a)-d) optionally is substituted with one or more R¹² groups;

B is selected from the group consisting of:
a) 3-4 membered saturated or unsaturated heterocycle containing one or more heteroatoms selected from the group consisting of nitrogen, oxygen, and sulfur,
b) 6-10 membered saturated, unsaturated, or aromatic heterocycle containing one or more heteroatoms selected from the group consisting of nitrogen, oxygen, and sulfur, c) 3-4 membered saturated, unsaturated, or aromatic carbocycle, d) 6-10 membered saturated, unsaturated, or aromatic carbocycle,
   wherein any of a)-d) optionally is substituted with one or more R¹² groups;

B-D is selected from the group consisting of:
a) B—C₁₋₆ alkyl b) B—C₂₋₆ alkenyl, c) B—C₁₋₆ alkynyl, d) B—O—C₁₋₆ alkyl,
e) B—O—C₁₋₆ alkenyl, f) B—O—C₁₋₆ alkynyl, g) B—NR¹¹—C₁₋₆ alkyl,
h) B—NR¹¹—C₁₋₆ alkenyl, i) B—NR¹¹—C₁₋₆ alkynyl, j) B—S(O)ₚ—C₁₋₆ alkyl,
k) B—S(O)ₚ—C₁₋₆ alkenyl, and l) B—S(O)ₚ—C₁₋₆ alkynyl,
   wherein any of a)-l) optionally is substituted with one or more moieties
   selected from the group consisting of ═O, ═S, ═NOR¹¹, and R¹²;
E is selected from the group consisting of: 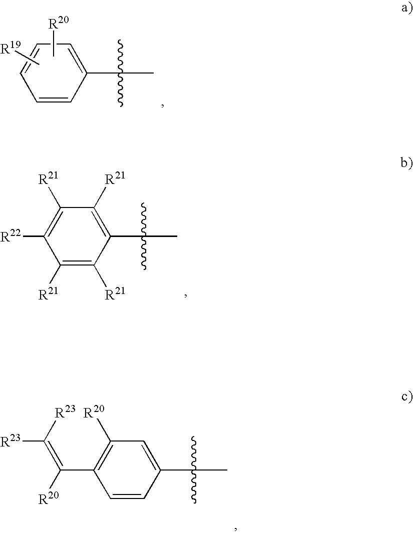
d) 5-10 membered saturated, unsaturated, or aromatic heterocycle containing one or more heteroatoms selected from the group consisting of nitrogen, oxygen, and sulfur, and optionally substituted with one or more R¹² groups,
e) C₅₋₁₀ saturated, unsaturated, or aromatic carbocycle, optionally substituted with one or more R¹² groups,
f) C₁₋₈ alkyl,
g) C₂₋₈ alkenyl,
h) C₂₋₈ alkynyl,
i) C₁₋₈ alkoxy,
j) C₁₋₈ alkylthio,
k) C₁₋₈ acyl,
l) S(O)ₚR¹¹, and
m) hydrogen,
   wherein any of f)-k) optionally is substituted with
      i) one or more R¹² groups,
      ii) 5-6 membered saturated, unsaturated, or aromatic heterocycle containing one or more heteroatoms selected from the group consisting of nitrogen, oxygen, and sulfur, and optionally substituted with one or more R¹² groups, or
      iii) C₅₋₁₀ saturated, unsaturated, or aromatic carbocycle, optionally substituted with one or more R¹² groups;

M is selected from the group consisting of:
a) —C(O)—, b) —C(═NOR¹¹)—, c) —CH(—OR¹¹)—, d) —NR¹¹—CH₂—, e) —CH₂—NR¹¹—, f) —CH(NR¹¹R¹¹)—, g) C(═NNR¹¹R¹¹)—, h) —NR¹¹C(O)—, i) —C(O)NR¹¹—, and j) —C(═NR¹¹)—;

R is selected from the group consisting of H and C₁₋₆ alkyl;

R¹ is selected from the group consisting of:
a) H, b) Cl, c) F, d) Br, e) I, f) —NR¹¹R¹¹, g) —NR¹¹C(O)R¹¹, h) —OR¹¹, i) —OC(O)R¹¹, j) —OC(O)OR¹¹, k) —OC(O)NR¹¹R¹¹, l) —O—C₁₋₆ alkyl, m) —OC(O)—C₁₋₆ alkyl, n) —OC(O)O—C₁₋₆ alkyl, o) —OC(O)NR¹¹—C₁₋₆ alkyl, p) C₁₋₆ alkyl, q) C₁₋₆ alkenyl, r) C₁₋₆ alkynyl,
   wherein any of l)-r) optionally is substituted with one or more R¹² groups;

R² is H;

R³ is selected from the group consisting of:
a) H, b) —OR¹¹, c) —O—C₁₋₆ alkyl-R¹², d) —OC(O)R¹¹, e) —OC(O)—C₁₋₆ alkyl-R¹², f) —OC(O)OR¹¹, g) —OC(O)O—C₁₋₆ alkyl-R¹² , h) —OC(O)NR¹¹R¹¹, i) —C(O)NR¹¹—C₁₋₆ alkyl-R¹², and j) 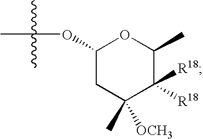
alternatively, R² and R³ taken together form a carbonyl group;
R⁴ is selected from the group consisting of:
a) H, b) R¹¹, c) —C(O)R¹¹d) —(O)OR¹¹ e) —C(O)NR¹¹R¹¹, f) —C₁₋₆ alkyl-G-R¹¹, g) -C₂₋₆ alkenyl-G-R¹¹, and h) -C₂₋₆ alkynyl-G-R¹¹;

alternatively R³ and R⁴, taken together with the atoms to which they are bonded, form:

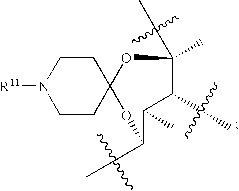

G is selected from the group consisting of:
a) —C(O)—, b) —C(O)O—, c) —C(O)NR¹¹—, d) —C(═NR¹¹)—, e) —C(═NR¹¹)O—, f) —C(═NR¹¹)NR¹¹—, g) —OC(O)—, h) —OC(O)O—, i) —OC(O)NR¹¹—, j) —NR¹¹ C(O)—, k) —NR¹¹C(O)O—, l) —NR¹¹C(O)NR¹¹—, m) —NR¹¹C(═NR¹¹)NR¹¹, and o) —S(O)ₚ—;

R⁵ is selected from the group consisting of:
a) R¹¹, b) —OR¹¹, c) —NR¹¹R¹¹, d) —O—C₁₋₆ alkyl-R¹², e) —C(O)—R¹¹, f) —C(O)—C₁₋₆ alkyl-R¹², g) —OC(O)R¹¹, h) —OC(O)C₁₋₆ alkyl-R¹², i) —OC(O)O-R¹¹, j) —OC(O)O—C₁₋₆ alkyl-R¹², k) —OC(O)NR¹¹R¹¹, l) OC(O)NR¹¹—C₁₋₆ alkyl-R¹², m) —C(O)—C₂₋₆ alkenyl-R¹², and n) —C(O)—C₂₋₆ alkynyl-R¹²;

alternatively, R⁴ and R⁵, taken together with the atoms to which they are bonded, form: 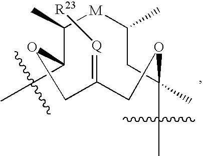
wherein
   Q is CH or N, and
   R²³ is —OR¹¹, or R¹¹;

R⁶ is selected from the group consisting of:
a) —OR¹¹, b) -C₁₋₆ alkoxy-R¹², c) —C(O)R¹¹, d) —OC(O)R¹¹, e) —OC(O)OR¹¹, f) —OC(O)NR¹¹R¹¹, and g) —NR¹¹R¹¹,

alternatively, R⁵ and R⁶ taken together with the atoms to which they are attached form a 5-membered ring by attachment to each other through a linker selected from the group consisting of:
a) —OC(R¹²)₂O—, b) —OC(O)O—, c) —OC(O)NR¹¹—, d) —NR¹¹C(O)O—, e) —OC(O)NOR¹¹—, f) —NOR¹¹—C(O)O—, g) —OC(O)NNR¹¹R¹¹—, h) —NNR¹¹R¹¹—C(O)O—, i) —OC(O)C(R¹²)₂—, j) —C(R¹²)₂C(O)O—, k) —OC(S)O—, l) —OC(S)NR¹¹R¹¹—, m) —NR¹¹C(S)O—, n) —OC(S)NOR¹¹—, o) —NOR¹¹—C(S)O—, p) —OC(S)NNR¹¹R¹¹—, q) —NNR¹¹R¹¹-C(S)O—, r) —OC(S)C(R¹²)₂—, and s) —(R¹²)₂C(S)O—;

alternatively, M, R⁵, and R⁶ taken together with the atoms to which they are attached form: 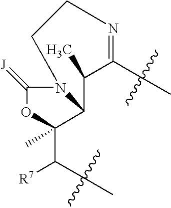
wherein J is selected from the group consisting of O and NR¹¹;

R^{6′} is selected from the group consisting of:
a) H, b) -C₁₋₄ alkyl, c) C₂₋₄ alkenyl, which can be further substituted with C₁₋₁₂ alkyl or one or more halogens, d) C₂₋₄ alkynyl, which can be further substituted with C₁₋₁₂ alkyl or one or more halogens, e) aryl or heteroaryl, which can be further substituted with C₁₋₁₂ alkyl or one or more halogens, f) —C(O)H, g) —COOH, h) —CN, i) —COOR¹¹, j) —C(O)NR¹¹R¹¹, k) —(O)R¹¹, and l) —C(O)SR¹¹, wherein b) is further substituted with one or more substituents selected from the group consisting of aa) —OR¹¹, bb) halogen, cc) —SR¹¹, dd) C₁₋₁₂ alkyl, which can be further substituted with halogen, hydroxyl, C₁₋₆ alkoxy, or amino, ee) —OR¹¹, ff) —SR¹¹, gg) —NR¹¹R¹¹, hh) —CN, ii) —NO₂, jj) —NC(O)R¹¹, kk) —COOR¹¹, ll) —N₃, mm) ═N—O-R¹¹, nn) ═NR¹¹, oo) ═N—NR¹¹R¹¹, pp) ═N—NH—C(O)R¹¹, and qq) ═N—NH—C(O)NR¹¹R¹¹;

alternatively R⁶ and R^{6′} are taken together with the atom to which they are attached to form an epoxide, a carbonyl, an olefin, or a substituted olefin, or a C₃₋₇ carbocyclic, carbonate, or carbamate, wherein the nitrogen of said carbamate can be further substituted with a C₁-C₆ alkyl;

R⁷ is selected from the group consisting of:
a) C₁₋₆ alkyl, b) C₂₋₆ alkenyl, and c) C₂₋₆ alkynyl,
   wherein any of a)-c) optionally is substituted with one or more R¹² groups;

R⁸ is selected from the group consisting of H and —C(O)R¹¹;

R⁹ is selected from the group consisting of H, OH, and —OR¹¹;

R¹⁰ is selected from the group consisting of:
a) H, b) R¹¹, c) -C₁₋₆ alkyl-G-R¹¹, d) -C₂₋₆ alkenyl-G-R¹¹, and e) -C₂₋₆ alkynyl-G-R¹¹,
   wherein any of c)-e) optionally is substituted with one or more R¹² groups;

R¹¹, at each occurrence, independently is selected from the group consisting of:
a) H, b) C₁₋₆ alkyl, c) C₂₋₆ alkenyl, d) C₂₋₆ alkynyl, e) C₆₋₁₀ saturated, unsaturated, or aromatic carbocycle, f) 3-12 membered saturated, unsaturated, or aromatic heterocycle containing one or more heteroatoms selected from the group consisting of nitrogen, oxygen, and sulfur, g) —C(O)-C₁₋₆ alkyl, h) —C(O)-C₂₋₆ alkenyl, alkenyl, i) —C(O)-C₂₋₆ alkynyl, j) —C(O)-C₆₋₁₀ saturated, unsaturated, or aromatic carbocycle, k) —C(O)3-12 membered saturated, unsaturated, or aromatic heterocycle containing one or more heteroatoms selected from the group consisting of nitrogen, oxygen, and sulfur, l) —C(O)O-C₁₋₆ alkyl, m) —C(O)—C₂₋₆ alkenyl, n) —C(O)—C₂₋₆ alkynyl, o) —C(O)O—C₆₋₁₀ saturated, unsaturated, or aromatic carbocycle, p) —C(O)O-3-12 membered saturated, unsaturated, or aromatic heterocycle containing one or more heteroatoms selected from the group consisting of nitrogen, oxygen, and sulfur, and q) —C(O)NR¹³R¹³,
   wherein any of b)-p) optionally is substituted with one or more R¹² groups,

alternatively, NR¹¹R¹¹ forms a 3-7 membered saturated, unsaturated or aromatic ring including the nitrogen atom to which the R¹¹ groups are bonded and optionally one or more moieties selected from the group consisting of O, S(O)ₚ, N, and NR¹⁵;

R¹² is selected from the group consisting of:
a) R¹⁴, b) C₁₋₈ alkyl, c) C₂₋₈ alkenyl, d) C₂₋₈ alkynyl, e) C₃₋₁₂ saturated, unsaturated, or aromatic carbocycle, f) 3-12 membered saturated, unsaturated, or aromatic heterocycle containing one or more heteroatoms selected from the group consisting of nitrogen, oxygen, and sulfur, and g) —NR¹⁵C(O)OR¹⁵,
   wherein any of b)-f) optionally is substituted with one or more R¹⁴ groups;

R¹³, at each occurrence, independently is selected from the group consisting of:
a) H, b) C₁₋₆ alkyl, c) C₂₋₆ alkenyl, d) C₂₋₆ alkynyl, e) C₃₋₁₀ saturated, unsaturated, or aromatic carbocycle, and f) 3-10 membered saturated, unsaturated, or aromatic heterocycle containing one or more heteroatoms selected from the group consisting of nitrogen, oxygen, and sulfur,
   wherein any of b)-f) optionally is substituted with one or more moieties selected from the group consisting of:
      aa) carbonyl, bb) formyl, cc) F, dd) Cl, ee) Br, ff) I, gg) CN, hh) NO₂, ii) OR¹⁵, jj) —S(O)ₚR¹⁵, kk) —C(O)R¹⁵, ll) —C(O)OR¹⁵, mm) —OC(O)R¹⁵, nn) —C(O)NR¹⁵R¹⁵, oo) —OC(O)NR¹⁵R¹⁵, pp) —C(═NR¹⁵)R¹⁵, qq) —C(R¹⁵)(R¹⁵)OR¹⁵, rr) —C(R¹⁵)₂OC(O)R¹⁵, ss) —C(R¹⁵)(OR¹⁵)(CH₂)ᵣNR¹⁵R¹⁵, tt) —NR¹⁵R¹⁵; uu) —NR¹⁵, vv)—NR¹⁵C(O)R¹⁵, ww) —NR¹⁵C(O)OR¹⁵, xx) —NR¹⁵C(O)NR¹⁵R¹⁵, yy) —NR¹⁵S(O)ᵣR¹⁵, zz) —C(OR¹⁵)(OR⁵)R¹⁵, ab) —C(R¹⁵)₂NR¹⁵R¹⁵, ac) ═NR¹⁵, ad) —C(S)NR¹⁵R¹⁵, ae) —NR¹⁵C(S)R¹⁵, af) —OC(S)NR¹⁵R¹⁵, ag) —NR¹⁵C(S)OR¹⁵, ah) —NR¹⁵C(S)NR¹⁵R¹⁵, ai) —SC(O)R¹⁵, aj) C₁₋₈ alkyl, ak) C₂₋₈ alkenyl, al) C₂₋₈ alkynyl, am) C₁₋₈ alkoxy, an) C₁₋₈ alkylthio, ao) C₁₋₈ acyl, ap) saturated, unsaturated, or aromatic C₃₋₁₀ carbocycle, and aq) saturated, unsaturated, or aromatic 3-10 membered heterocycle containing one or more heteroatoms selected from the group consisting of nitrogen, oxygen, and sulfur,

alternatively, NR¹³R¹³ forms a 3-10 membered saturated, unsaturated or aromatic ring including the nitrogen atom to which the R¹³ groups are attached and optionally one or more moieties selected from the group consisting of O, S(O)ₚ, N, and NR¹⁵;

alternatively, CR¹³R¹³ forms a carbonyl group;

R¹⁴, at each occurrence, is selected from the group consisting of:
a) H, b) ═O, c) F, d) Cl, e) Br, f) I, g) (CR¹³R¹³)ᵣCF₃, h) (CR¹³R¹³)ᵣCH, i) (CR¹³R¹³)ᵣNO₂, j) (CR¹³R¹³)ᵣNR¹³(CR¹³R¹³)ₜR¹⁶, k) (CR¹³R¹³)ᵣOR¹⁶, l) (CR¹³R¹³)ᵣS(O)ₚ(CR¹³R¹³)ₜR¹⁶, m) (CR¹³R¹³)ᵣC(O)(CR¹³R₁₃)ₜR¹⁶, n) (CR¹³R¹³)ᵣOC(O)(CR¹³R¹³)ₜR¹⁶, o) (CR¹³R¹³)ᵣSC(O)(CR¹³R¹³)ₜR¹⁶, p) (CR¹³R¹³)ᵣC(O)O(CR¹³R¹³)ₜR¹⁶, q) (CR¹³R¹³)ᵣNR¹³C(O)(CR¹³R¹³)ₜR¹⁶, r) (CR¹³R¹³)ᵣC(O)NR¹³(CR¹³R¹³)ₜR¹⁶, s) (CR¹³R¹³)ᵣC(═NR¹³)(CR¹³R¹³)ₜR¹⁶, t) (CR¹³R¹³)ᵣC(═NNR¹³R¹³)(CR¹³R¹³)ₜR¹⁶, u) (CR¹³R¹³)ᵣC(═NNR¹³C(O)R¹³)(CR¹³R¹³)ₜR¹⁶, v) (CR¹³R¹³)ᵣC(═NOR¹⁶)(CR¹³R¹³)ₜR¹⁶, w) (CR¹³R¹³)ᵣNR¹³C(O)O(CR¹³R¹³)ₜR¹⁶, x) (CR¹³R¹³)ᵣOC(O)NR¹³(CR¹³R¹³)ₜR¹⁶, y) (CR¹³R¹³)ᵣNR¹³C(O)NR¹³(CR¹³R¹³)ₜR¹⁶, z) (CR¹³R¹³)ᵣNR¹³S(O)ₚ(CR¹³R¹³)ₜR¹⁶, aa) (CR¹³R¹³)ᵣS(O)ₚNR¹³(CR¹³R¹³)ₜR¹⁶, bb) (CR¹³R¹³)ᵣNR¹³S(O)ₚNR¹³(CR¹³R¹³)ₜR¹⁶, cc) (CR¹³R¹³)ᵣNR¹³R¹³, dd) C₁₋₆ alkyl, ee) C₂₋₆ alkenyl, ff) C₂₋₆ alkynyl, gg) (CR¹³R¹³)ᵣ—C₃₋₁₀ saturated, unsaturated, or aromatic carbocycle, and hh) (CR¹³R¹³)ᵣ-3-10 membered saturated, unsaturated, or aromatic heterocycle containing one or more heteroatoms selected from the group consisting of nitrogen, oxygen, and sulfur,
   wherein any of dd)-hh) optionally is substituted with one or more R¹⁶ groups;

alternatively, two R¹⁴ groups may form —O(CH₂)ᵤO—;

R¹⁵ is selected from the group consisting of:
a) H, b) C₁₋₆ alkyl, c) C₂₋₆ alkenyl, d) C₂₋₆ alkynyl, e) C₃₋₁₀ saturated, unsaturated, or aromatic carbocycle, f) 3-10 membered saturated, unsaturated, or aromatic heterocycle containing one or more heteroatoms selected from the group consisting of nitrogen, oxygen, and sulfur, g) —C(O—C₁₋₆ alkyl, h) —C(O)—C₁₋₆ alkenyl, g) —(O)—C₁₋₆ alkynyl, i) —(O)—C3-10 saturated, unsaturated, or aromatic carbocycle, and j) —(O)3-10 membered saturated, unsaturated, or aromatic heterocycle containing one or more heteroatoms selected from the group consisting of nitrogen, oxygen, and sulfur,
   wherein any of b)-j) optionally is substituted with one or more moieties selected from the group consisting of: aa) H, bb) F, cc) Cl, dd) Br, ee) I, ff) CN, gg) NO₂, hh) OH, ii) NH₂, jj) NH(C₁₋₆ alkyl), kk) N(C₁₋₆ alkyl)₂, ll) C₁₋₆ alkoxy, mm) aryl, nn) substituted aryl, oo) heteroaryl, pp) substituted heteroaryl, and qq) C₁₋₆ alkyl, optionally substituted with one or more moieties selected from the group consisting of aryl, substituted aryl, heteroaryl, substituted heteroaryl, F, Cl, Br, I, CN, NO₂, and OH;

R¹⁶, at each occurrence, independently is selected from the group consisting of:
a) R¹⁷, b) C₁₋₆ alkyl, c) C₂₋₆ alkenyl, d) C₂₋₆ alkynyl, e) C₃₋₁₀ saturated, unsaturated, or aromatic carbocycle, and f) 3-10 membered saturated, unsaturated, or aromatic heterocycle containing one or more heteroatoms selected from the group consisting of nitrogen, oxygen, and sulfur,
   wherein any of b)-f) optionally is substituted with one or more R¹⁷ groups;

R¹⁷, at each occurrence, independently is selected from the group consisting of:
a) H, b) ═O, c) F, d) Cl, e) Br, f) I, g) (CR¹³R¹³)ᵣCF₃, h) (CR¹³R¹³)ᵣCN, i) (CR¹³R¹³)ᵣNO₂, j) (CR¹³R¹³)ᵣNR¹³R¹³, k) (CR¹³R¹³)ᵣOR¹³, l) (CR¹³R¹³)ᵣS(O)ₚR¹³, m) (CR¹³R¹³)ᵣC(O)R¹³, n) (CR¹³R¹³)ᵣC(O)OR¹³, o) (CR¹³R¹³)ᵣOC(O)R¹³, p) (CR¹³R¹³)ᵣNR¹³C(O)R¹³, q) (CR¹³R¹³)ᵣC(O)NR¹³R¹³, r) (CR¹³R¹³)ᵣC(═NR¹³)R¹³, s) (CR¹³R¹³)ᵣNR¹³C(O)NR¹³R¹³, t) (CR¹³R¹³)ᵣNR¹³S(O)ₚR¹³, u) (CR¹³R¹³)ᵣS(O)ₚNR¹³R¹³, v) (CR¹³R¹³)ᵣNR¹³S(O)ₚNR¹³R¹³, w) C₁₋₆ alkyl, x) C₂₋₆ alkenyl, y) C₂₋₆ alkynyl, z) (CR¹³R¹³)ᵣ—C₃₋₁₀ saturated, unsaturated, or aromatic carbocycle, and aa) (CR¹³R¹³)ᵣ-3-10 membered saturated, unsaturated, or aromatic heterocycle containing one or more heteroatoms selected from the group consisting of nitrogen, oxygen, and sulfur,
   wherein any of w)-aa) optionally is substituted with one or more moieties selected from the group consisting of R¹³, F, Cl, Br, I, CN, NO₂, —OR¹³, —NH₂, —NH(C₁₋₆ alkyl), —N(C₁₋₆ alkyl)₂, C₁₋₆ alkoxy, C₁₋₆ alkylthio, and C₁₋₆ acyl;

R¹⁸, at each occurrence, independently is selected from the group consisting of.
a) H, b) —OR¹⁵, c) —O—C₁₋₆ alkyl-OC(O)R¹⁵, d) —O—C₁₋₆ alkyl-OC(O)OR¹⁵, e) —O—C₁₋₆ alkyl-OC(O)NR¹⁵R¹⁵, f) —O—C₁₋₆ alkyl-C(O)NR¹⁵R¹⁵, g) —O—C₁₋₆ alkyl-NR¹⁵C(O)R¹⁵, h) —O—C₁₋₆ alkyl-NR¹⁵C(O)OR¹⁵, i) —O—C₁₋₆ alkyl-NR¹⁵C(O)NR¹⁵R¹⁵, j) —O—C₁₋₆ alkyl-NR¹⁵C(═NH)NR¹⁵R¹⁵, k) —O—C₁₋₆ alkyl-S(O)ₚR¹⁵, l) —O—C₂₋₆ alkenyl-OC(O)R¹⁵, m) —O—C₂₋₆ alkenyl-OC(O)OR¹⁵, n) —OC₂₋₆ alkenyl-OC(O)NR¹⁵R¹⁵, o) —O—C₂₋₆ alkenyl-C(O)NR¹⁵R¹⁵, p) —O—C₂₋₆ alkenyl-NR¹⁵C(O)R¹⁵, q) —O—C₂₋₆ alkenyl-NR¹⁵C(O)OR¹⁵, r) —O—C₂₋₆ alkenyl-NR¹⁵C(O)NR¹⁵R¹⁵, s) —O—C₂₋₆ alkenyl-NR¹⁵C(═NH)NR¹⁵R¹⁵, t) —O—C₂₋₆ alkenyl-S(O)ₚR¹⁵, u) —O—C₂₋₆ alkynyl-OC(O)R¹⁵, v) —O—C₂₋₆ alkynyl-OC(O)OR¹⁵, w) —O—C₂₋₆ alkynyl-OC(O)NR¹⁵R¹⁵, x) —O—C₂₋₆ alkynyl-C(O)NR¹⁵R¹⁵, y) —O—C₂₋₆ alkynyl-NR¹⁵C(O)R¹⁵, z) —O—C₂₋₆ alkynyl-NR¹⁵C(O)OR¹⁵, aa) —O—C₂₋₆ alkynyl-NR¹⁵C(O)NR¹⁵R¹⁵, bb) —O—C₂₋₆alkynyl-NR¹⁵C(═NH)NR¹⁵R¹⁵, cc) —O—C₂₋₆ alkynyl-S(O)ₚR¹⁵; and dd) —NR¹⁵R¹⁵;

alternatively, two R¹⁸ groups taken together form ═O, ═NOR¹⁵, or ═NNR¹⁵R¹⁵;

R¹⁹ is R¹²

R²⁰ is selected from the group consisting of:
a) R¹³, b) F, c) Cl, d) Br, e) I, f) CN, g) NO₂, and h) —OR¹¹;

alternatively, R¹⁹ and R²⁰ taken together are —(CH₂)ᵤO;

R²¹, at each occurrence, independently is selected from the group consisting of:
a) H, b) F, c) Cl, d) Br, e) I, f) CN, g) —OR¹¹, h) —NO₂, i) —NR¹¹R¹¹, j) C₁₋₆ alkyl,
k) C₁₋₆ acyl, and l) C₁₋₄ alkoxy;

R²² is selected from the group consisting of:
a) C₁₋₆ alkyl, b) C₂₋₆ alkenyl, c) C₂₋₆ alkynyl, d) C₁₋₆ acyl, e) C₁₋₄ alkoxy,
f) C₁₋₆ alkylthio, g) saturated, unsaturated, or aromatic C₅₋₁₀ carbocycle,
h) saturated, unsaturated, or aromatic 5-10 membered heterocycle containing one or more heteroatoms selected from the group consisting of nitrogen, oxygen, and sulfur, i) —O—C₁₋₆ alkyl-saturated, unsaturated, or aromatic 5-10 membered heterocycle containing one or more heteroatoms selected from the group consisting of nitrogen, oxygen, and sulfur, j) —NR¹¹-C₁₋₆ alkyl-saturated, unsaturated, or aromatic 5-10 membered heterocycle containing one or more heteroatoms selected from the group consisting of nitrogen, oxygen, and sulfur k) saturated, unsaturated, or aromatic 10-membered bicyclic ring system optionally containing one or more heteroatoms selected from the group consisting of nitrogen, oxygen, and sulfur, l) saturated, unsaturated, or aromatic 13-membered tricyclic ring system optionally containing one or more heteroatoms selected from the group consisting of nitrogen, oxygen, and sulfur, m) —OR¹¹, n) —NR¹¹R¹¹, o) —S(O)ₚR¹¹, and p) -R²¹,
   wherein any of a)-l) optionally is substituted with one or more R¹² groups;

alternatively, R²² and one R²¹ group, taken together with the atoms to which they are bonded, form a 5-7 membered saturated or unsaturated carbocycle, optionally substituted with one or more R¹² groups; or a 5-7 membered saturated or unsaturated heterocycle containing one or more atoms selected from the group consisting of nitrogen, oxygen, and sulfur, and optionally substituted with one or more R¹² groups;

R²³ at each occurrence, independently is selected from the group consisting of:
a) hydrogen, b) an electron-withdrawing group, c) aryl, d) substituted aryl, e) heteroaryl, f) substituted heteroaryl, and g) C₁₋₆ alkyl, optionally substituted with one or more R¹² groups;

alternatively, any R²³ and any R²⁰, taken together with the atoms to which they are bonded, form a 5-7 membered saturated or unsaturated carbocycle, optionally substituted with one or more R¹² groups; or a 5-7 membered saturated or unsaturated heterocycle containing one or more atoms selected from the group consisting of nitrogen, oxygen, and sulfur, and optionally substituted with one or more R¹² groups;

p, at each occurrence, is selected from the group consisting of 0, 1, and 2;

r, at each occurrence, is selected from the group consisting of 0, 1, and 2;

t, at each occurrence, is selected from the group consisting of 0, 1, and 2; and

u, at each occurrence, is selected from the group consisting of 1, 2, 3, and 4.

Embodiments of the foregoing compounds include those compounds having the formula: 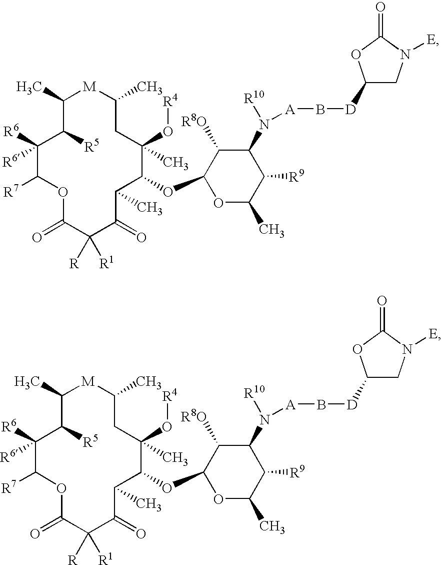 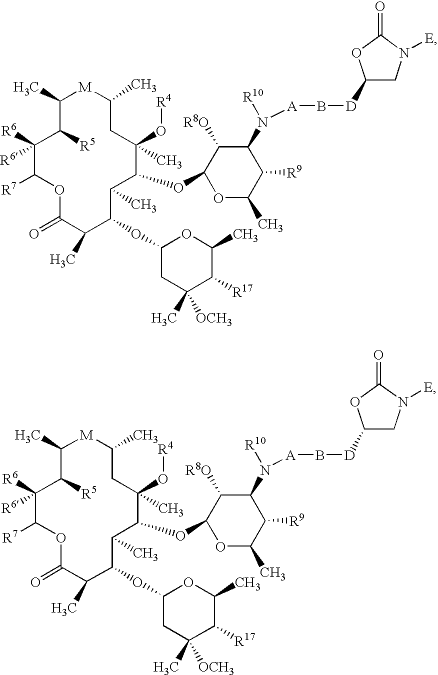 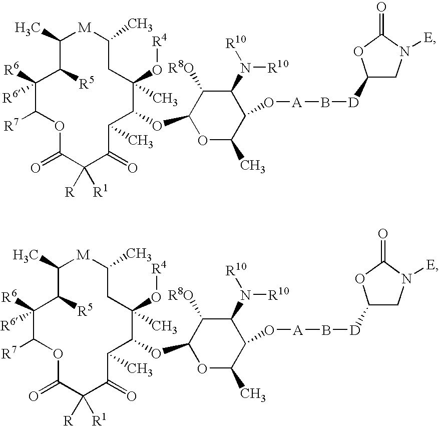 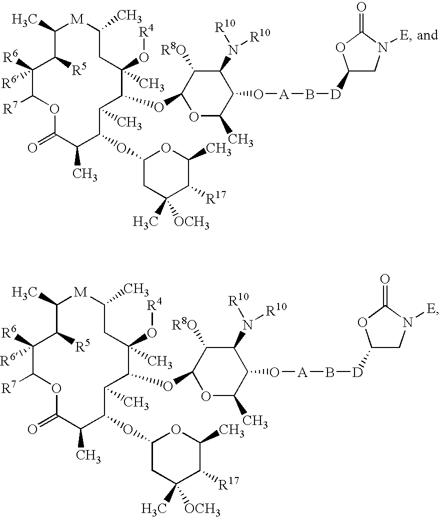
or a pharmaceutically acceptable salt, ester, or prodrug thereof, wherein A, B, D, E, M, R, R¹, R⁴, R⁵, R⁶, R^{6′}, R⁷, R⁸, R⁹, and R¹⁰ are as defined hereinabove.

Other embodiments of the foregoing compounds include those compounds having the formula: 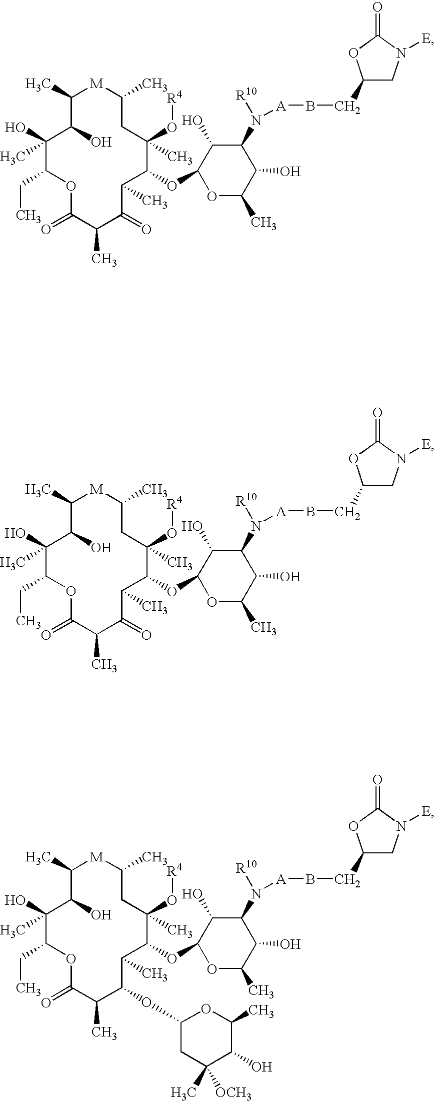 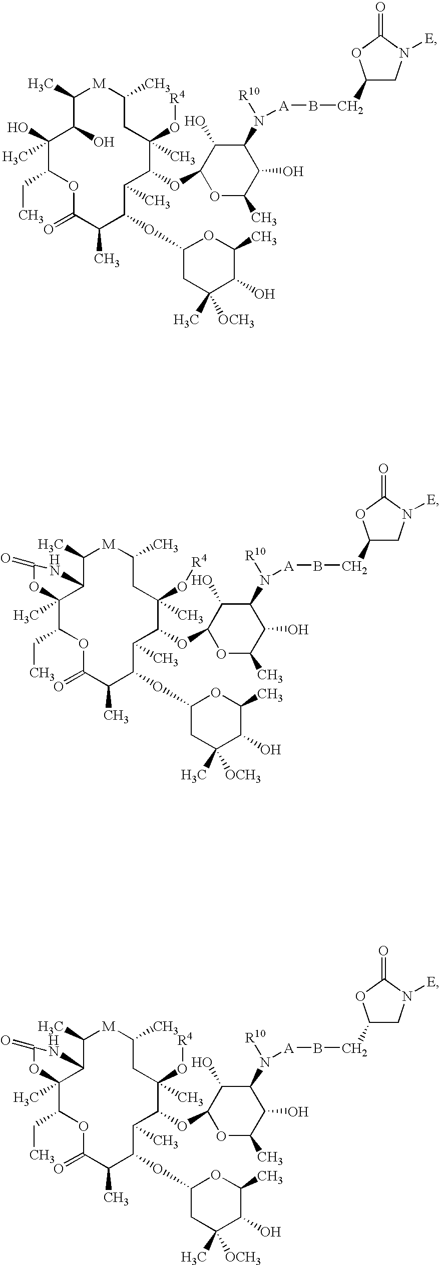 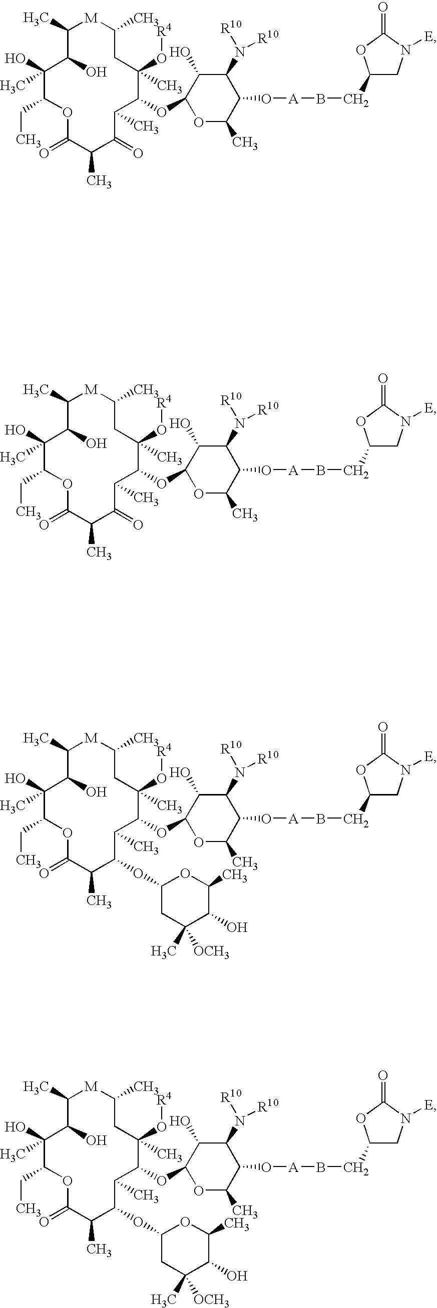 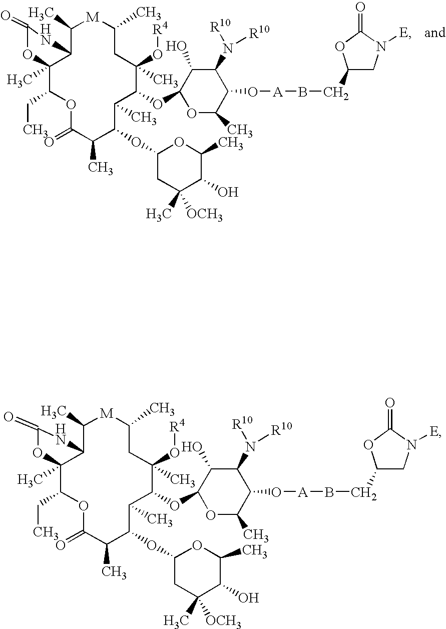
or a pharmaceutically acceptable salt, ester, or prodrug thereof, wherein A, B, E, M, R⁴, and R¹⁰ are as defined hereinabove.

Still other embodiments of the foregoing compounds include those compounds having the formula selected from the group consisting of: 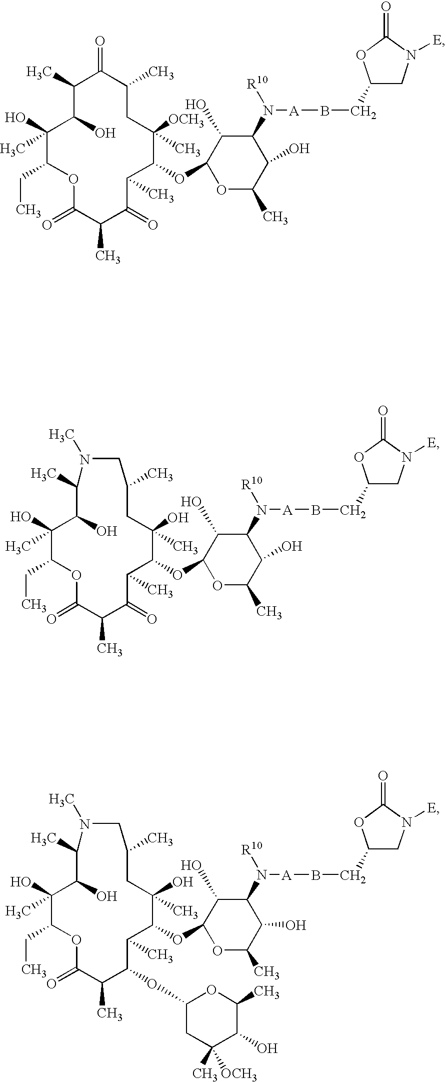 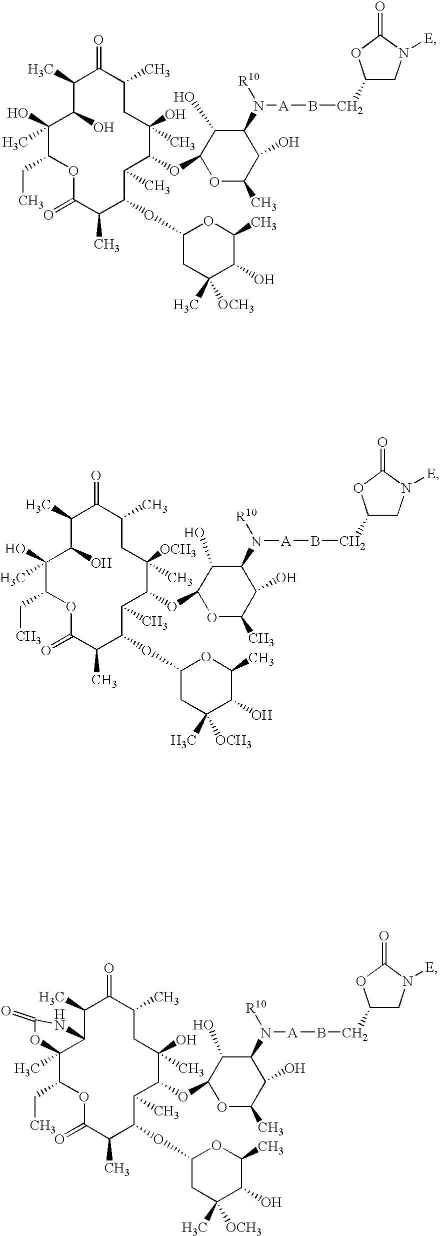 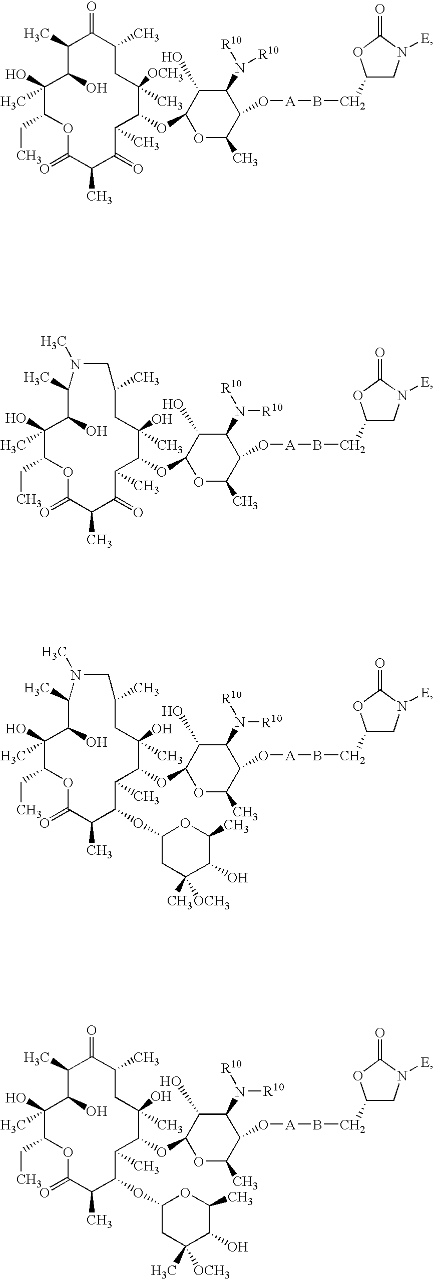 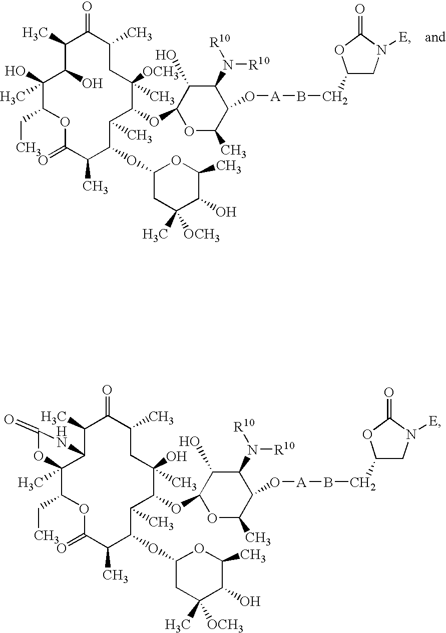
or a pharmaceutically acceptable salt, ester, or prodrug thereof, wherein A, B, E, and R¹⁰ are as defined in hereinabove.

In some embodiments of the invention, B is selected from the group consisting of a 3-4-membered saturated or unsaturated heterocycle containing one or more heteroatoms selected from the group consisting of nitrogen, oxygen, and sulfur, a 6-7-membered saturated, unsaturated, or aromatic heterocycle containing one or more heteroatoms selected from the group consisting of nitrogen, oxygen, and sulfur, and a 3-4 or 6-7-membered saturated, unsaturated, or aromatic carbocycle, wherein the heterocycle or carbocycle may be optionally substituted with one or more R¹² groups as defined hereinabove.

In other embodiments, B is selected from the group consisting of

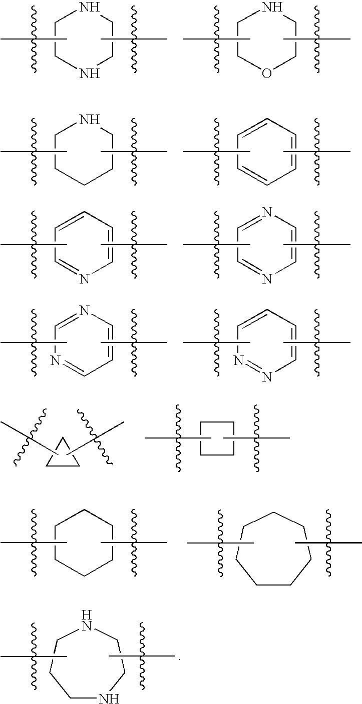

In certain embodiments of the invention, A-B-D has the formula:

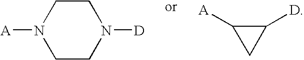

Other embodiments of the invention include a compound having the formula: 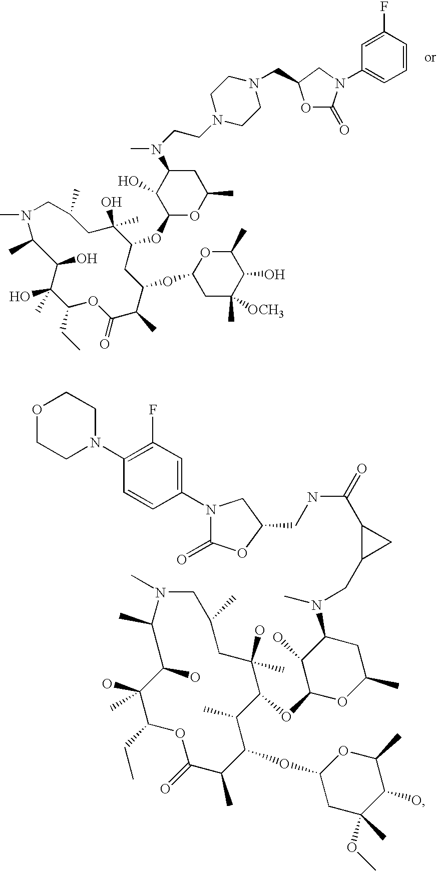
or a pharmaceutically acceptable salt, ester, or prodrug thereof.

In another aspect, the invention provides a pharmaceutical composition comprising a therapeutically effective amount of one or more of the foregoing compounds and a pharmaceutically acceptable carrier. In yet another aspect, the invention provides a method for treating a microbial infection, a fungal infection, a parasitic disease, a proliferative disease, a viral infection, an inflammatory disease, or a gastrointestinal motility disorder in a mammal by administering effective amounts of the compounds of the invention or pharmaceutical compositions of the invention. In embodiments of this aspect, the compounds are administered orally, parentally, or topically. In still another aspect, the invention provides methods for synthesizing any one of the foregoing compounds. In another aspect, the invention provides a medical device, for example, a medical stent, which contains or is coated with one or more of the foregoing compounds.

The invention further provides a family of hybrid antibiotics comprising at least a portion of a heterocyclic side-chain linked via a cyclic linker to a macrolide. Exemplary macrolides, linkers, and heterocyclic side-chains useful in the synthesis of the antibiotics include, but are not limited to, the chemical moieties shown below.

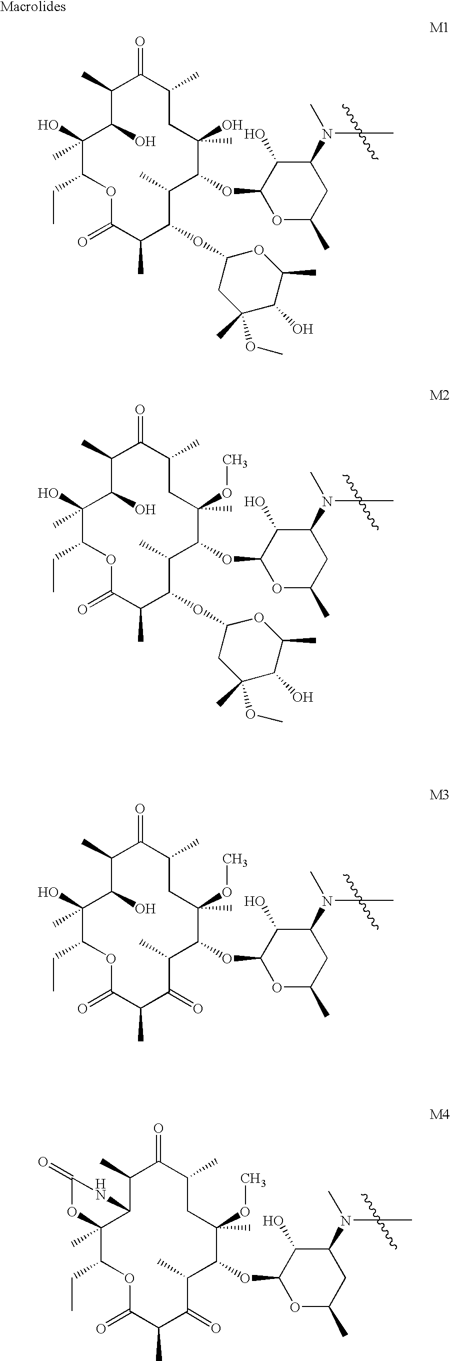

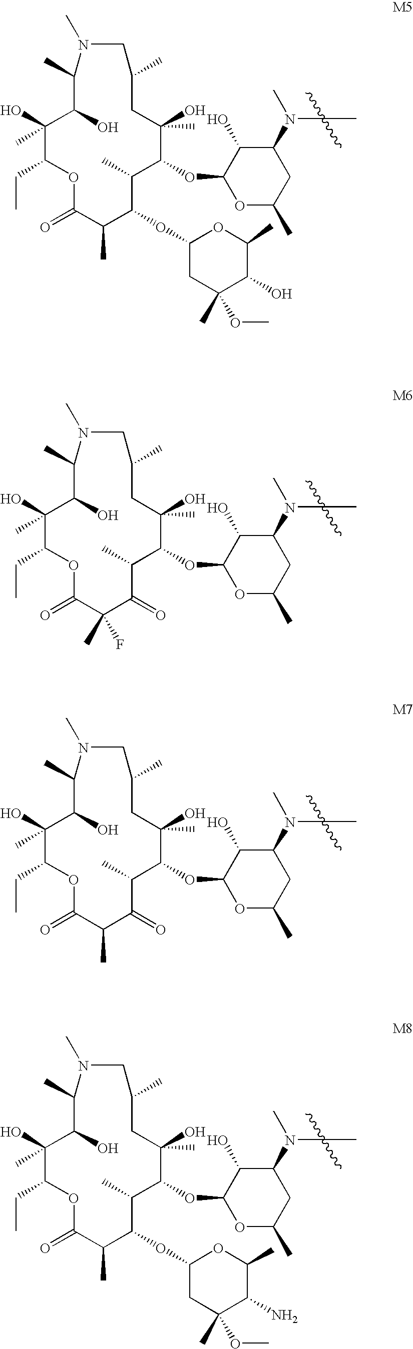

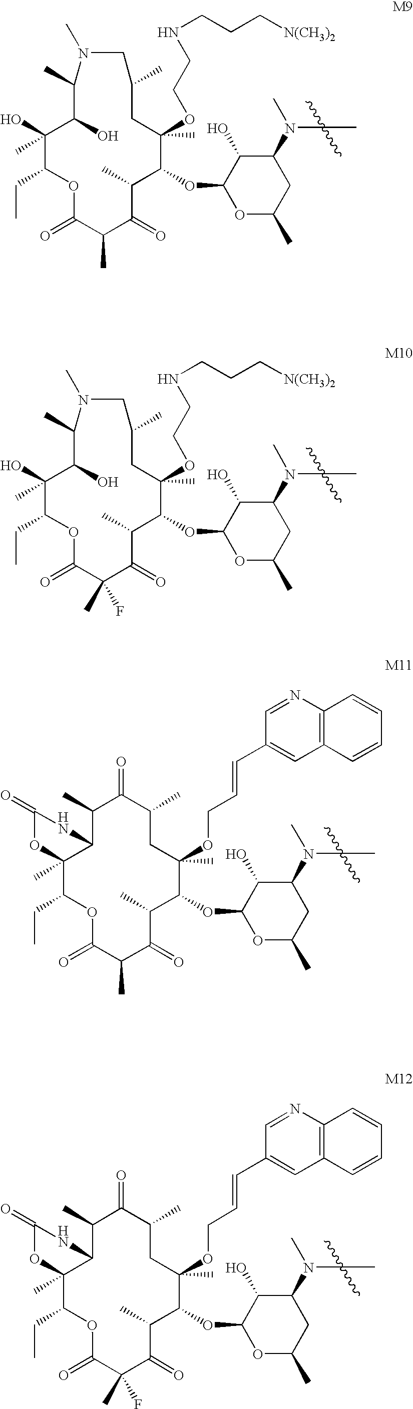

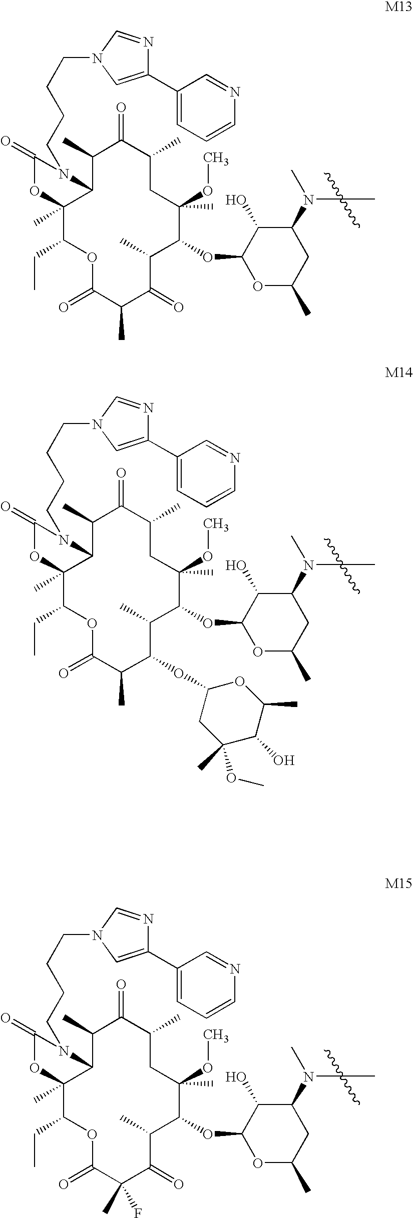

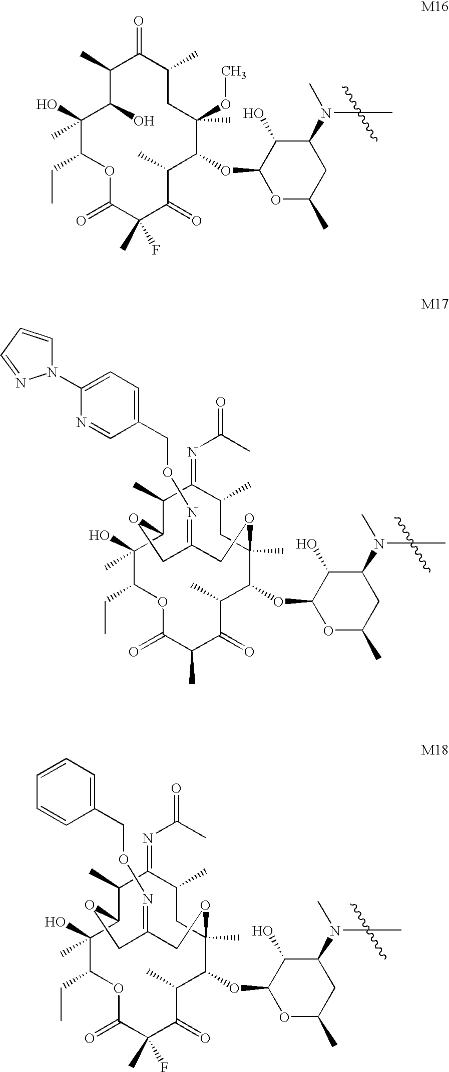

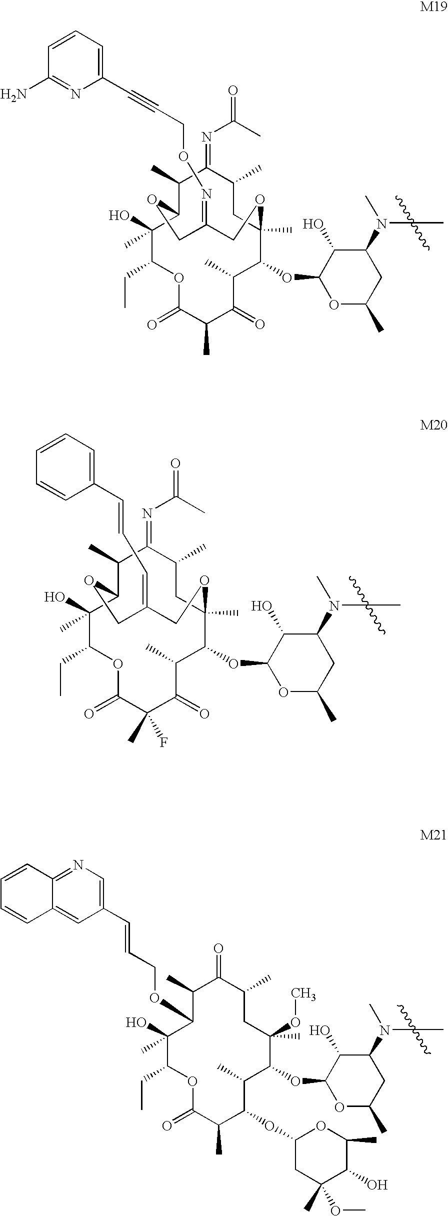

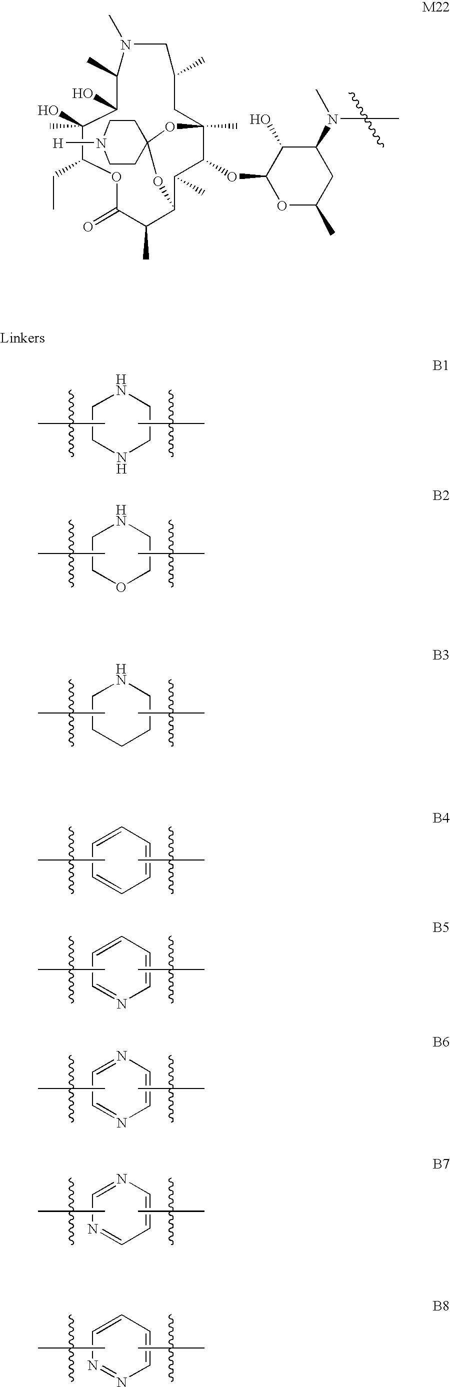

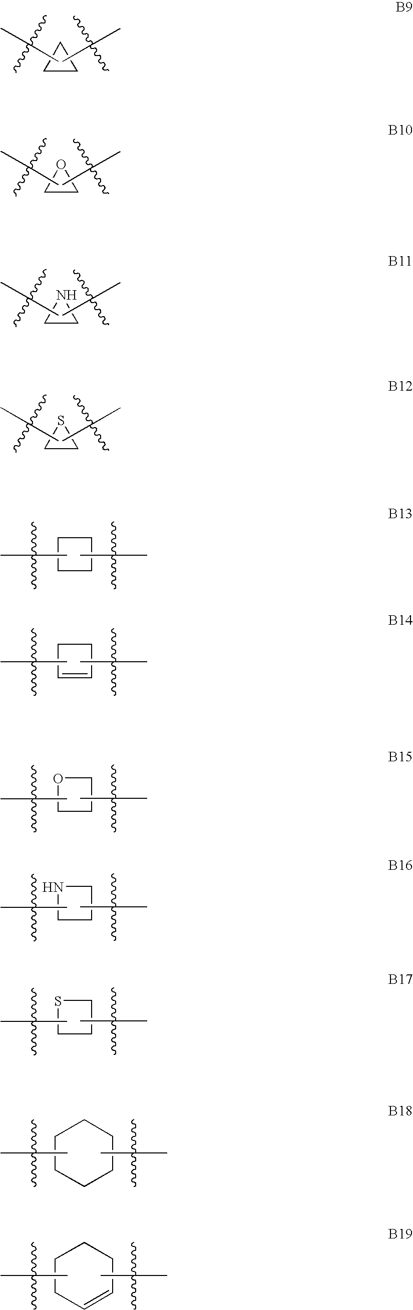

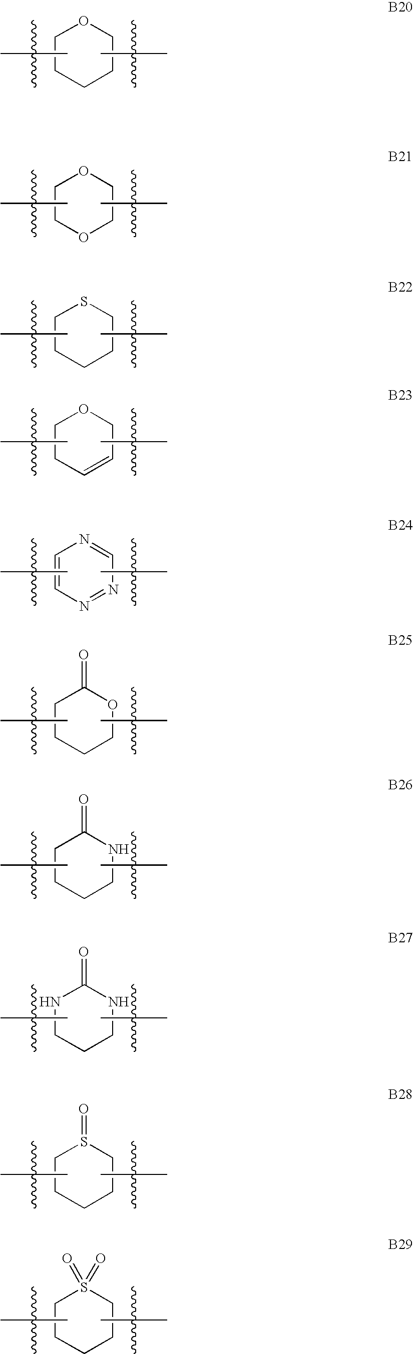

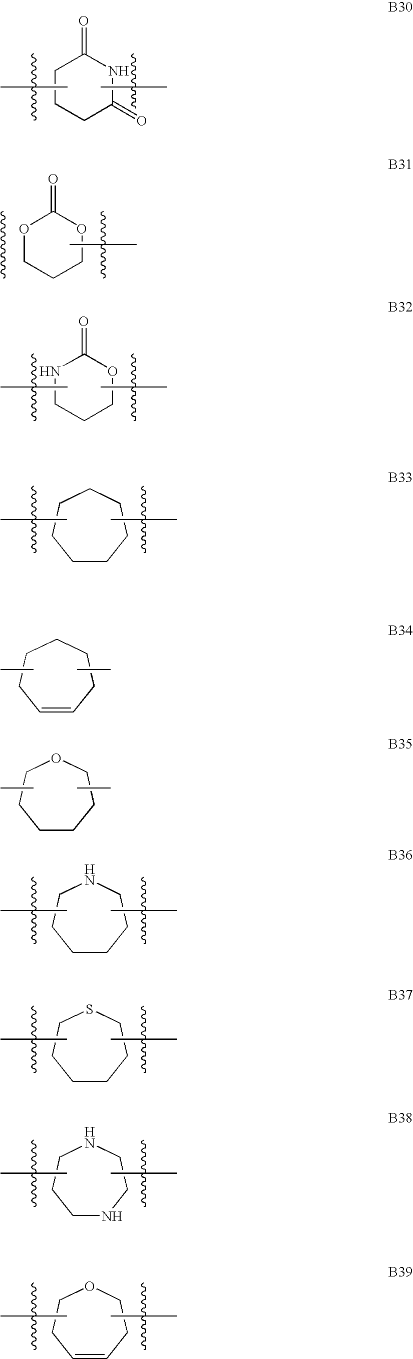

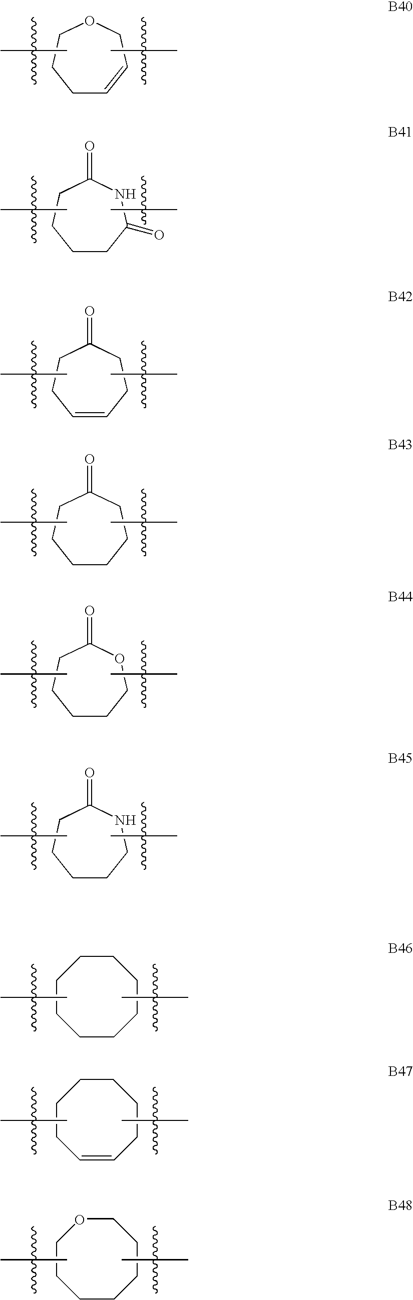

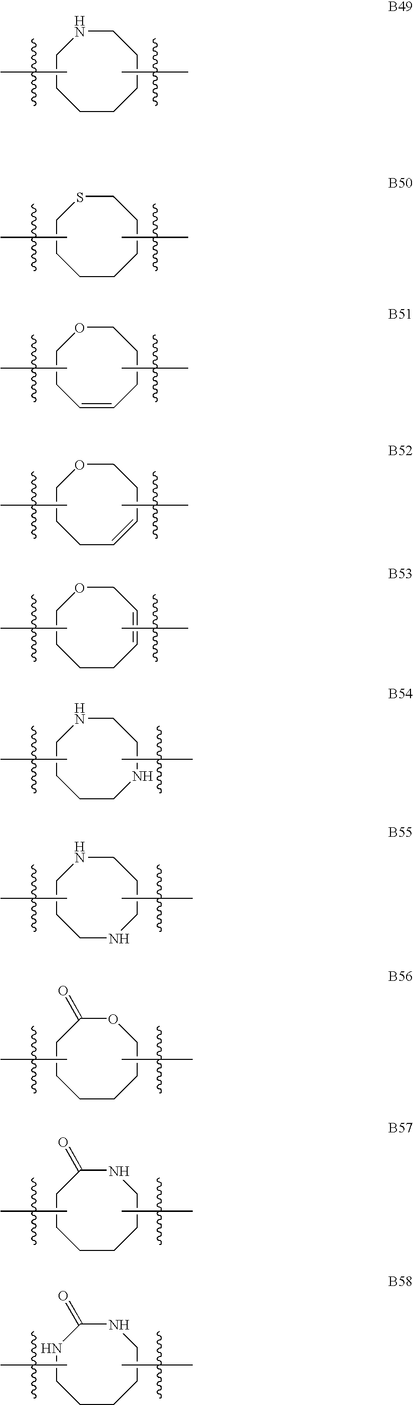

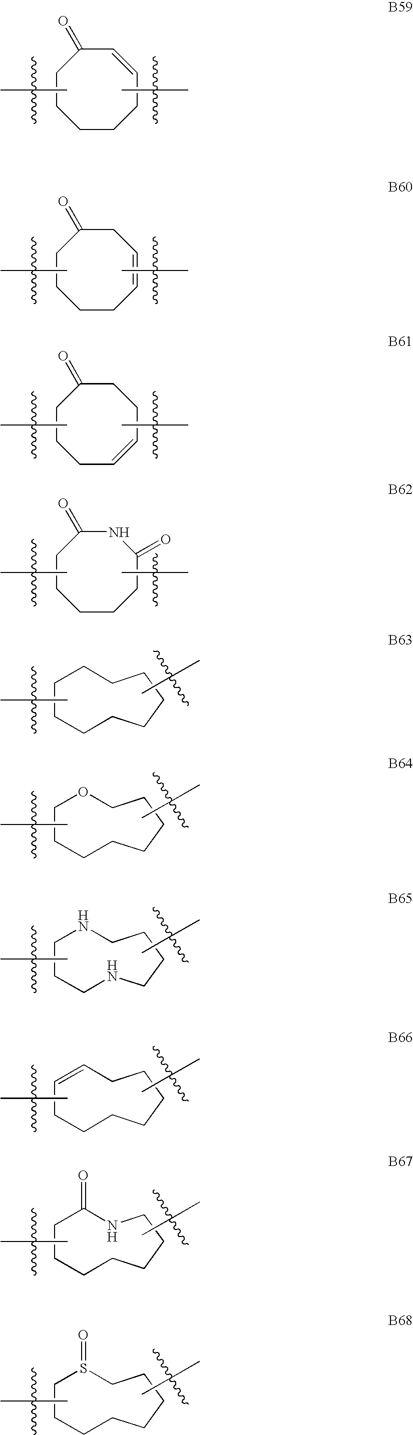

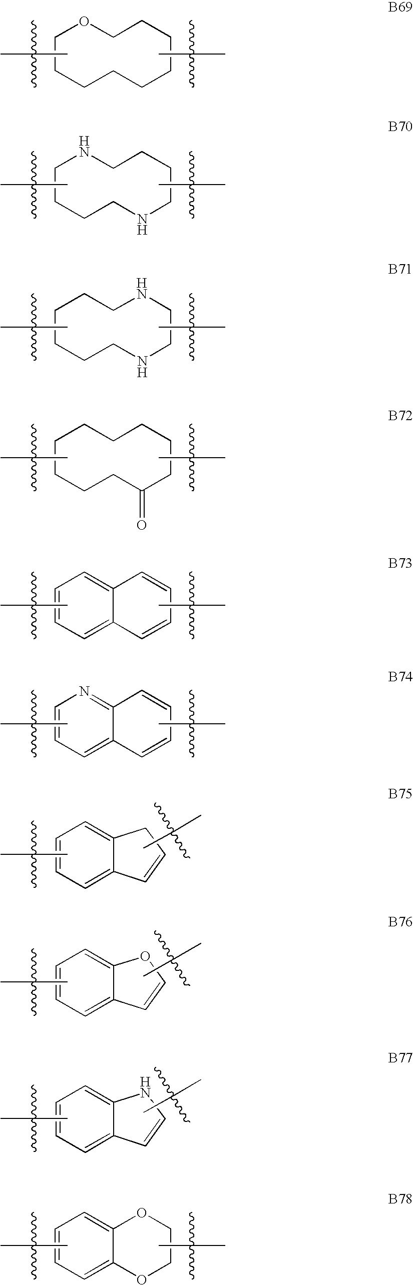

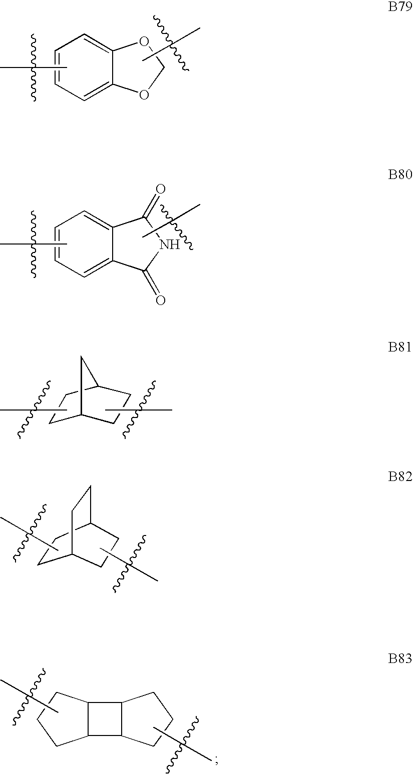

For the above linker groups, it should be understood that the macrolides and heterocyclic side-chains can individually be bonded to any atom within the ring, provided the valency of the ring atom is not exceeded. For example, with respect to linker B1, the macrolide can be bonded to a carbon atom or a nitrogen atom in the ring. If the macrolide is bonded to a carbon atom, the heterocyclic side-chain can be bonded to any carbon or nitrogen atom in the ring, including the carbon atom to which the macrolide is bonded. If the macrolide is bonded to a nitrogen atom, the heterocyclic side-chain can be bonded to any carbon atom or the nitrogen atom to which the macrolide is not bonded (i.e., the macrolide and the side-chain cannot be bonded to the same nitrogen atom).

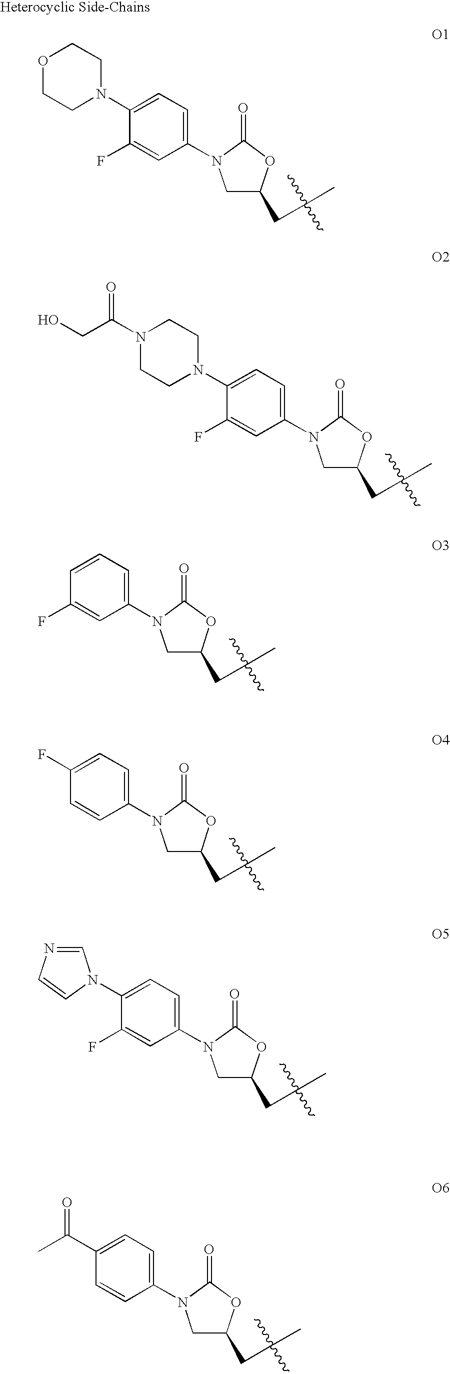

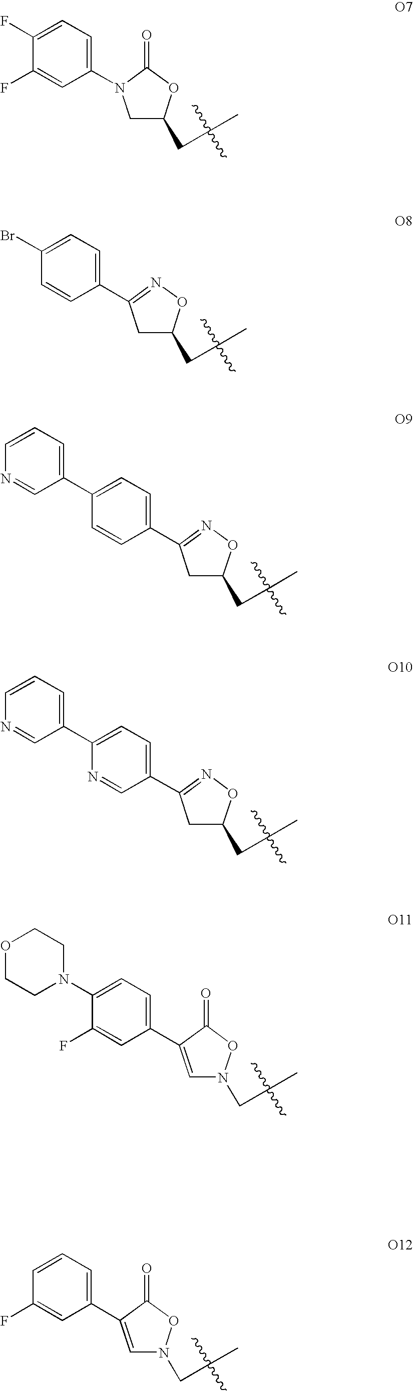

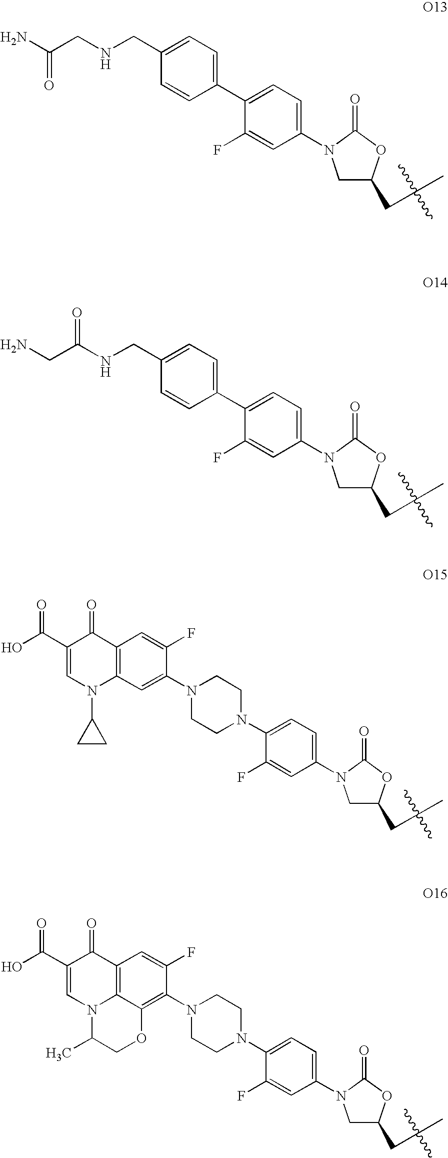

An exemplary scheme showing the linkage of a macrolide to a heterocyclic side-chain via a linker is set below, where m can be 1, 2, 3, or 4:

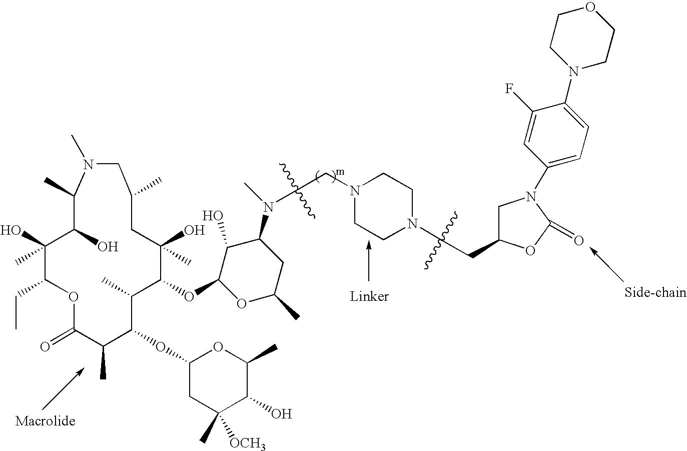

The above exemplary scheme can be condensed into the following chemical representation
M—(CH₂)ₘ—B—O
wherein M is a macrolide selected from the group consisting of M1 through M22, as shown above, B is a linker selected from the group consisting of B1 through B83 as shown above, O is a heterocyclic side chain selected from the group consisting of O1 through O16 as shown above, and m is an integer from 1-4.

The various macrolides can be linked via the linkers to the various heterocyclic side-chains using conventional chemistries known in the art, such as those discussed herein. By using the various combinations of chemical moieties provided, the skilled artisan may synthesize one or more of the exemplary compounds of the present invention. A nonlimiting example of a compound that one can make based on these exemplary moieties would be wherein the macrolide moiety is selected from M1, the linker moiety is selected from B1 with the variable length chain portion being selected from m=1, and the heterocyclic side chain moiety is selected from O1. One skilled in the art would recognize that these illustrated moieties alone can be combined to describe 116,864 unique compounds, i.e. [22 macrolides]×[83 linkers]×[4 chain lengths for each linker]×[16 heterocyclic side chains]=116,864.

3. Synthesis of the Compounds of the Invention

In another aspect, the invention provides methods for making the compounds of the invention. Schemes 1-4 below depict some exemplary chemistries available for synthesizing compounds of the invention. It will be appreciated, however, that the desired compounds may be synthesized using other alternative chemistries known in the art.

The dimethyl amino group of the desosamine sugar of macrolide antibiotics can be monodemethylated to produce the corresponding secondary amine (U.S. Pat. No. 3,725,385, Flynn et al. (1954), J. Am. Chem. Soc., 76: 3121; Ku et al. (1997), Bioorg. Med. Chem. Lett., 7: 1203, Stenmark et al. (2000), J. Org. Chem., 65: 3875). For example, desosamine derivative 1 is available from the degradation of erythromycin. Similar chemistries can be employed to produce amine 2 from azithromycin.

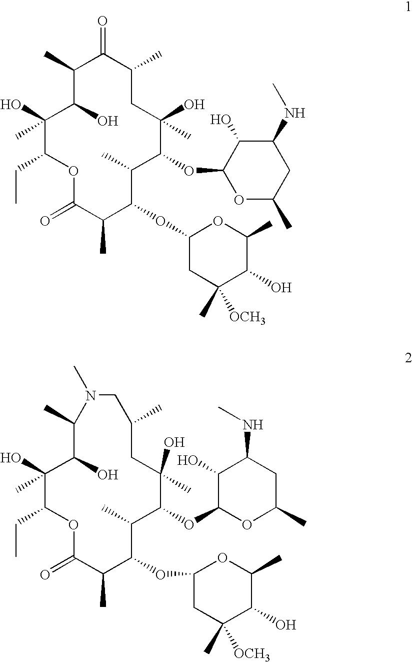

Monodemethylated macrolide antibiotics, such as amines 1 and 2, for example, can be alkylated with appropriate electrophiles using chemical reactions known to those skilled in the art to produce compounds of the present invention that include linker and heterocyclic side chain groups such as those illustrated above and claimed herein.

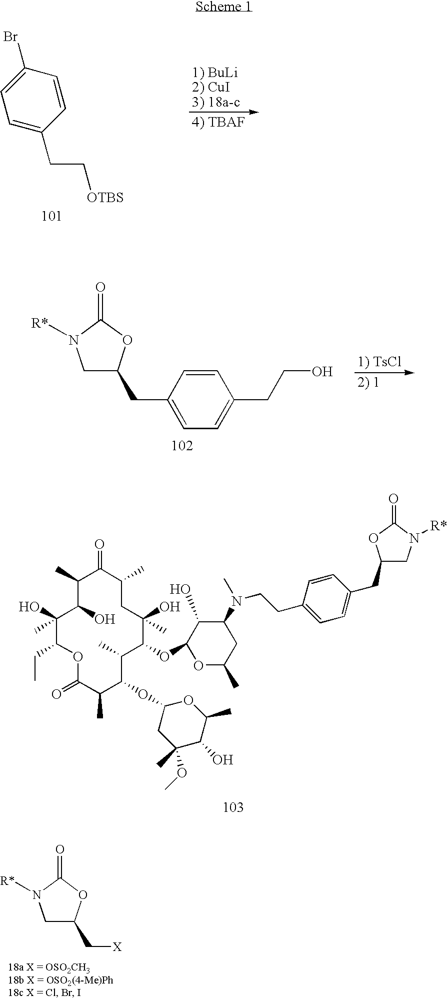

Scheme 1 illustrates the synthesis of oxazolidinones substituted at C-5 with phenyl derivatives. Aryl bromides of type 101 can be treated with alkyl lithium reagents to form aryl lithium species, and subsequent treatment with copper (I) salts leads to the formation of aryl cuprates. Addition of such cuprates to oxazolidinone derivatives 18a-c yields derivatives of type 102 after deprotection of the silyl protecting group. The primary alcohol in 102 can be converted to an alkyl sulfonate derivative or halide and subsequently displaced by macrolide derivative 1 to afford target compounds of type 103.

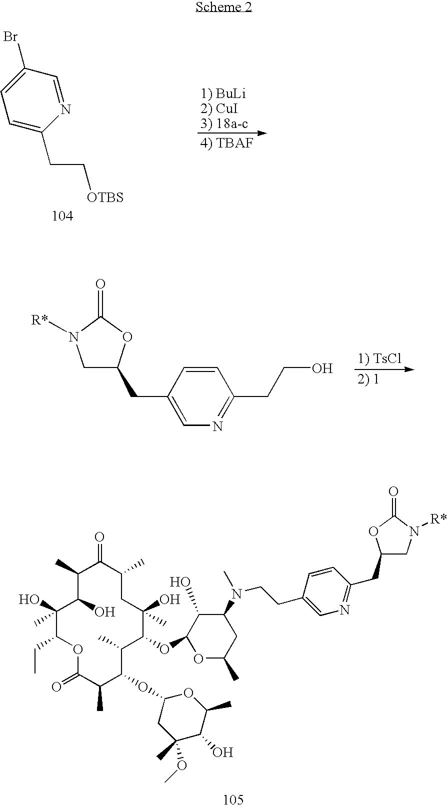

Scheme 2 illustrates how bromopyridines of type 104 can be treated in a manner similar to Scheme 1 to yield C-5 pyridine derivatives of type 105.

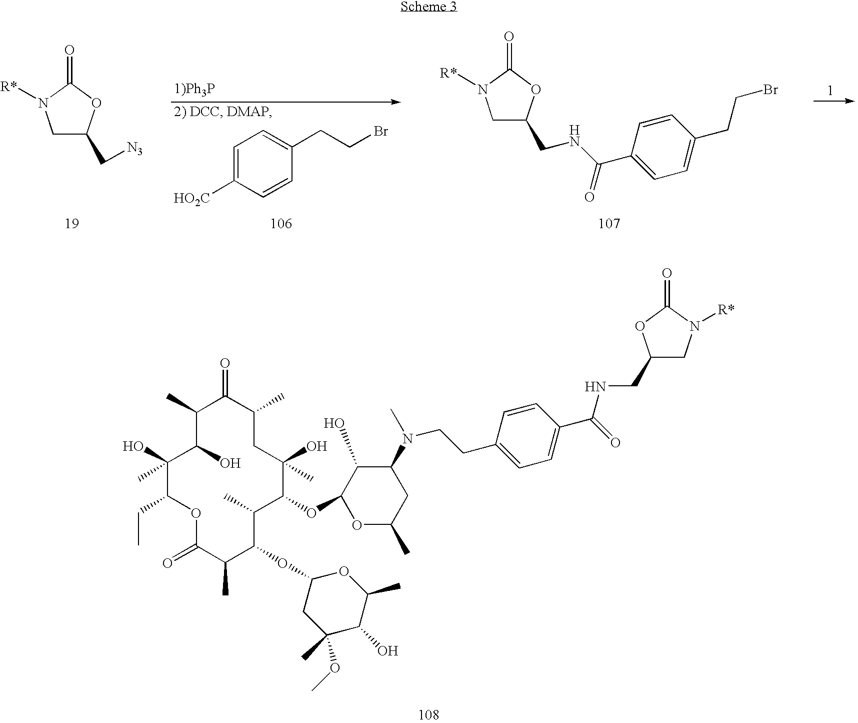

Scheme 3 illustrates the synthesis of amide-linked C-5 benzoyl derivatives. Azide 19 can be reduced to afford the corresponding C-5 amino oxazolidinones. Such amines are readily converted to amides with substituted benzoic acid derivatives similar to 106. The resulting coupling product 107 can then alkylate macrolide derivative 1 to yield target compounds of type 108.

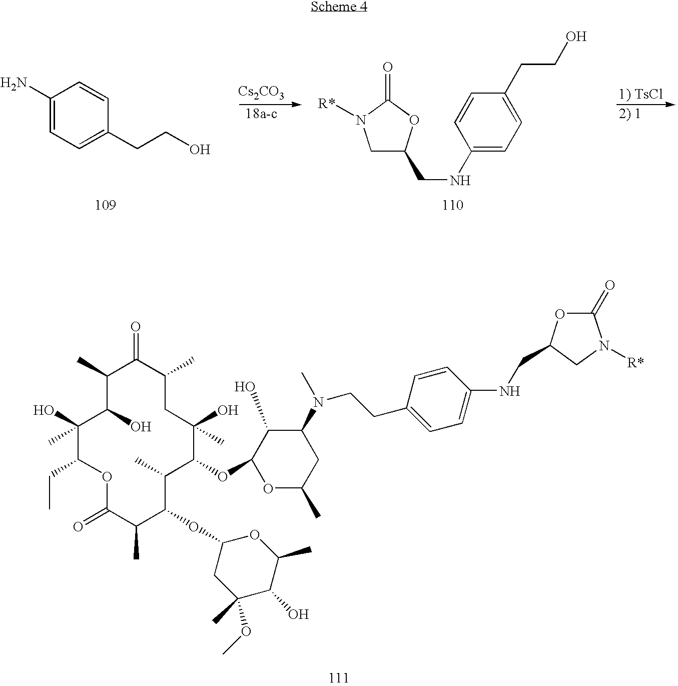

Scheme 4 illustrates the synthesis of C-5 aniline derivatives of type 111. Substituted aniline derivative 109 can be alkylated with oxazolidinones 18a-c to yield compounds of type 110. The resulting primary alcohol may then be converted to a leaving group using procedures similar to those described in Scheme 1, and then alkylated with macrolide derivative 1 to yield the target compounds 111.

In addition to the compounds described in Schemes 1-4 above, one skilled in the art could synthesize compounds that include various other macrolide, linker, and side-chain moieties, including, but not limited to, those compounds listed in Table 1, using chemical reactions known in the art.

4. Characterization of Compounds of the Invention

Compounds designed, selected and/or optimized by methods described above, once produced, may be characterized using a variety of assays known to those skilled in the art to determine whether the compounds have biological activity. For example, the molecules may be characterized by conventional assays, including but not limited to those assays described below, to determine whether they have a predicted activity, binding activity and/or binding specificity.

Furthermore, high-throughput screening may be used to speed up analysis using such assays. As a result, it may be possible to rapidly screen the molecules described herein for activity, for example, as anti-cancer, anti-bacterial, anti-fungal, anti-parasitic or anti-viral agents. Also, it may be possible to assay how the compounds interact with a ribosome or ribosomal subunit and/or are effective as modulators (for example, inhibitors) of protein synthesis using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

(1) Surface Binding Studies. A variety of binding assays may be useful in screening new molecules for their binding activity. One approach includes surface plasmon resonance (SPR) that can be used to evaluate the binding properties of molecules of interest with respect to a ribosome, ribosomal subunit or a fragment thereof.

SPR methodologies measure the interaction between two or more macromolecules in real-time through the generation of a quantum-mechanical surface plasmon. One device, (BIAcore Biosensor RTM from Pharmacia Biosensor, Piscatawy, N.J.) provides a focused beam of polychromatic light to the interface between a gold film (provided as a disposable biosensor “chip”) and a buffer compartment that can be regulated by the user. A 100 nm thick “hydrogel” composed of carboxylated dextran that provides a matrix for the covalent immobilization of analytes of interest is attached to the gold film. When the focused light interacts with the free electron cloud of the gold film, plasmon resonance is enhanced. The resulting reflected light is spectrally depleted in wavelengths that optimally evolved the resonance. By separating the reflected polychromatic light into its component wavelengths (by means of a prism), and determining the frequencies that are depleted, the BIAcore establishes an optical interface which accurately reports the behavior of the generated surface plasmon resonance. When designed as above, the plasmon resonance (and thus the depletion spectrum) is sensitive to mass in the evanescent field (which corresponds roughly to the thickness of the hydrogel). If one component of an interacting pair is immobilized to the hydrogel, and the interacting partner is provided through the buffer compartment, the interaction between the two components can be measured in real time based on the accumulation of mass in the evanescent field and its corresponding effects of the plasmon resonance as measured by the depletion spectrum. This system permits rapid and sensitive real-time measurement of the molecular interactions without the need to label either component.

(2) Fluorescence Polarization. Fluorescence polarization (FP) is a measurement technique that can readily be applied to protein-protein, protein-ligand, or RNA-ligand interactions in order to derive IC₅₀s and Kds of the association reaction between two molecules. In this technique one of the molecules of interest is conjugated with a fluorophore. This is generally the smaller molecule in the system (in this case, the compound of interest). The sample mixture, containing both the ligand-probe conjugate and the ribosome, ribosomal subunit or fragment thereof, is excited with vertically polarized light. Light is absorbed by the probe fluorophores, and re-emitted a short time later. The degree of polarization of the emitted light is measured. Polarization of the emitted light is dependent on several factors, but most importantly on viscosity of the solution and on the apparent molecular weight of the fluorophore. With proper controls, changes in the degree of polarization of the emitted light depends only on changes in the apparent molecular weight of the fluorophore, which in-turn depends on whether the probe-ligand conjugate is free in solution, or is bound to a receptor. Binding assays based on FP have a number of important advantages, including the measurement of IC₅₀s and Kds under true homogenous equilibrium conditions, speed of analysis and amenity to automation, and ability to screen in cloudy suspensions and colored solutions.

(3) Protein Synthesis. It is contemplated that, in addition to characterization by the foregoing biochemical assays, the compound of interest may also be characterized as a modulator (for example, an inhibitor of protein synthesis) of the functional activity of the ribosome or ribosomal subunit.

Furthermore, more specific protein synthesis inhibition assays may be performed by administering the compound to a whole organism, tissue, organ, organelle, cell, a cellular or subcellular extract, or a purified ribosome preparation and observing its pharmacological and inhibitory properties by determining, for example, its inhibition constant (IC₅₀) for inhibiting protein synthesis. Incorporation of ³H leucine or ³⁵S methionine, or similar experiments can be performed to investigate protein synthesis activity. A change in the amount or the rate of protein synthesis in the cell in the presence of a molecule of interest indicates that the molecule is a modulator of protein synthesis. A decrease in the rate or the amount of protein synthesis indicates that the molecule is a inhibitor of protein synthesis.

Furthermore, the compounds may be assayed for anti-proliferative or anti-infective properties on a cellular level. For example, where the target organism is a microorganism, the activity of compounds of interest may be assayed by growing the microorganisms of interest in media either containing or lacking the compound. Growth inhibition may be indicative that the molecule may be acting as a protein synthesis inhibitor. More specifically, the activity of the compounds of interest against bacterial pathogens may be demonstrated by the ability of the compound to inhibit growth of defined strains of human pathogens. For this purpose, a panel of bacterial strains can be assembled to include a variety of target pathogenic species, some containing resistance mechanisms that have been characterized. Use of such a panel of organisms permits the determination of structure-activity relationships not only in regards to potency and spectrum, but also with a view to obviating resistance mechanisms. The assays may be performed in microtiter trays according to conventional methodologies as published by The National Committee for Clinical Laboratory Standards (NCCLS) guidelines (NCCLS. M7-A5-Methods for Dilution Antimicrobial Susceptibility Tests for Bacteria That Grow Aerobically; Approved Standard-Fifth Edition. NCCLS Document M100-S12/M7 (ISBN 1-56238-394-9)).

5. Formulation and Administration

The compounds of the invention may be useful in the prevention or treatment of a variety of human or other animal disorders, including for example, bacterial infection, fungal infections, viral infections, parasitic diseases, and cancer. It is contemplated that, once identified, the active molecules of the invention may be incorporated into any suitable carrier prior to use. The dose of active molecule, mode of administration and use of suitable carrier will depend upon the intended recipient and target organism. The formulations, both for veterinary and for human medical use, of compounds according to the present invention typically include such compounds in association with a pharmaceutically acceptable carrier.

The carrier(s) should be “acceptable” in the sense of being compatible with the other ingredients of the formulations and not deleterious to the recipient. Pharmaceutically acceptable carriers, in this regard, are intended to include any and all solvents, dispersion media, coatings, anti-bacterial and anti-fungal agents, isotonic and absorption delaying agents, and the like, compatible with pharmaceutical administration. The use of such media and agents for pharmaceutically active substances is known in the art. Except insofar as any conventional media or agent is incompatible with the active compound, use thereof in the compositions is contemplated. Supplementary active compounds (identified or designed according to the invention and/or known in the art) also can be incorporated into the compositions. The formulations may conveniently be presented in dosage unit form and may be prepared by any of the methods well known in the art of pharmacy/microbiology. In general, some formulations are prepared by bringing the compound into association with a liquid carrier or a finely divided solid carrier or both, and then, if necessary, shaping the product into the desired formulation.

A pharmaceutical composition of the invention should be formulated to be compatible with its intended route of administration. Examples of routes of administration include oral or parenteral, for example, intravenous, intradermal, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Useful solutions for oral or parenteral administration can be prepared by any of the methods well known in the pharmaceutical art, described, for example, in Remington's Pharmaceutical Sciences, (Gennaro, A., ed.), Mack Pub., (1990). Formulations for parenteral administration can also include glycocholate for buccal administration, methoxysalicylate for rectal administration, or citric acid for vaginal administration. The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions which are solid at room temperature and liquid at body temperatures. Formulations also can include, for example, polyalkylene glycols such as polyethylene glycol, oils of vegetable origin, and hydrogenated naphthalenes. Formulations for direct administration can include glycerol and other compositions of high viscosity. Other potentially useful parenteral carriers for these drugs include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Formulations of the present invention suitable for oral administration may be in the form of: discrete units such as capsules, gelatin capsules, sachets, tablets, troches, or lozenges, each containing a predetermined amount of the drug; a powder or granular composition; a solution or a suspension in an aqueous liquid or non-aqueous liquid; or an oil-in-water emulsion or a water-in-oil emulsion. The drug may also be administered in the form of a bolus, electuary or paste. A tablet may be made by compressing or moulding the drug optionally with one or more accessory ingredients. Compressed tablets may be prepared by compressing, in a suitable machine, the drug in a free-flowing form such as a powder or granules, optionally mixed by a binder, lubricant, inert diluent, surface active or dispersing agent. Moulded tablets may be made by moulding, in a suitable machine, a mixture of the powdered drug and suitable carrier moistened with an inert liquid diluent.

Oral compositions generally include an inert diluent or an edible carrier. For the purpose of oral therapeutic administration, the active compound can be incorporated with excipients. Oral compositions prepared using a fluid carrier for use as a mouthwash include the compound in the fluid carrier and are applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration may be in the form of a sterile aqueous preparation of the drug that may be in microcrystalline form, for example, in the form of an aqueous microcrystalline suspension. Liposomal formulations or biodegradable polymer systems may also be used to present the drug for both intra-articular and ophthalmic administration.

Formulations suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the drug with a dermatologically acceptable carrier such as a lotion, cream, ointment or soap. Particularly useful are carriers capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used.

For inhalation treatments, inhalation of powder (self-propelling or spray formulations) dispensed with a spray can, a nebulizer, or an atomizer can be used. Such formulations can be in the form of a fine powder for pulmonary administration from a powder inhalation device or self-propelling powder-dispensing formulations. In the case of self-propelling solution and spray formulations, the effect may be achieved either by choice of a valve having the desired spray characteristics (ie., being capable of producing a spray having the desired particle size) or by incorporating the active ingredient as a suspended powder in controlled particle size. For administration by inhalation, the compounds also can be delivered in the form of an aerosol spray from pressured container or dispenser which contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents and bile salts. Transmucosal administration can be accomplished through the use of nasal sprays or suppositories. For transdermal administration, the active compounds typically are formulated into ointments, salves, gels, or creams as generally known in the art.

The active compounds may be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art- Liposomal suspensions can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. Furthermore, administration can be by periodic injections of a bolus, or can be made more continuous by intravenous, intramuscular or intraperitoneal administration from an external reservoir (e.g., an intrvenous bag).

Where adhesion to a tissue surface is desired the composition can include the drug dispersed in a fibrinogen-thrombin composition or other bioadhesive. The compound then can be painted, sprayed or otherwise applied to the desired tissue surface. Alternatively, the drugs can be formulated for parenteral or oral administration to humans or other mammals, for example, in therapeutically effective amounts, e.g., amounts that provide appropriate concentrations of the drug to target tissue for a time sufficient to induce the desired effect.

Where the active compound is to be used as part of a transplant procedure, it can be provided to the living tissue or organ to be transplanted prior to removal of tissue or organ from the donor. The compound can be provided to the donor host. Alternatively or, in addition, once removed from the donor, the organ or living tissue can be placed in a preservation solution containing the active compound. In all cases, the active compound can be administered directly to the desired tissue, as by injection to the tissue, or it can be provided systemically, either by oral or parenteral administration, using any of the methods and formulations described herein and/or known in the art Where the drug comprises part of a tissue or organ preservation solution, any commercially available preservation solution can be used to advantage. For example, useful solutions known in the art include Collins solution, Wisconsin solution, Belzer solution, Eurocollins solution and lactated Ringer's solution.

Active compound as identified or designed by the methods described herein can be administered to individuals to treat disorders (prophylactically or therapeutically). In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a drug as well as tailoring the dosage and/or therapeutic regimen of treatment with the drug.

In therapeutic use for treating, or combating, bacterial infections in mammals, the compounds or pharmaceutical compositions thereof will be administered orally, parenterally and/or topically at a dosage to obtain and maintain a concentration, that is, an amount, or blood-level or tissue level of active component in the animal undergoing treatment which will be anti-microbially effective. The term “effective amount” is understood to mean that the compound of the invention is present in or on the recipient in an amount sufficient to elicit biological activity, for example, anti-microbial activity, anti-fungal activity, anti-viral activity, anti-parasitic activity, and/or anti-proliferative activity. Generally, an effective amount of dosage of active component will be in the range of from about 0.1 to about 100, more preferably from about 1.0 to about 50 mg/kg of body weight/day. The amount administered will also likely depend on such variables as the type and extent of disease or indication to be treated, the overall health status of the particular patient, the relative biological efficacy of the compound delivered, the formulation of the drug, the presence and types of excipients in the formulation, and the route of administration. Also, it is to be understood that the initial dosage administered may be increased beyond the above upper level in order to rapidly achieve the desired blood-level or tissue level, or the initial dosage may be smaller than the optimum and the daily dosage may be progressively increased during the course of treatment depending on the particular situation. If desired, the daily dose may also be divided into multiple doses for administration, for example, two to four times per day.

### EXAMPLES

In the following examples, nuclear magnetic resonance (NMR) spectra were obtained on a Bruker Avance 300 or Avance 500 spectrometer, or in some cases a GE-Nicolet 300 spectrometer. Common reaction solvents were either high performance liquid chromatography (HPLC) grade or American Chemical Society (ACS) grade, and anhydrous as obtained from the manufacturer unless otherwise noted. “Chromatography” or “purified by silica gel” refers to flash column chromatography using silica gel (EM Merck, Silica Gel 60, 230-400 mesh) unless otherwise noted.

### Example 1

### Exemplary Compounds

Exemplary compounds synthesized in accordance with the invention are listed in Table 1.

TABLE 1

| Compound Number | Structure |
|---|---|
| 40 | 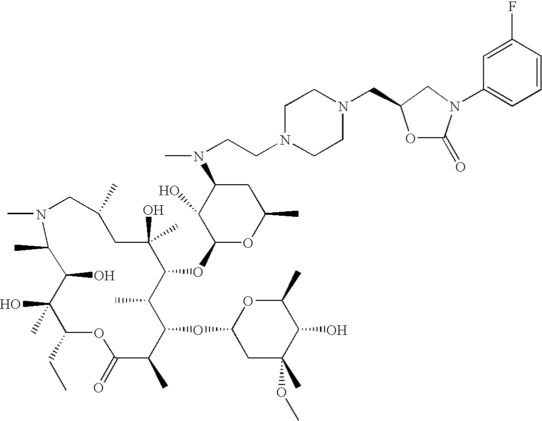 |
| 41 | 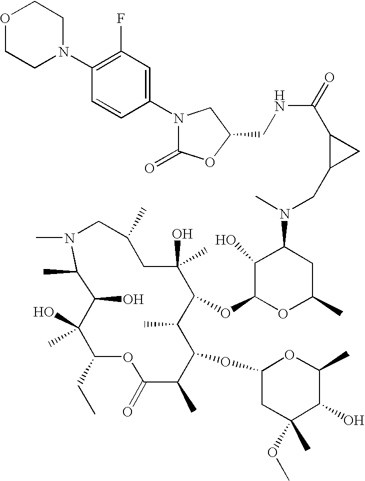 |

### Example 2

### Synthesis of Compound 40

Scheme 5 illustrates the synthesis of compound 40. Mesylate 48 served as the alkylating agent for 1-(2-hydroxyethyl)piperazine to afford alcohol 49. Mesylation of 49 gave mesylate 50, which was used to alkylate amine 2 to yield compound 40. 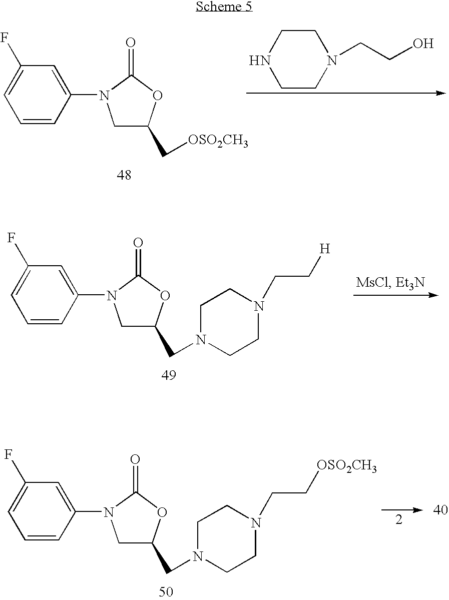
Synthesis of Alcohol 49

A solution of mesylate 48 (500 mg, 1.72 mmol; synthesized from 3-fluoroaniline using chemistry reported in the literature (Brickner, S. J. et al. J. Med. Chem. 1996, 39, 673)) in N-methylpyrrolidone (5 mL) was treated with 1-(2-hydroxyethyl)piperazine (225 mg, 1.73 mmol), and N,N-diisopropylethylamine (Hunig's base or i-Pr₂NEt, 0.3 mL, 1.73 mmol) and the mixture was heated to 95° C. for 3 hours (hr). The reaction mixture was cooled to room temperature, diluted with ethyl acetate EtOAc, 30 mL), and washed with water (H₂O, 3 mL). The aqueous layer was treated with 2 g of sodium chloride (NaCl), then 20 mL of chloroform was added and the mixture was stirred at room temperature for 30 minutes (min). The combined organic phase was dried over sodium sulfate (Na₂SO₄) and concentrated in vacuo. The residue was purified by flash column chromatography over silica gel (eluting with 90% methylene chloride (CH₂Cl₂), 10% methanol (MeOH), 0.1% ammonium hydroxide) to provide alcohol 49 (400 mg, 80% yield) as a white solid. LCMS (ESI) m/z 324 (M+H)⁺; ¹HNMR (300 MHz, CDCl₃; partial): δ 7.33 (d, J=3 Hz, 1H), 7.23 (m, 2H), 6.86 (m, 1H), 4.78 (m, 1H), 4.06 (t, J=9, 18 Hz, 1H), 3.80 (t, J=9, 15 Hz, 1H), 3.62 (m, 2H), 3.40 (t, J=3, 6 Hz, 1H),3.37 (m, 4H), 2.81 (m, 6H).

Synthesis of Mesylate 50

Alcohol 49 (0.150 g, 0.46 mmol) was dissolved in 2 mL CH₂Cl₂, and the mixture was cooled to 0° C. Triethylamine (Et₃N, 3.0 mL, 21.37 mmol) was added, followed by methanesulfonyl chloride (MsCl) (0.04 mL, 0.55 mmol). The mixture was allowed to warm to room temperature and stirred for 2 hr. CH₂Cl₂ (20 mL) was added, and the mixture washed twice with 1 N hydrochloric acid (HCl), twice with 10% aqueous sodium carbonate Na₂CO₃), and then brine. The organic phase was dried over Na₂SO₄, and evaporated to yield mesylate 50 (0.170 g, 91% yield) as a white solid. LCMS (ESI) m/z 402 (M+H)⁺.

Synthesis of Compound 40

A solution of amine 2 (0.200 g, 0.270 mmol) in acetonitrile (CH₃CN, 10 mL) was treated with mesylate 50 (0.210 mg, 0.54 mmol) and Hunig's base (0.07 mL, 0.41 mmol). The reaction mixture was heated to 80° C. for 3 hr and allowed to cool to room temperature. CH₃CN was removed in vacuo, and the residue was purified by preparative thin layer chromatography (using 90% CH₂Cl₂, 10% MeOH, 0.1% NH₄OH as eluant) to provide compound 40 as a white solid. LCMS (ESI) m/z 1041 (M+H)⁺; ¹HNMR (300 MHz, CDCl₃; partial): δ 9.46 (s, 1H), 7.34 (dd, J=3, 12 Hz, 1H), 7.22 (m, 2H), 6.77 (t, J=3,3 Hz 1H), 5.01 (m, 2H), 4.62 (m, 2H), 4.34 (d, J=2 Hz, 1H), 3.98 (m, 1H), 3.55 (m, 1H), 3.27 (m, 1H), 3.41 (s, 1H), 3.27 (s, 1H), 1.20 (m, 2H), 1.10 (m, 1H), 0.98 (m, 3H), 0.95 (nm, 3H).

### Example 3

### Synthesis of Compound 41

Scheme 6 illustrates the synthesis of compound 41. Known amine 3 (for a synthesis see: Brickner et al., J. Med Chem., vol. 39, p. 673 (1996)) was condensed with trans-2-formyl-1-cyclopropanecarboxylic acid 52 (prepared from the saponification of the commercially available ethyl ester) to form aldehyde 51, which was used to alkylate amine 2 to yield compound 41. 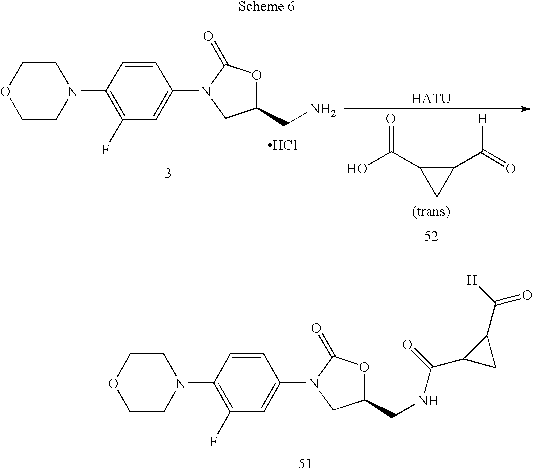 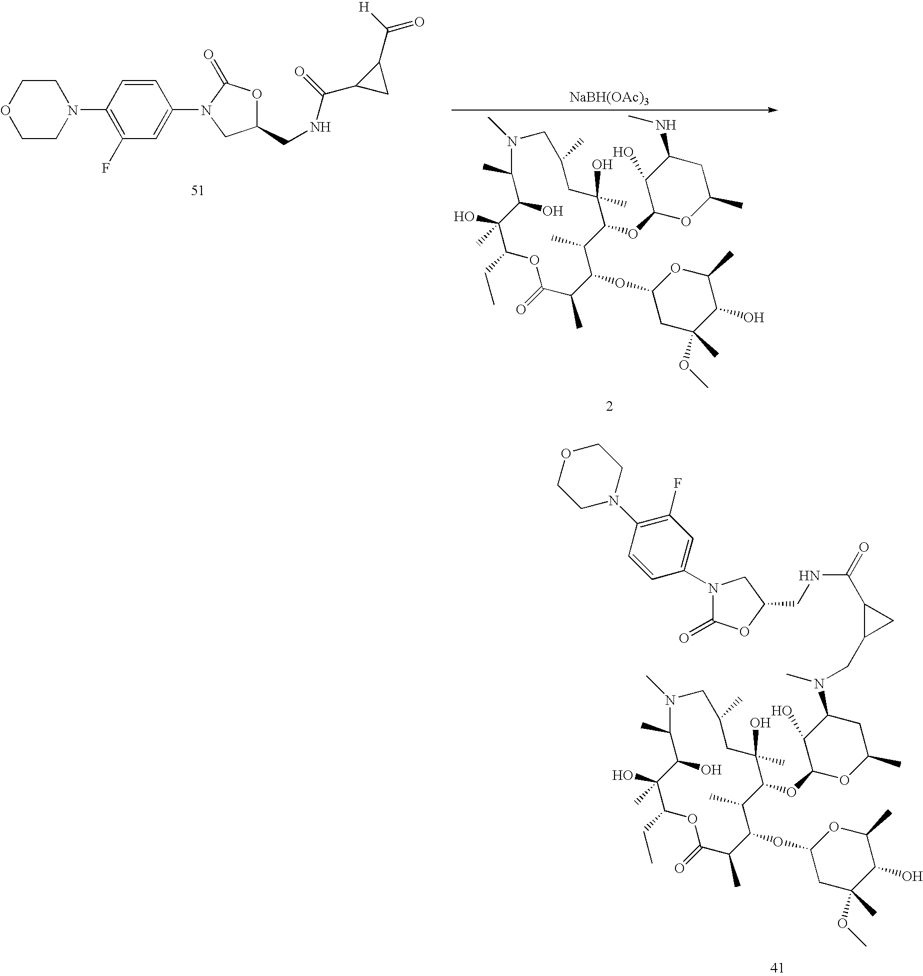
Synthesis of Carboxylic Acid 52

A solution of ethyl trans-2-formyl-1-cyclopropanecarboxylate (2.0 mL, 14 mmol) in MeOH (30 mL) was treated with 1.0 M aqueous NaOH (30 mL, 30 mmol) and stirred for 30 min at 23° C. The reaction mixture was quenched by the addition of 6 M HCl (10 mL, 60 mmol) and extracted with CH₂Cl₂ (3×50 mL). Drying (Na2SO₄) and evaporation provided trans-2-formyl-1-cyclopropanecarboxylic acid (carboxylic acid 52) as a white crystalline solid (1.5 g, 88%).

Synthesis of Aldehyde 51

A solution of carboxylic acid 52 (0.68 g, 0.60 mmol) in CH₂Cl₂ (5 mL) was treated with amine 3 (0.14 g, 0.42 mmol), Hunig's base (0.22 mL, 1.3 mmol) and O-(7-azabenzotriazol-1-yl)-1,1,3,3-tetratethyluronium hexafluorophosphate (HATU, 0.19 g, 0.50 mmol) and stirred at 23° C. for 2 hr. The reaction mixture was diluted with H₂O (30 mL) and extracted with CH₂Cl₂ (3×30 mL), dried (Na₂SO₄), and evaporated. Flash chromatography (SiO₂, 10% MeOH, 45% CH₂Cl₂, 45% EtOAc) provided aldehyde 51 (100 mg, 61%) as a 1:1 mixture of diastereomers: LCMS (ESI) m/e 392 (M+H)⁺.

Synthesis of Compound 41

A solution of aldehyde 51 (8.0 mg, 0.020 mmol) in dimethylformamide (0.25 mL) was treated with amine 2 (4.5 mg, 0.0067 mmol), acetic acid (0.006 mL) and sodium triacetoxyborohydride (NaBH(OAc)₃, 5.6 mg, 0.027 mmol), and stirred at 23° C. for 2 hr. The reaction mixture was evaporated to a white residue that was purified by preparative thin layer chromatography (SiO₂, 1% ammonia, 10% MeOH, 89% CH₂Cl₂) to afford compound 41 (3.5 mg, 46%) as a colorless film: LCMS (ESI) m/e 556 (M+2H)²⁺; ¹H NMR (300 MHz, deuterated methanol, partial): δ 7.52-7.46 (m, 1H), 7.18-7.13 (m, 1H), 7.08-7.01 (m, 1H), 5.03 (d, J=5 Hz, 1 H), 4.75, (m, 1H), 4.55 (m, 1H), 1.06 (d, J =10 Hz, 3 H), 0.98 (d, J=10 Hz, 3 H), 0.89 (t, J=7 Hz, 3H).

Incorporation by Reference

The entire disclosure of each of the patent documents and scientific articles referred to herein is incorporated by reference for all purposes.

Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A compound having the formula: 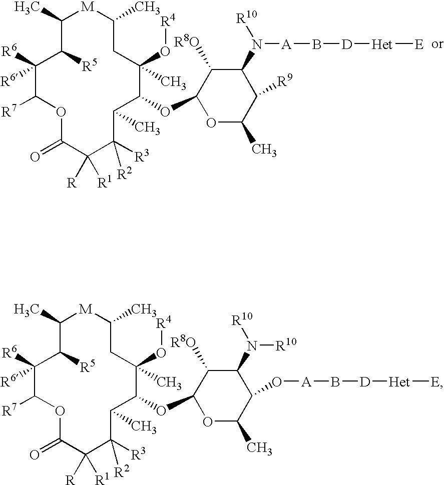
or pharmaceutically acceptable salt, ester or prodrug thereof,
wherein:
D-Het is selected from the group consisting of: 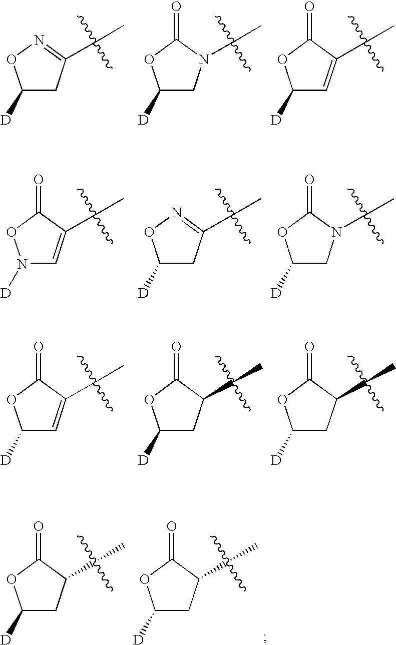
A is selected from the group consisting of:
a) C₁₋₆ acyl, b) C₁₋₆ alkyl, c) C₂₋₆ alkenyl, and d) —C(O)-C₂₋₆ alkenyl,
wherein
i) one or more carbon atoms of any of a)-d) optionally is replaced by a moiety selected from the group consisting of O, S(O)ₚ, and NR¹¹, and
ii) any of a)-d) optionally is substituted with one or more R¹² groups;
B is selected from the group consisting of:
a) 3-4 membered saturated or unsaturated heterocycle containing one or more heteroatoms selected from the group consisting of nitrogen, oxygen, and sulfur, b) 6-10 membered saturated, unsaturated, or aromatic heterocycle containing one or more heteroatoms selected from the group consisting of nitrogen, oxygen, and sulfur, c) 3-4 membered saturated, unsaturated, or aromatic carbocycle, d) 6-10 membered saturated, unsaturated, or aromatic carbocycle,
wherein any of a)-d) optionally is substituted with one or more R¹² groups;
B-D is selected from the group consisting of:
a) B-C₁₋₆ alkyl, b) B-C₂₋₆ alkenyl, c) B-C₁₋₆ alkynyl, d) B—O—C₁₋₆ alkyl e) B—O—C₁₋₆ alkenyl, f) B—O—C₁₋₆ alkynyl, g) B—NR¹¹-C₁₋₆ alkyl, h) B—NR¹¹-C₁₋₆ alkenyl, i) B—NR¹¹-C₁₋₆ alkynyl, j) B—S(O)ₚ-C₁₋₆ alkyl, k) B—S(O)ₚ-C₁₋₆ alkenyl, and l) B—S(O)ₚ-C₁₋₆ alkynyl,
wherein any of a)-l) optionally is substituted with one or more moieties
selected from the group consisting of ═O, ═S, ═NOR¹¹, and R¹²;
E is selected from the group consisting of: 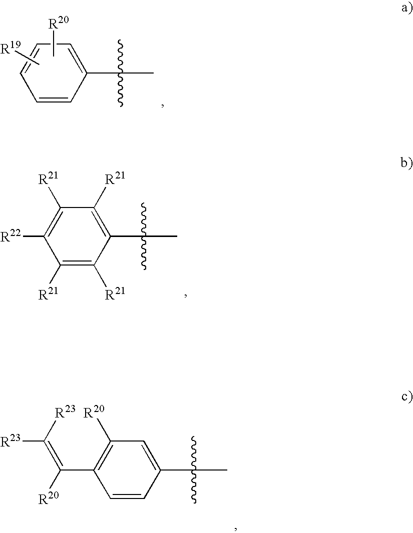
d) 5-10 membered saturated, unsaturated, or aromatic heterocycle containing one or more heteroatoms selected from the group consisting of nitrogen, oxygen, and sulfur, and optionally substituted with one or more R¹² groups,
e) C₅₋₁₀ saturated, unsaturated, or aromatic carbocycle, optionally substituted with one or more R¹² groups,
f) C₁₋₈ alkyl,
g) C₂₋₈ alkenyl,
h) C₂₋₈ alkynyl,
i) C₁₋₈ alkoxy,
j) C₁₋₈ alkylthio,
k) C₁₋₈ acyl,
l) S(O)ₚR¹¹, and
m) hydrogen,
wherein any of f)-k) optionally is substituted with
i) one or more R¹² groups,
ii) 5-6 membered saturated, unsaturated, or aromatic heterocycle containing one or more heteroatoms selected from the group consisting of nitrogen, oxygen, and sulfur, and optionally substituted with one or more R¹² groups, or
iii) C₅₋₁₀ saturated, unsaturated, or aromatic carbocycle, optionally substituted with one or more R¹² groups;
M is selected from the group consisting of:
a) —C(O)—, b) —C(═NOR¹¹)—, c) —CH(—OR¹¹)—, d) —NR¹¹—CH₂—, e) —CH₂—NR¹¹—, f) —CH(NR¹¹R¹¹)—, g) —C(═NNR¹¹R¹¹)—, h) —NR¹¹—C(O)—, i) —C(O)NR¹¹—, and j) —C(═NR¹¹)—;
R is selected from the group consisting of H and C₁₋₆ alkyl;
R¹ is selected from the group consisting of:
a) H, b) Cl, c) F, d) Br, e) I, f) —NR¹¹R¹¹, g) —NR¹¹C(O)R¹¹, h) —OR¹¹, i) —OC(O)R¹¹, j) —OC(O)OR¹¹, k) —OC(O)NR¹¹R¹¹, l) C₁₋₆ alkyl, m) —OC(O)—C₁₋₆ alkyl, n) —OC(O)O₁₋₆ alkyl, o) —OC(O)NR¹¹-C₁₋₆ alkyl, p) C₁₋₆ alkyl, q) C₁₋₆ alkenyl, r) C₁₋₆ alkynyl,
wherein any of l)-r) optionally is substituted with one or more R¹² groups;
R² is H;
R³ is selected from the group consisting of:
a) H, b) —OR¹¹, c) —O—C₁₋₆ alkyl-R¹², d) —OC(O)R¹¹, e) —OC(O)—C₁₋₆ alkyl-R¹², f) —OC(O)OR¹¹, g) —OC(O)O—C₁₋₆ alkyl-R¹², h) —OC(O)NR¹¹R¹¹, i) —OC(O)NR¹¹-C₁₋₆ alkyl-R¹², and j) 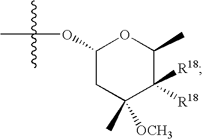
alternatively, R² and R³ taken together form a carbonyl group;
R⁴ is selected from the group consisting of:
a) H, b) R¹¹, c) —C(O)R¹¹d) —C(O)OR¹¹ e) —C(O)NR¹¹R¹¹, f) -C₁₋₆ alkyl-G-R¹¹, g) -C₂₋₆ alkenyl-G-R¹¹, and h) -C₂₋₆ alkynyl-G-R¹¹;
alternatively R³ and R⁴, taken together with the atoms to which they are bonded, form: 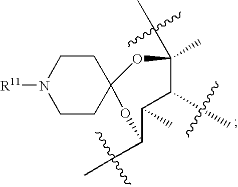
G is selected from the group consisting of:
a) —C(O)—, b) —C(O)O—, c) —C(O)NR¹¹—, d) —C(═NR¹¹)—, e) —C(═NR¹¹)O—, f) —C(═NR¹¹)NR¹¹—, g) —OC(O)—, h) —OC(O)O—, i) —OC(O)NR¹¹—, j) —NR¹¹C(O), k) —NR¹¹C(O)O—, l) —NR¹¹C(O)NR¹¹—, m) —NR¹¹C(═NR¹¹)NR¹¹—, and o) —S(O)ₚ—;
R⁵ is selected from the group consisting of:
a) R¹¹, b) —OR¹¹, c) —NR¹¹R¹¹, d) C₁₋₆ alkyl-R¹², e) —C(O)-R¹¹, f) —(O)-C₁₋₆ alkyl-R¹², g) —OC(O)R¹¹, h) —OC(O—C₁₋₆ alkyl-R¹², i) —OC(O)O-R¹¹, j) —OC(O)O—C₁₋₆ alkyl-R¹², k) —OC(O)NR¹¹R¹¹, l) —OC(O)NR¹¹-C₁₋₆ alkyl-R¹², m) —C(O)-C₂₋₆ alkenyl-R¹², and n) —C(O)-C₂₋₆ alkynyl-R¹²;
alternatively, R⁴ and R⁵, taken together with the atoms to which they are bonded, form: 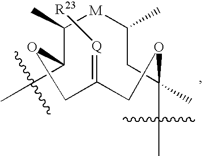
wherein
Q is CH or N, and
R²³ is —OR¹¹, or R¹¹;
R⁶ is selected from the group consisting of:
a) —OR¹¹, b) -C₁₋₆ alkoxy-R¹², c) —C(O)R¹¹, d) OC(O)R¹¹, e) —OC(O)OR¹¹, f) —OC(O)NR¹¹R¹¹, and g) —NR¹¹R¹¹,
alternatively, R⁵ and R⁶ taken together with the atoms to which they are attached form a 5-membered ring by attachment to each other through a linker selected from the group consisting of:
a) —OC(R¹²)₂O—, b) —OC(O)O—, c) —OC(O)NR¹¹—, d) —NR¹¹C(O)O—, e) —OC(O)NOR¹¹—, f) —NOR¹¹—C(O)O—, g) OC(O)NNR¹¹R¹¹—, h) —NNR¹¹R¹¹—C(O)O—, i) —OC(O)C(R¹²)₂—, j) —C(R¹²)₂C(O)O—, k) —OC(S)O—, l) —OC(S)NR¹¹, m) —NR¹¹C(S)O—, n) —OC(S)NOR¹¹—, o) —NOR¹¹—C(S)O—, p) —OC(S)NNR¹¹R¹¹—, q) —NNR¹¹R¹¹—C(S)O—, r) —OC(S)C(R¹²)₂—, and s) —C(R¹²)₂C(S)O—;
alternatively, M, R⁵, and R⁶ taken together with the atoms to which they are attached form: 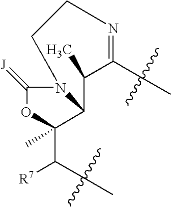
wherein J is selected from the group consisting of O and NR¹¹;
R⁶′ is selected from the group consisting of
a) H, b) -C₁₋₄ alkyl, c) -C₂₋₄ alkenyl, which can be further substituted with C₁₋₁₂ alkyl or one or more halogens, d) -C₂₋₄ alkynyl, which can be further substituted with C₁₋₁₂ alkyl or one or more halogens, e) aryl or heteroaryl, which can be further substituted with C₁₋₁₂ alkyl or one or more halogens, f) —C(O)H, g) —COOH, h) —CN, i) —COOR¹¹, j) —C(O)NR¹¹R¹¹, k) —C(O)R¹¹, and l) —C(O)SR¹¹, wherein b) is further substituted with one or more substituents selected from the group consisting of aa) —OR¹¹, bb) halogen, cc) —SR¹¹, dd) C₁₋₁₂ alkyl, which can be further substituted with halogen, hydroxyl, C₁₋₆alkoxy, or amino, ee) —OR¹¹, ff) —SR¹¹, gg) —NR¹¹R¹¹, hh) —CN, ii) —NO₂, jj) —NC(O)R¹¹, kk) —COOR¹¹, ll) —N₃, mm) ═N—O-R¹¹, nn) ′NR¹¹, oo) ═N—NR¹¹R¹¹, pp) ═N—NH—C(O)R¹¹, and qq) ═N—NH—C(O)NR¹¹R¹¹;
alternatively R⁶ and R^{6′} are taken together with the atom to which they are attached to form an epoxide, a carbonyl, an olefin, or a substituted olefin, or a C₃-C₇ carbocyclic, carbonate, or carbamate, wherein the nitrogen of said carbamate can be further substituted with a C₁-C₆ alkyl;
R⁷ is selected from the group consisting of:
a) C₁₋₆ alkyl, b) C₂₋₆ alkenyl, and c) C₂₋₆ alkynyl,
wherein any of a)-c) optionally is substituted with one or more R¹² groups;
R⁸ is selected from the group consisting of H and —C(O)R¹¹;
R⁹ is selected from the group consisting of H, OH, and OR¹¹;
R¹⁰ is selected from the group consisting of:
a) H, b) R¹¹, c) -C₁₋₆ alkyl-G-R¹¹, d) -C₂₋₆ alkenyl-G-R¹¹, and e) -C₂₋₆ alkynyl-G-R¹¹,
wherein any of c)-e) optionally is substituted with one or more R₁₂ groups;
R¹¹, at each occurrence, independently is selected from the group consisting of:
a) H, b) C₁₋₆ alkyl, c) C₂₋₆ alkenyl, d) C₂₋₆ alkynyl, e) C₆₋₁₀ saturated, unsaturated, or aromatic carbocycle, f) 3-12 membered saturated, unsaturated, or aromatic heterocycle containing one or more heteroatoms selected from the group consisting of nitrogen, oxygen, and sulfur, g) —C(O)—C₁₋₆ alkyl, h) —C(O)C₂₋₆ alkenyl, i) —C(O)—C₂₋₆ alkynyl, j) —C(O)—C₆₋₁₀ saturated, unsaturated, or aromatic carbocycle, k) —C(O)3-12 membered saturated, unsaturated, or aromatic heterocycle containing one or more heteroatoms selected from the group consisting of nitrogen, oxygen, and sulfur, l) —C(O)-C₁₋₆ alkyl, m) —C(O)O—C₂₋₆ alkenyl, n) —C(O)O—C₂₋₆ alkynyl, o) —C(O)O—C₆₋₁₀ saturated, unsaturated, or aromatic carbocycle, p) —C(O)O-3-12 membered saturated, unsaturated, or aromatic heterocycle containing one or more heteroatoms selected from the group consisting of nitrogen, oxygen, and sulfur, and q) —C(O)NR¹³R¹³,
wherein any of b)-p) optionally is substituted with one or more R¹² groups,
alternatively, NR¹¹R¹¹ forms a 3-7 membered saturated, unsaturated or aromatic ring including the nitrogen atom to which the R¹¹ groups are bonded and optionally one or more moieties selected from the group consisting of O, S(O)ₚ, N, and NR¹⁵;
R¹² is selected from the group consisting of:
a) R¹⁴, b) C₂₋₈ alkyl, c) C₂₋₈ alkenyl, d) C₂₋₈ alkynyl, e) C₃₋₁₂ saturated, unsaturated, or aromatic carbocycle, f) 3-12 membered saturated, unsaturated, or aromatic heterocycle containing one or more heteroatoms selected from the group consisting of nitrogen, oxygen, and sulfur, and g) —NR¹⁵C(O)OR¹⁵,
wherein any of b)-f) optionally is substituted with one or more R¹⁴ groups;
R¹³, at each occurrence, independently is selected from the group consisting of:
a) H, b) C₁₋₆ alkyl, c) C₂₋₆ alkenyl, d) C₂₋₆ alkynyl, e) C₃₋₁₀ saturated, unsaturated, or aromatic carbocycle, and f) 3-10 membered saturated, unsaturated, or aromatic heterocycle containing one or more heteroatoms selected from the group consisting of nitrogen, oxygen, and sulfur,
wherein any of b)-f) optionally is substituted with one or more moieties selected from the group consisting of:
aa) carbonyl, bb) formyl, cc) F, dd) Cl, ee) Br, ff) I, gg) CN, hh) NO₂, ii) OR⁵, jj) —S(O)ₚR¹⁵, kk) —C(O)R¹⁵, ll) —C(O)OR¹⁵, mm) —OC(O)R¹⁵, nn) —C(O)NR¹⁵R¹⁵, oo) —OC(O)NR¹⁵R¹⁵, pp) —C(═NR¹⁵)R¹⁵, qq) —C(R¹⁵)(R¹⁵)OR¹⁵, rr) —C(R¹⁵)₂OC(O)R¹⁵, ss) —C(R¹⁵)(OR¹⁵)(CH₂)ᵣNR¹⁵R¹⁵, tt) —NR¹⁵R¹⁵; uu) —NR¹⁵OR¹⁵; vv) —NR¹⁵C(O)R¹⁵, ww) —NR¹⁵C(O)OR¹⁵, xx) —NR¹⁵C(O)NR¹⁵R¹⁵, yy) —NR¹⁵S(O)ᵣR¹⁵, zz) —C(OR¹⁵)(OR¹⁵)R¹⁵, ab) —C(R¹⁵)₂NR¹⁵R¹⁵, ac) ═NR¹⁵, ad) —C(S)NR¹⁵R¹⁵, ae) —NR¹⁵C(S)R¹⁵, af) —OC(S)NR¹⁵R¹⁵, ag) —NR¹⁵C(S)OR¹⁵, ah) —NR¹⁵C(S)NR¹⁵R¹⁵, ai) —SC(O)R¹⁵, aj) C₁₋₈ alkyl, ak) C₂₋₈ alkenyl, al) C₂₋₈ alkynyl, am) C₁₋₈ alkoxy, an) C₁₋₈ alkylthio, ao) C₁₋₈ acyl, ap) saturated, unsaturated, or aromatic C₃₋₁₀ carbocycle, and aq) saturated, unsaturated, or aromatic 3-10 membered heterocycle containing one or more heteroatoms selected from the group consisting of nitrogen, oxygen, and sulfur,
alternatively, NR¹³R¹³ forms a 3-10 membered saturated, unsaturated or aromatic ring including the nitrogen atom to which the R¹³ groups are attached and optionally one or more moieties selected from the group consisting of O, S(O)ₚ, N, and NR¹⁵;
alternatively, CR¹³R¹³ forms a carbonyl group;
R¹⁴, at each occurrence, is selected from the group consisting of:
a) H, b) ═O, c) F, d) Cl, e) Br, f) I, g) (CR¹³R¹³)ᵣCF₃, h) (CR¹³R¹³)ᵣCN, i) (CR¹³R¹³)ᵣNO₂, j) (CR¹³R¹³)ᵣNR¹³(CR¹³R¹³)ₜR¹⁶, k) (CR¹³R¹³)ᵣOR¹⁶, l) (CR¹³R¹³)ᵣS(O)ₚ(CR¹³R¹³)ₜR¹⁶, m) (CR¹³R¹³)ᵣC(O)(CR¹³R¹³)ₜR¹⁶, n) (CR¹³R¹³)ᵣOC(O)(CR¹³R¹³)ₜR¹⁶, o) (CR¹³R¹³)ᵣSC(O)(CR¹³R¹³)ₜR¹⁶, p) (CR¹³R¹³)ᵣC(O)O(CR¹³R¹³)ₜR¹⁶, q) (CR¹³R¹³)ᵣNR¹³C(O)(CR¹³R¹³)ₜR¹⁶, r) (CR¹³R¹³)ᵣC(O)NR¹³(CR¹³R¹³)ₜR¹⁶, s) (CR¹³R¹³)ᵣC(═NR¹³)(CR¹³R¹³)ₜR¹⁶, t) (CR¹³R¹³)ᵣC(═NNR¹³R¹³)(CR¹³R¹³)ₜR¹⁶, u) (CR¹³R¹³)ᵣC(═NNR¹³C(O)R¹³)(CR¹³R¹³)ₜR¹⁶, v) (CR¹³R¹³)ᵣC(═NOR¹⁶)(CR¹³R¹³)ₜR¹⁶, w) (CR¹³R¹³)ᵣNR¹³C(O)O(CR¹³R¹³)ₜR¹⁶, x) (CR¹³R¹³)ᵣOC(O)NR¹³(CR¹³R¹³)ₜR¹⁶, y) (CR¹³R¹³)ᵣNR¹³C(O)NR¹³(CR¹³R¹³)ₜR¹⁶, z) (CR¹³R¹³)ᵣNR¹³S(O)ₚ(CR¹³R¹³)ₜR¹⁶, aa) (CR¹³R¹³)ᵣS(O)ₚNR¹³(CR¹³R¹³)ₜR¹⁶, bb) (CR¹³R¹³)ᵣNR¹³S(O)ₚNR¹³(CR¹³R¹³)ₜR¹⁶, cc) (CR¹³R¹³)ᵣNR¹³R¹³, dd) C₁₋₆ alkyl, ee) C₂₋₆ alkenyl, ff) C₂₋₆ alkynyl, gg) (CR¹³R¹³)ᵣ-3-10 saturated, unsaturated, or aromatic carbocycle, and hh) (CR¹³R¹³),3-10 membered saturated, unsaturated, or aromatic heterocycle containing one or more heteroatoms selected from the group consisting of nitrogen, oxygen, and sulfur,
wherein any of dd)-hh) optionally is substituted with one or more R¹⁶ groups;
alternatively, two R¹⁴ groups may form —(CH₂)ᵤO—;
R¹⁵ is selected from the group consisting of:
a) H, b) C₁₋₆ alkyl, c) C₂₋₆ alkenyl, d) C₂₋₆ alkynyl, e) C₃₋₁₀ saturated, unsaturated, or aromatic carbocycle, f) 3-10 membered saturated, unsaturated, or aromatic heterocycle containing one or more heteroatoms selected from the group consisting of nitrogen, oxygen, and sulfur, g) —C(O)-C₁₋₆ alkyl, h) —C(O)C₁₋₆ alkenyl, g) —C(O)-C₁₋₆ alkynyl, i) —C(O)-C₃₋₁₀ saturated, unsaturated, or aromatic carbocycle, and j) —C(O)-3-10 membered saturated, unsaturated, or aromatic heterocycle containing one or more heteroatoms selected from the group consisting of nitrogen, oxygen, and sulfur,
wherein any of b)-j) optionally is substituted with one or more moieties selected from the group consisting of: aa) H, bb) F, cc) Cl, dd) Br, ee) I, ff) CN, gg) NO₂, hh) OH, ii) NH₂, jj) NH(C₁₋₆ alkyl), kk) N(C₁₋₆ alkyl)₂, ll) C₁₋₆ alkoxy, mm) aryl, nn) substituted aryl, oo) heteroaryl, pp) substituted heteroaryl, and qq) C₁₋₆ alkyl, optionally substituted with one or more moieties selected from the group consisting of aryl, substituted aryl, heteroaryl, substituted heteroaryl, F, Cl, Br, I, CN, NO₂, and OH;
R¹⁶, at each occurrence, independently is selected from the group consisting of:
a) R¹⁷, b) C₁₋₆ alkyl, c) C₂₋₆ alkenyl, d) C₂₋₆ alkynyl, e) C₃₋₁₀ saturated, unsaturated, or aromatic carbocycle, and f) 3-10 membered saturated, unsaturated, or aromatic heterocycle containing one or more heteroatoms selected from the group consisting of nitrogen, oxygen, and sulfur,
wherein any of b)-f) optionally is substituted with one or more R¹⁷ groups;
R¹⁷, at each occurrence, independently is selected from the group consisting of:
a) H, b) ═O, c) F, d) Cl, e) Br, f) I, g) (CR¹³R¹³)ᵣCF₃, h) (CR¹³R¹³)ᵣCN, i) (CR¹³R¹³)ᵣNO₂, j) (CR¹³R¹³)ᵣNR¹³R¹³, k) (CR¹³R¹³)ᵣOR¹¹, l) (CR¹³R¹³)ᵣS(O)ₚR¹³, m) (CR¹³R¹³)ᵣC(O)R¹³, n) (CR¹³R¹³)ᵣC(O)OR¹³, o) (CR¹³R¹³)ᵣOC(O)R¹³, p) (CR¹³R¹³)ᵣNR¹³C(O)R¹³, q) (CR¹³R¹³)ᵣC(O)NR¹³R¹³, r) (CR¹³R¹³)ᵣC(═NR¹³)R¹³, s) (CR¹³R¹³)ᵣNR¹³C(O)NR¹³R¹³, t) (CR¹³R¹³)ᵣNR¹³S(O)ₚR¹³, u) (CR¹³R¹³)ᵣS(O)ₚNR¹³R¹³, v) (CR¹³R¹³)ᵣNR¹³S(O)ₚNR¹³R¹³, w) C₁₋₆ alkyl, x) C₂₋₆ alkenyl, y) C₂₋₆ alkynyl, z) (CR¹³R¹³)ᵣ-C₃₋₁₀ saturated, unsaturated, or aromatic carbocycle, and aa) (CR¹³R¹³)ᵣ-3-10 membered saturated, unsaturated, or aromatic heterocycle containing one or more heteroatoms selected from the group consisting of nitrogen, oxygen, and sulfur,
wherein any of w)-aa) optionally is substituted with one or more moieties selected from the group consisting of R¹³, F, Cl, Br, I, CN, NO₂, —OR¹³, —NH₂, —NH(C₁₋₆ alkyl), —N(C₁₋₆ alkyl)₂, C₁₋₆ alkoxy, C₁₋₆ alkylthio, and C₁₋₆ acyl;
R¹⁸, at each occurrence, independently is selected from the group consisting of:
a) H, b) —OR¹⁵, c) —O—C₁₋₆ alkyl-OC(O)R¹⁵, d) —O—C₁₋₆ alkyl-OC(O)OR¹⁵, e) —O—C₁₋₆ alkyl-OC(O)NR¹⁵R¹⁵, f) —O—C₁₋₆ alkyl-C(O)NR¹⁵R¹⁵, g) —O—C₁₋₆ alkyl-NR¹⁵C(O)R¹⁵, h) —O—C₁₋₆ alkyl-NR¹⁵C(O)OR¹⁵, i) —O—C₁₋₆ alkyl-NR¹⁵C(O)NR¹⁵R¹⁵, j) —O—C₁₋₆ alkyl-NR¹⁵C(═NH)NR¹⁵R¹⁵, k) —O—C₁₋₆ alkyl-S(O)ₚR¹⁵, l) C₂₋₆ alkenyl-OC(O)R¹⁵, m) —O—C₂₋₆ alkenyl-C(O)OR¹⁵, n) —O—C₂₋₆ alkenyl-OC(O)NR¹⁵R¹⁵, o) —O—C₂₋₆ alkenyl-C(O)NR¹⁵R¹⁵, p) —O—C₂₋₆ alkenyl-NR¹⁵C(O)R¹⁵, q) —O—C₂₋₆ alkenyl-NR¹⁵C(O)OR¹⁵, r) —O—C₂₋₆ alkenyl-NR¹⁵C(O)NR¹⁵R¹⁵, s) —O—C₂₋₆ alkenyl-NR¹⁵C(═NH)NR¹⁵R¹⁵, t) —O—C₂₋₆ alkenyl-S(O)ₚR¹⁵, u) —O—C₂₋₆ alkynyl-OC(O)R¹⁵, v) —O—C₂₋₆ alkynyl-OC(O)OR¹⁵, w) —O—C₂₋₆ alkynyl-OC(O)NR¹⁵R¹⁵, x) —O—C₂₋₆ alkynyl-C(O)NR¹⁵R¹⁵, y) —O—C₂₋₆ alkynyl-NR¹⁵C(O)R¹⁵, z) —O—C₂₋₆ alkynyl-NR¹⁵C(O)OR¹⁵, aa) —O—C₂₋₆ alkynyl-NR¹⁵C(O)NR¹⁵R¹⁵, bb) —O—C₂₋₆ alkynyl-NR¹⁵C(═NH)NR¹⁵R¹⁵, cc) —C₂₋₆ alkynyl-S(O)ₚR¹⁵; and dd) —NR¹⁵R¹⁵;
alternatively, two R¹⁸ groups taken together form ═O, ═NOR¹⁵, or ═NNR¹⁵R¹⁵;
R¹⁹ is R¹²;
R²⁰ is selected from the group consisting of:
a) R¹³, b) F, c) Cl, d) Br, e) I, f) CN, g) NO₂, and h) —OR¹¹;
alternatively, R¹⁹ and R²⁰ taken together are —O(CH₂)ᵤO—;
R²¹, at each occurrence, independently is selected from the group consisting of:
a) H, b) F, c) Cl, d) Br, e) I, f) CN, g) —OR¹¹, h) —NO₂, i) —NR¹¹R¹¹, j) C₁₋₆ alkyl, k) C₁₋₆ acyl, and l) C₁₋₆ alkoxy;
R²² is selected from the group consisting of:
a) C₁₋₆ alkyl, b) C₂₋₆ alkenyl, c) C₂₋₆ alkynyl, d) C₁₋₆ acyl, e) C₁₋₆ alkoxy, f) C₁₋₆ alkylthio, g) saturated, unsaturated, or aromatic C₅₋₁₀ carbocycle, h) saturated, unsaturated, or aromatic 5-10 membered heterocycle containing one or more heteroatoms selected from the group consisting of nitrogen, oxygen, and sulfur, i) —O—C₁₋₆ alkyl-saturated, unsaturated, or aromatic 5-10 membered heterocycle containing one or more heteroatoms selected from the group consisting of nitrogen, oxygen, and sulfur, j) —NR¹¹-C₁₋₆ alkyl-saturated, unsaturated, or aromatic 5-10 membered heterocycle containing one or more heteroatoms selected from the group consisting of nitrogen, oxygen, and sulfur, k) saturated, unsaturated, or aromatic 10-membered bicyclic ring system optionally containing one or more heteroatoms selected from the group consisting of nitrogen, oxygen, and sulfur, l) saturated, unsaturated, or aromatic 13-membered tricyclic ring system optionally containing one or more heteroatoms selected from the group consisting of nitrogen, oxygen, and sulfur, m) —OR¹¹, n) —NR¹¹R¹¹, o) —S(O)ₚR¹¹, and p) —R²¹,
wherein any of a)-l) optionally is substituted with one or more R¹² groups;
alternatively, R²² and one R²¹ group, taken together with the atoms to which they are bonded, form a 5-7 membered saturated or unsaturated carbocycle, optionally substituted with one or more R¹² groups; or a 5-7 membered saturated or unsaturated heterocycle containing one or more atoms selected from the group consisting of nitrogen, oxygen, and sulfur, and optionally substituted with one or more R¹² groups;
R²³ at each occurrence, independently is selected from the group consisting of:
a) hydrogen, b) an electron-withdrawing group, c) aryl, d) substituted aryl, e) heteroaryl, f) substituted heteroaryl, and g) C₁₋₆ alkyl, optionally substituted with one or more R¹² groups;
alternatively, any R²³ and any R²⁰, taken together with the atoms to which they are bonded, form a 5-7 membered saturated or unsaturated carbocycle, optionally substituted with one or more R¹² groups; or a 5-7 membered saturated or unsaturated heterocycle containing one or more atoms selected from the group consisting of nitrogen, oxygen, and sulfur, and optionally substituted with one or more R¹² groups;
p, at each occurrence, is selected from the group consisting of 0, 1, and 2;
r, at each occurrence, is selected from the group consisting of 0, 1, and 2;
t, at each occurrence, is selected from the group consisting of 0, 1, and 2; and
u, at each occurrence, is selected from the group consisting of 1, 2, 3, and 4.

2. A compound of claim 1 selected from the group consisting of: 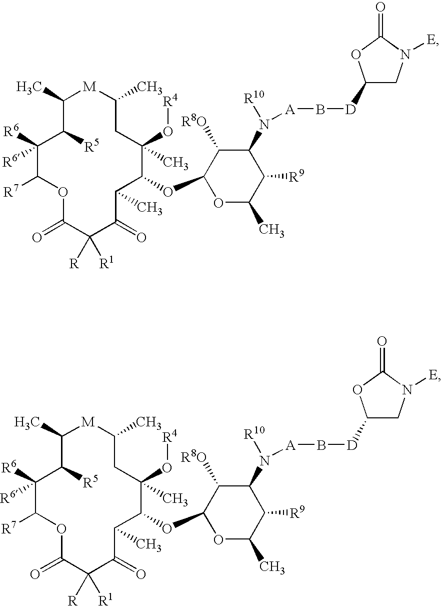 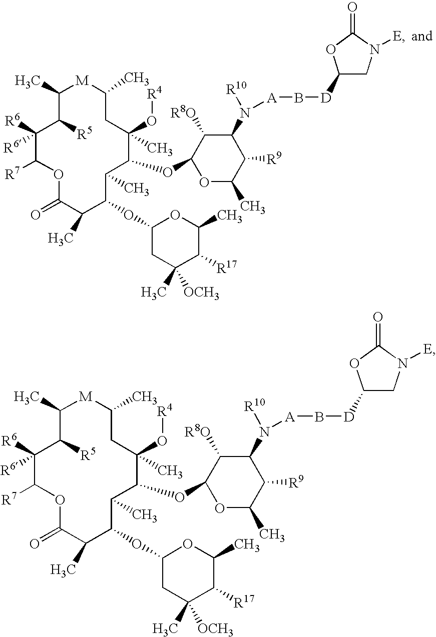
or a pharmaceutically acceptable salt, ester, or prodrug thereof,
wherein A, B, D, E, M, R, R¹, R⁴, R⁵, R⁶, R^{6′}, R⁷, R⁸, R⁹, R¹⁰ and R¹⁷ are as defined in claim 1.

3. A compound of claim 1, selected from the group consisting of: 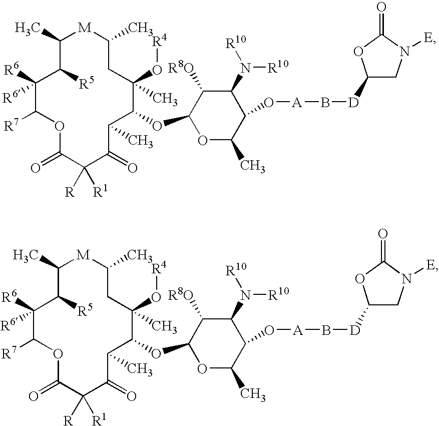 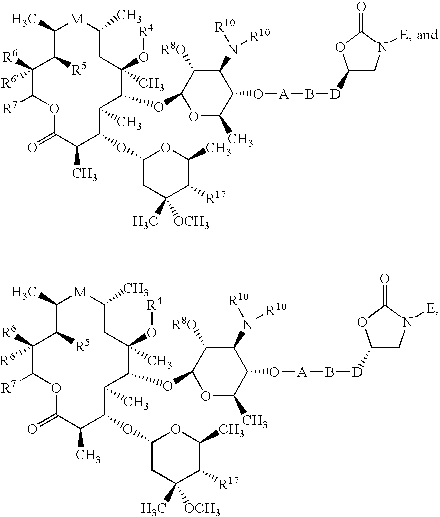
or a pharmaceutically acceptable salt, ester, or prodrug thereof,
wherein A, B, D, E, M, R, R¹, R⁴, R⁵, R⁶, R^{6′}, R⁷, R⁸, R⁹, R¹⁰ and R¹⁷ are as defined in claim 1.

4. A compound of claim 1, selected from the group consisting of: 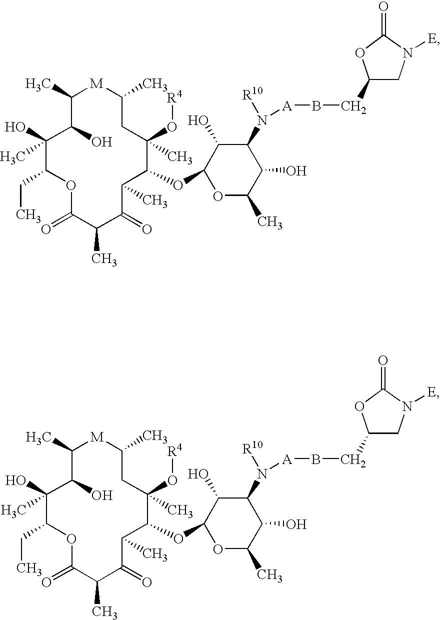 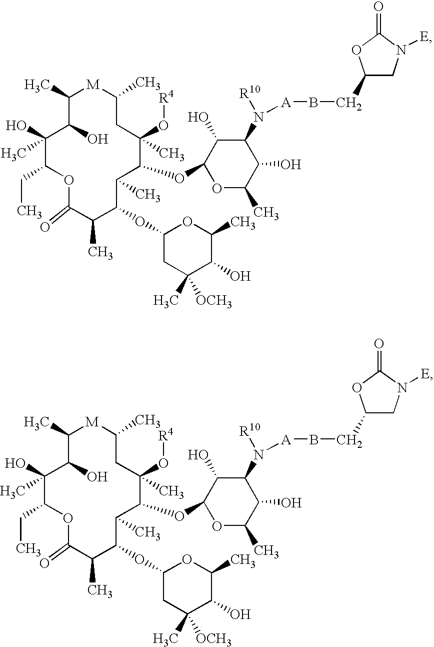 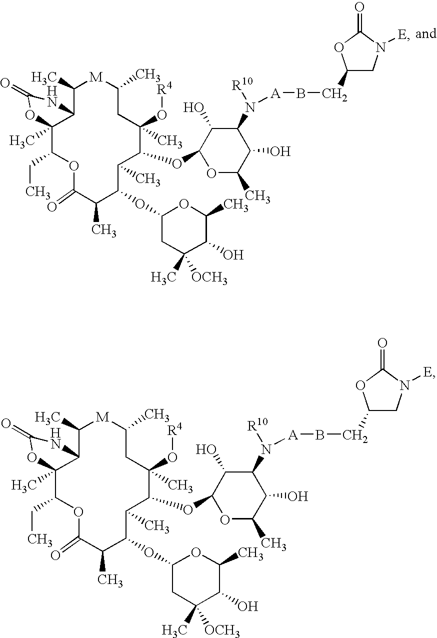
or a pharmaceutically acceptable salt, ester, or prodrug thereof,
wherein A, B, E, M, R⁴, and R¹⁰ are as defined in claim 1.

5. A compound of claim 1, selected from the group consisting of: 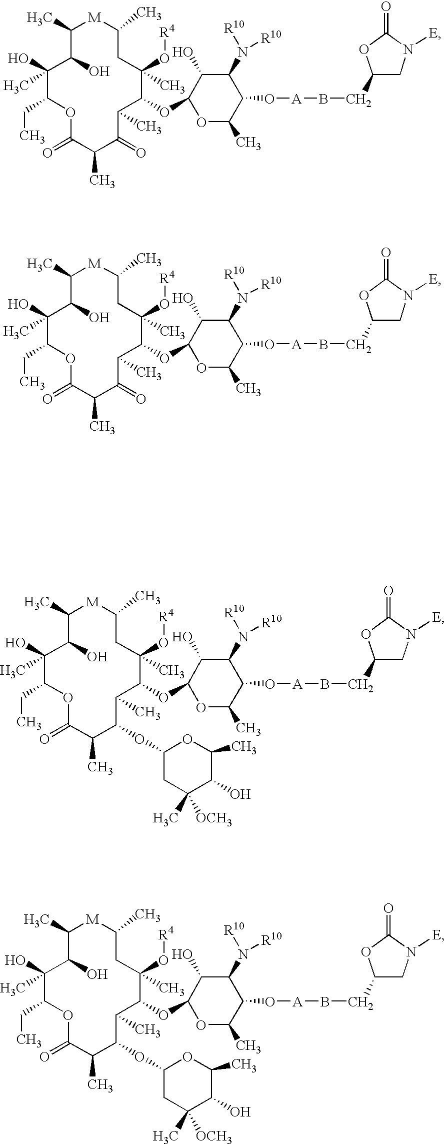 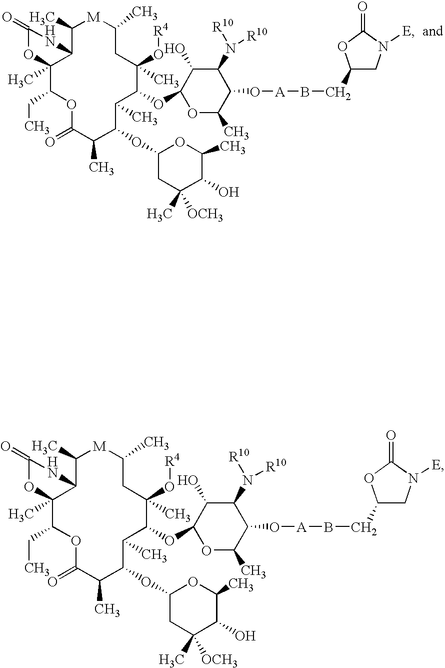
or a pharmaceutically acceptable salt, ester, or prodrug thereof,
wherein A, B, E, M, R⁴, and R¹⁰ are as defined in claim 1.

6. A compound of claim 1, selected from the group consisting of: 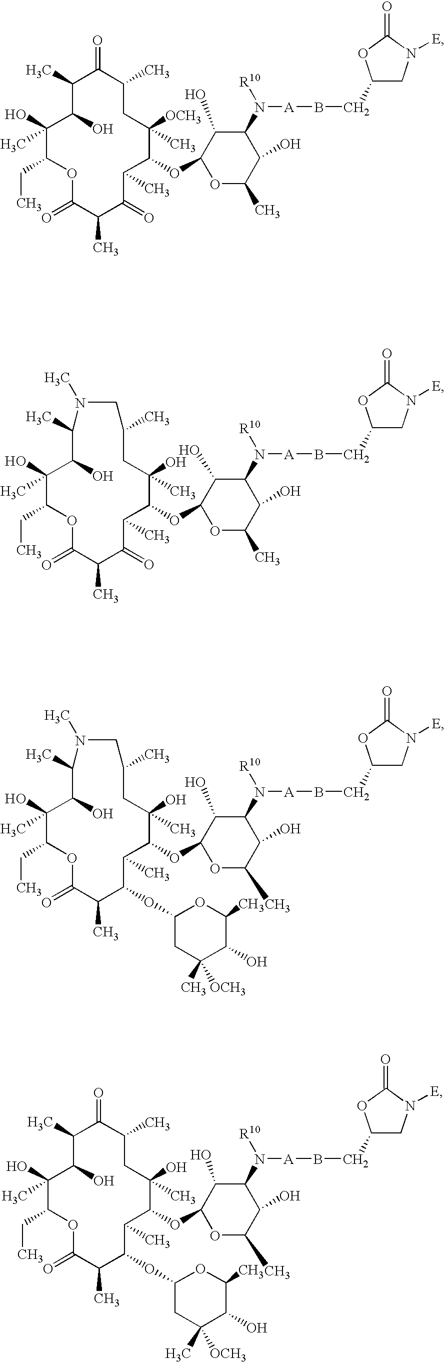 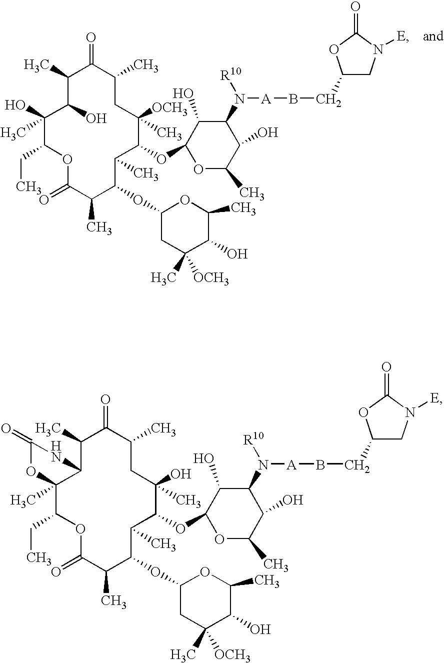
or a pharmaceutically acceptable salt, ester, or prodrug thereof,
wherein A, B, E, and R¹⁰ are as defined in claim 1.

7. A compound of claim 1, selected from the group consisting of: 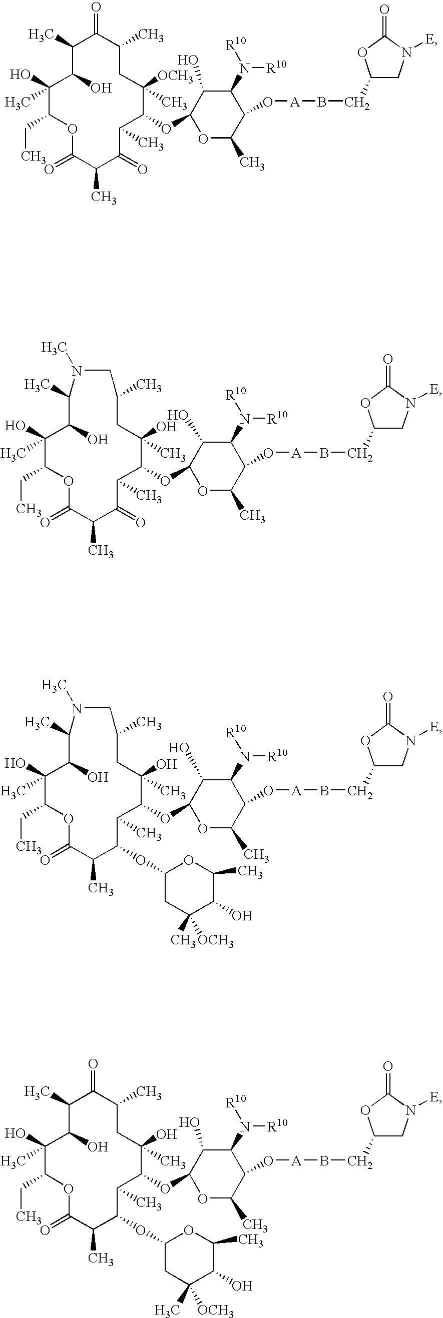 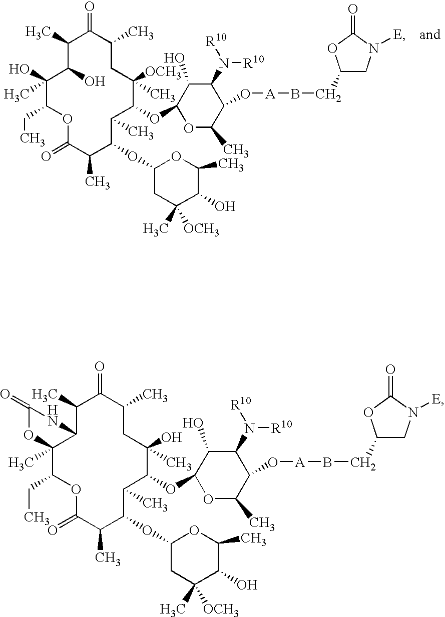
or a pharmaceutically acceptable salt, ester, or prodrug thereof,
wherein A, B, E, and R¹⁰ are as defined in claim 1.

8. A compound according to claim 1, wherein B is selected from the group consisting of:
a) 3-4 membered saturated or unsaturated heterocycle containing one or more heteroatoms selected from the group consisting of nitrogen, oxygen, and sulfur,
b) 6-7 membered saturated, unsaturated, or aromatic heterocycle containing one or more heteroatoms selected from the group consisting of nitrogen, oxygen, and sulfur, and c) 3-4 membered saturated 3-4 membered saturated, unsaturated, or aromatic carbocycle, d) 6-7 membered saturated, unsaturated, or aromatic carbocycle,
wherein any of a)-d) optionally is substituted with one or more R¹² groups.

9. A compound according to claim 1, wherein B is selected from the group consisting of:

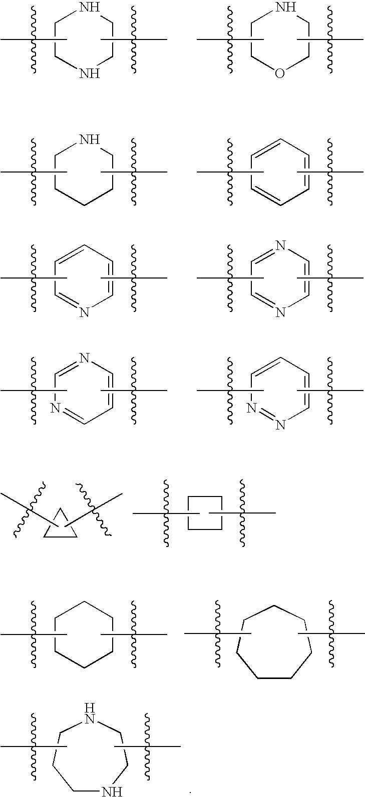

10. The compound according to claim 1, wherein A-B-D is:

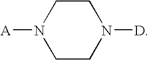

11. The compound according to claim 1, wherein A-B-D is:

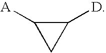

12. A compound having the formula
M—(CH₂)ₘ—B—O
or a pharmaceutically acceptable salt, ester, or prodrug thereof,
wherein M is a macrolide selected from the group consisting of: 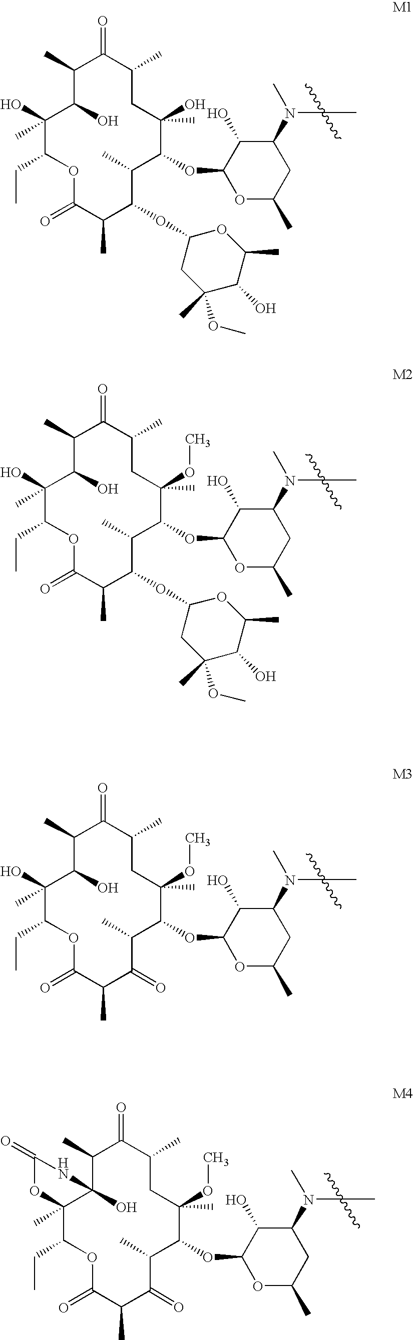 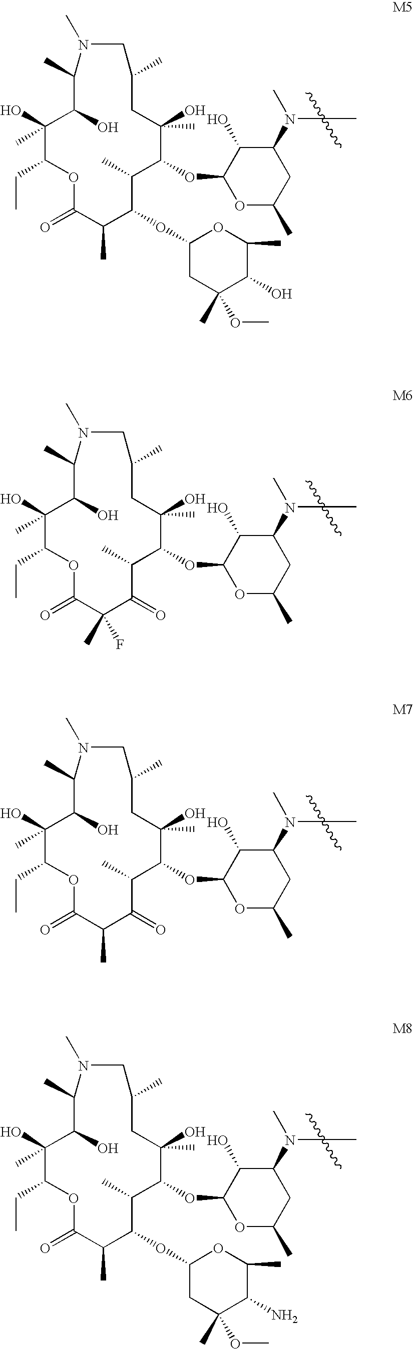 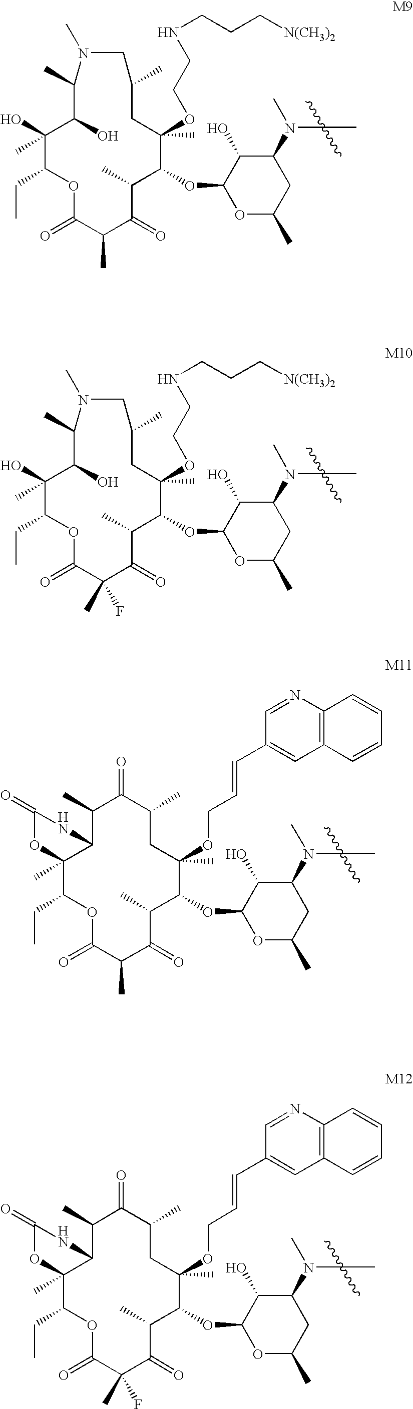 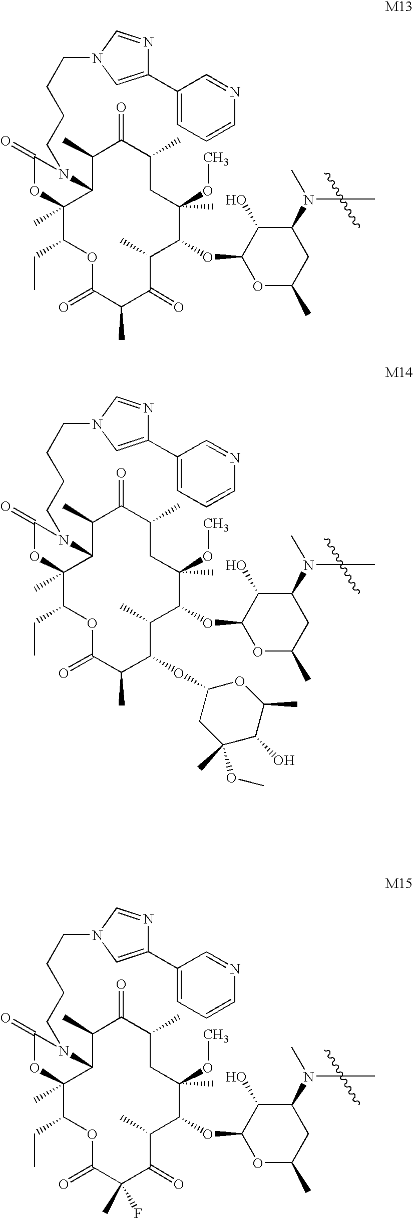 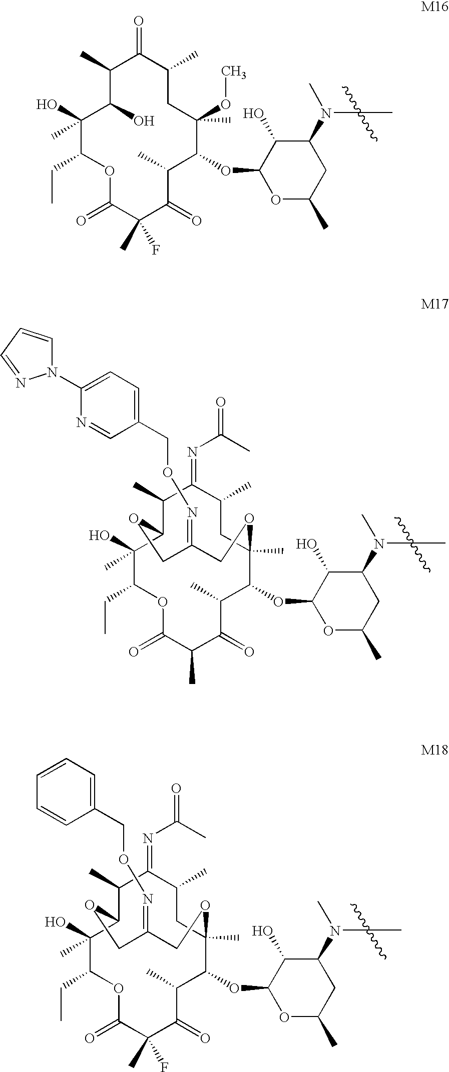 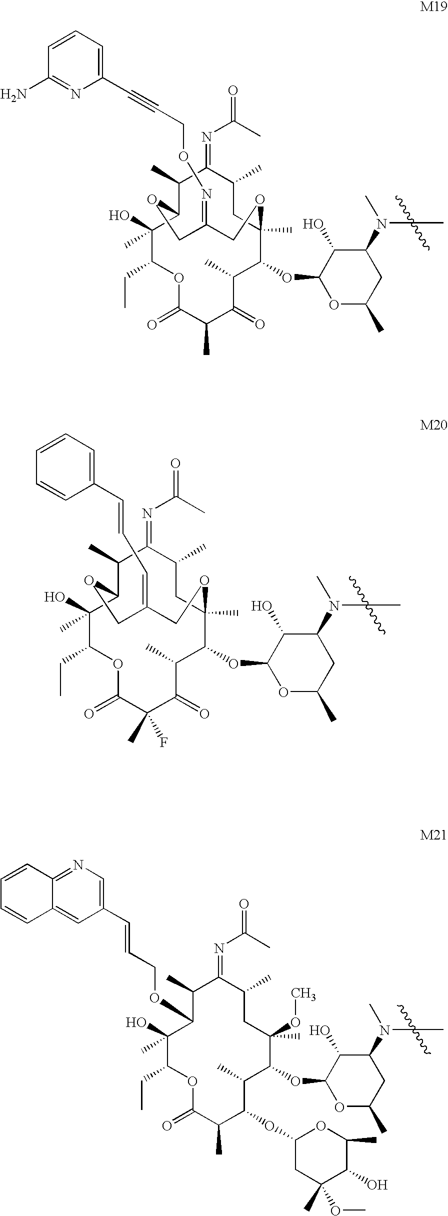 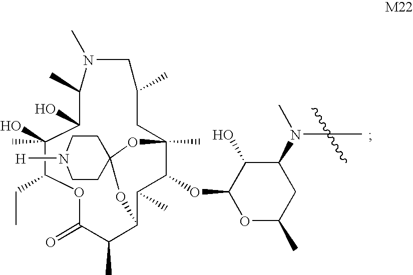
B is a linker selected from the group consisting of: 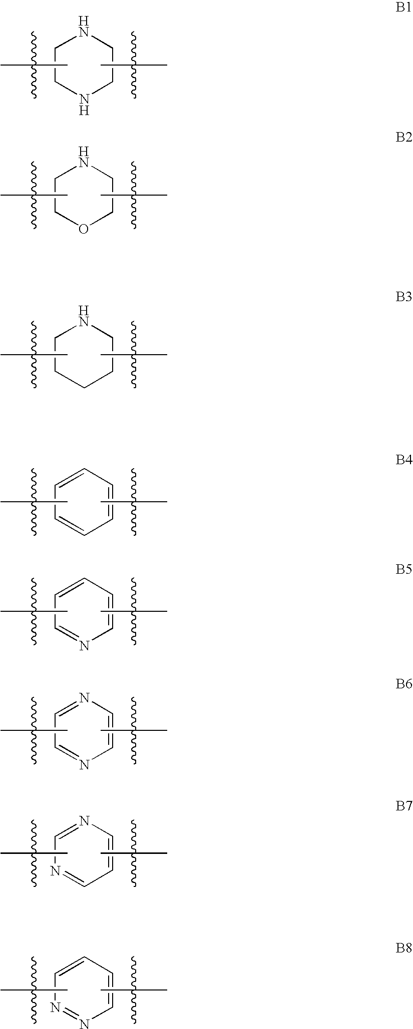 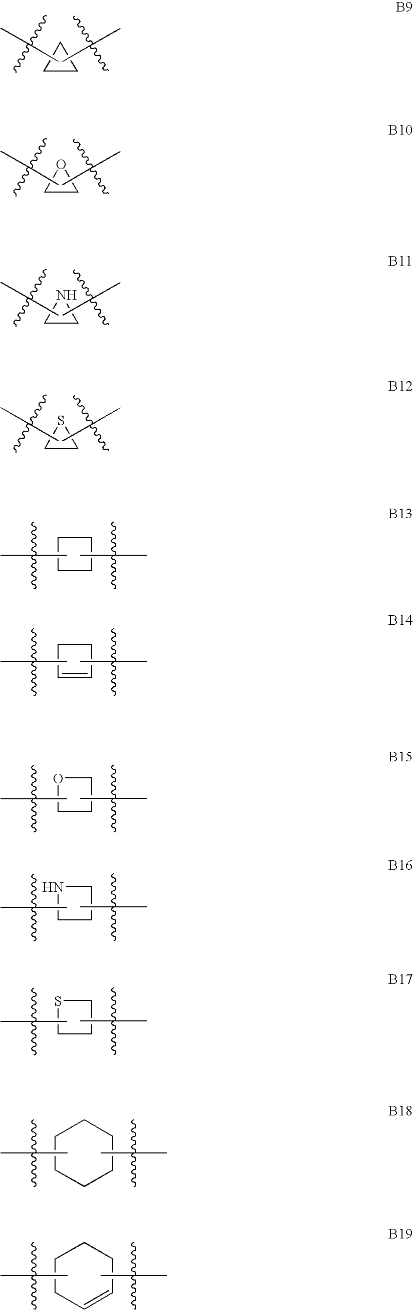 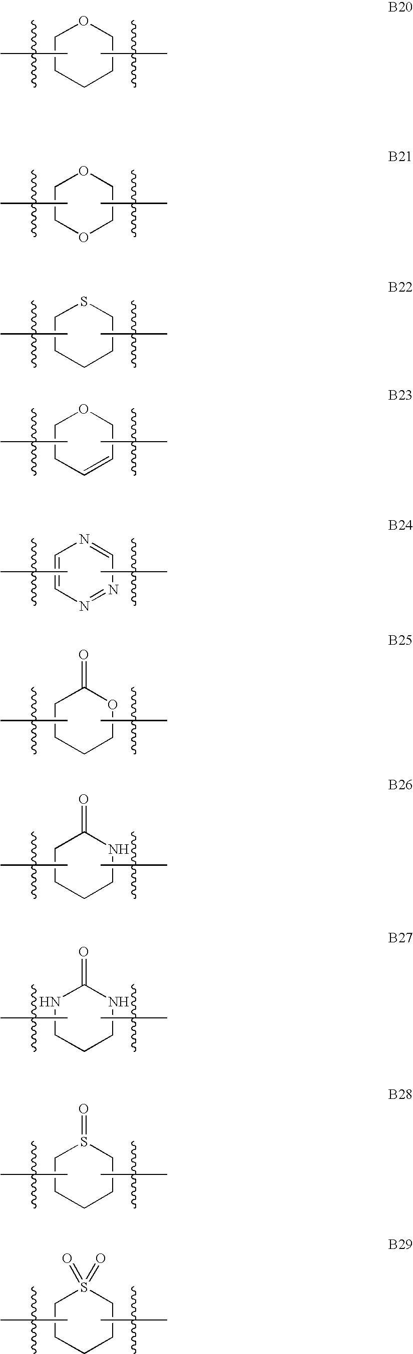 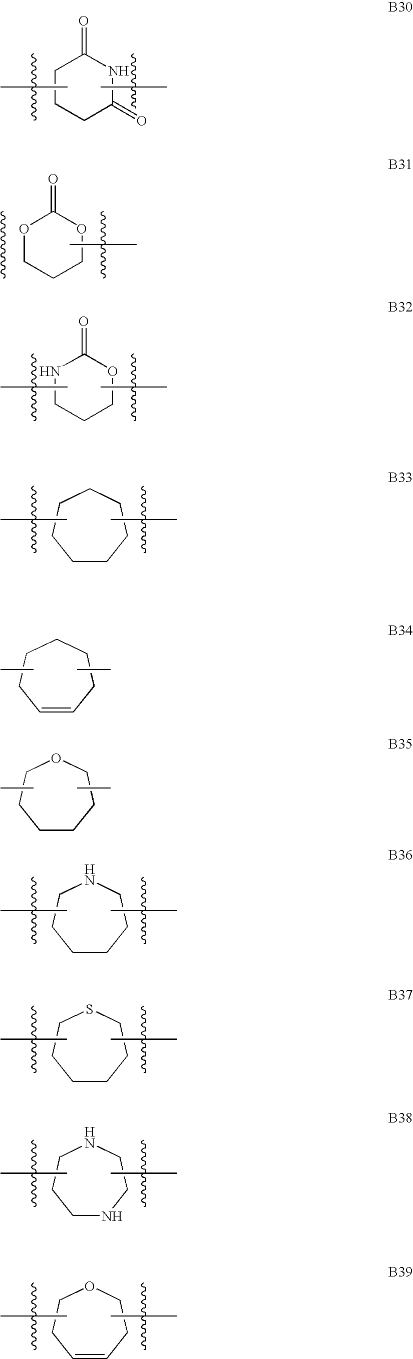 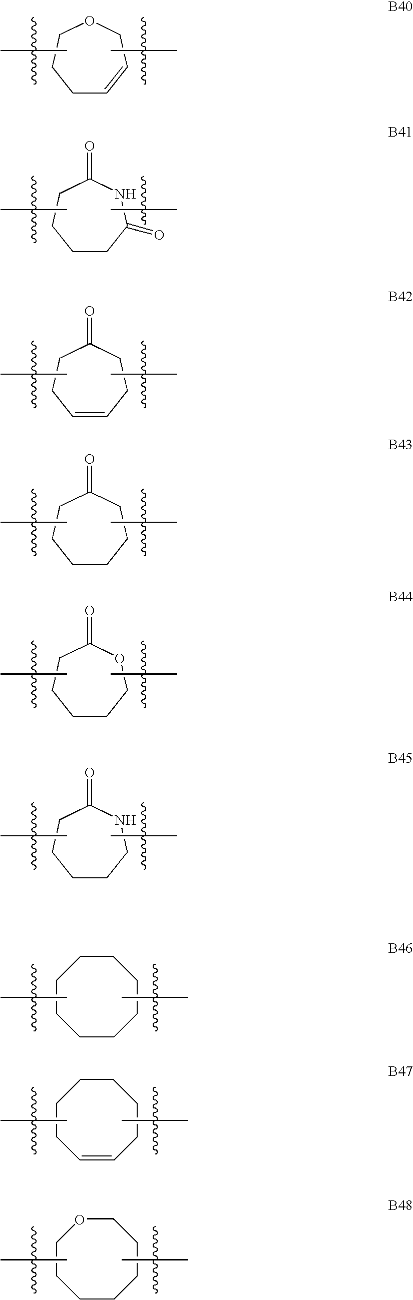 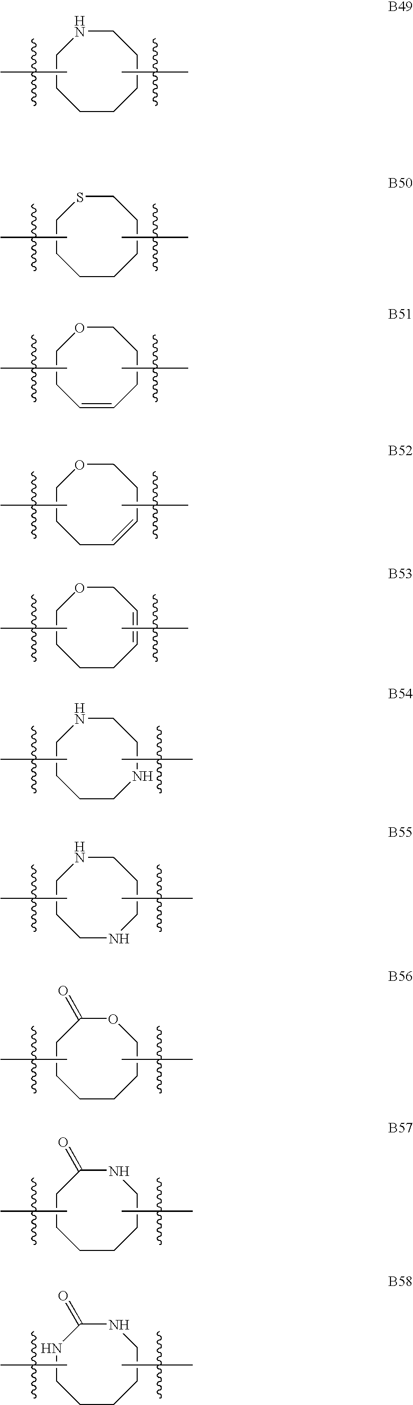 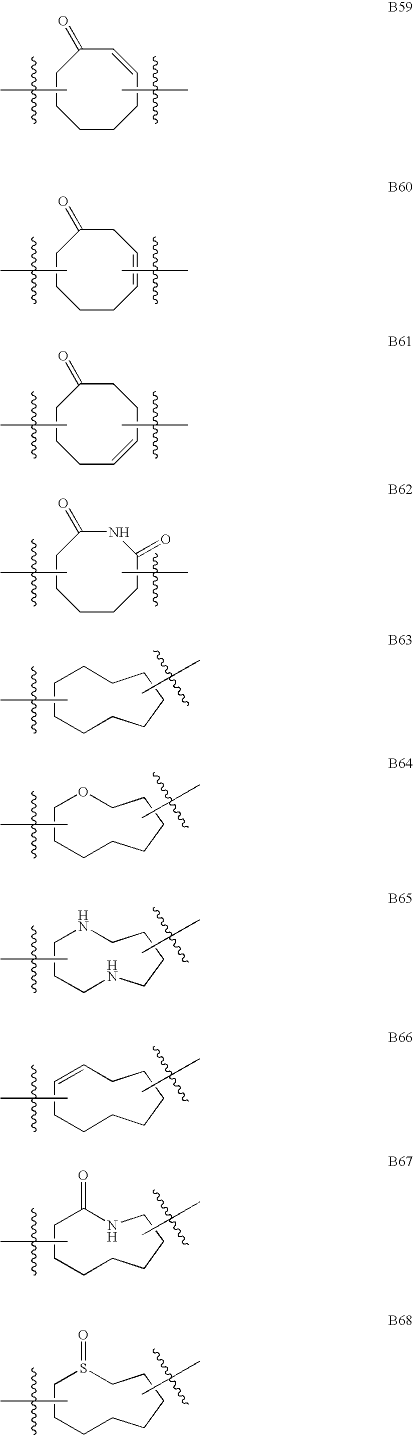 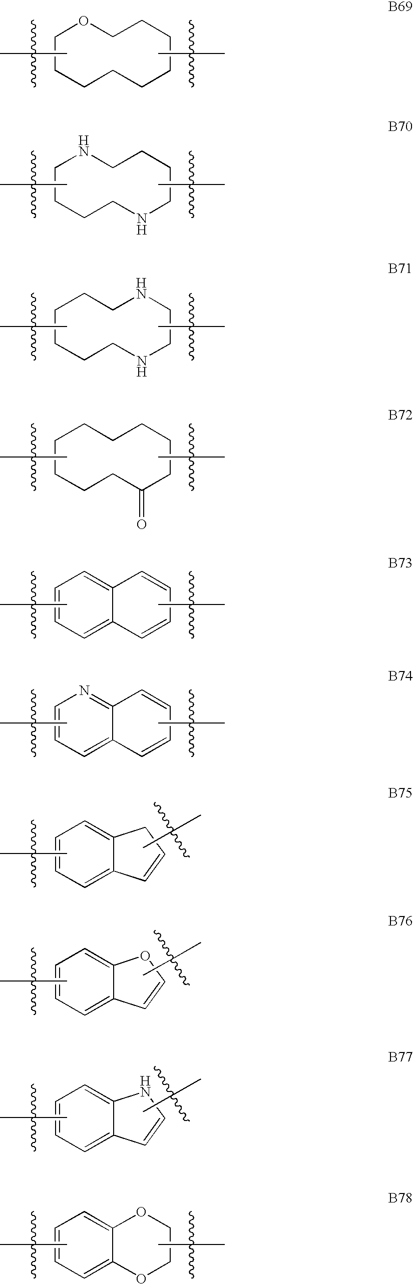 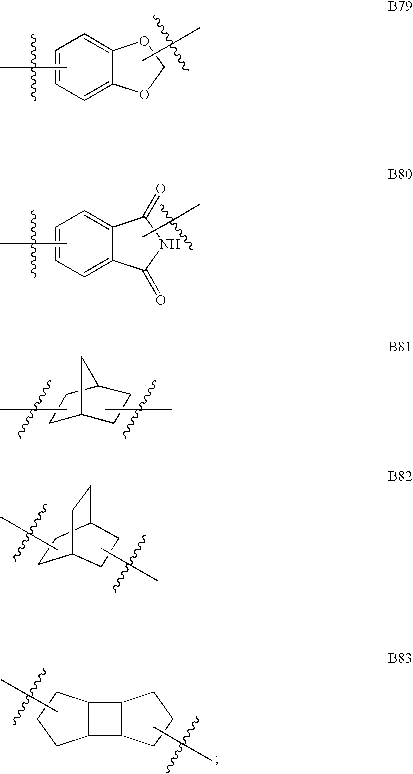
O is a heterocyclic side chain selected from the group consisting of: 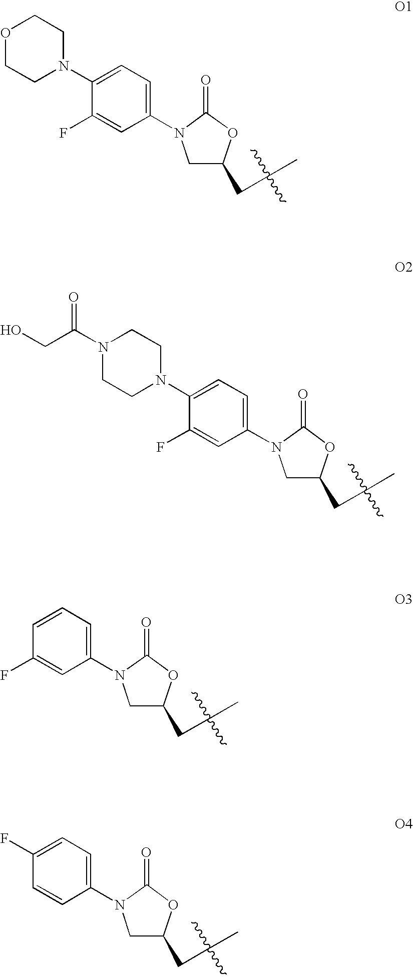 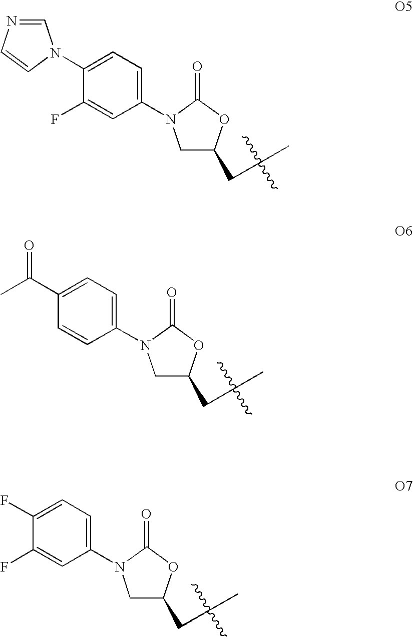 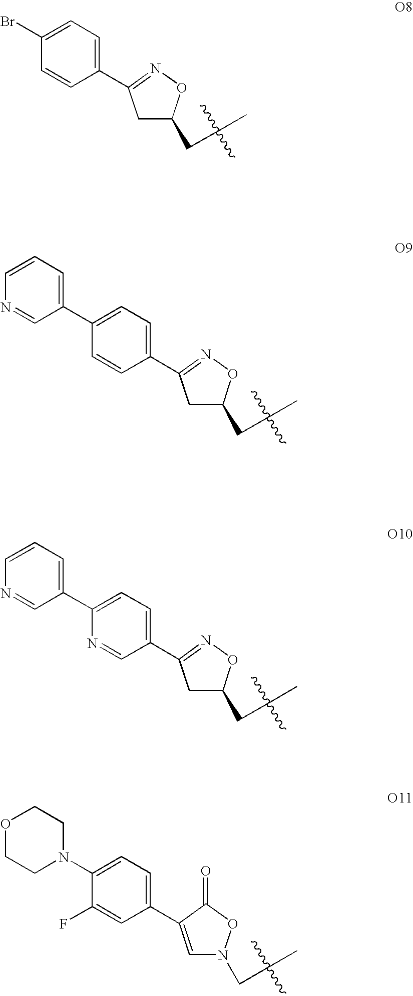 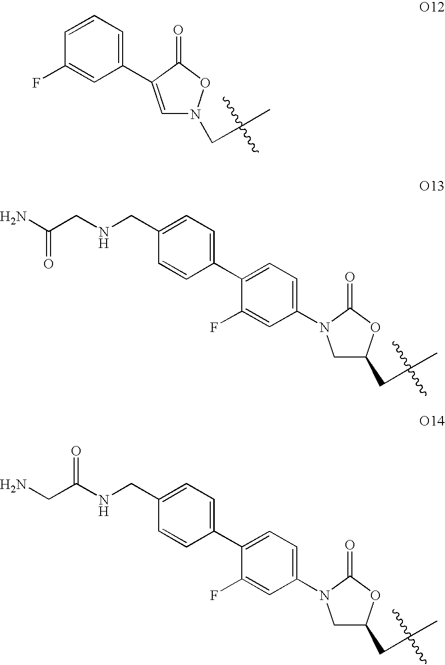 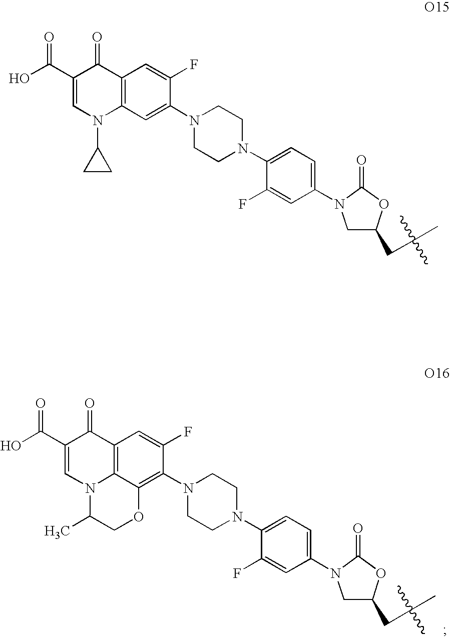
and m is an integer from 1-4.

13. A compound having the formula: 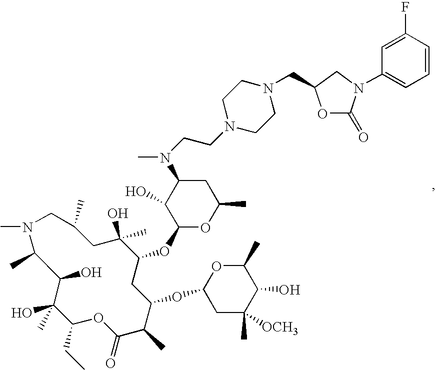
or a pharmaceutically acceptable salt, ester, or prodrug thereof.

14. A compound having the formula: 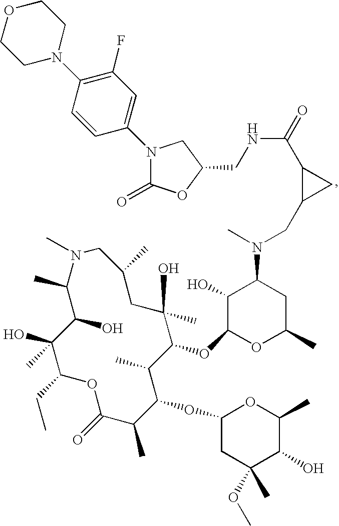
or a pharmaceutically acceptable salt, ester, or prodrug thereof.

15. A pharmaceutical composition comprising a compound according to claim 1 and a pharmaceutically acceptable carrier.

16. A method of treating a disorder selected from a microbial infection a fungal infection, a parasitic disease, a proliferative disease, a viral infection, an inflammatory disease, and a gastrointestinal motility disorder in a mammal comprising administering to the mammal an effective amount of a compound according to claim 1.

17-22. (canceled)

23. The method according to claim 16, wherein the compound is administered orally, parentally, or topically.

24. A method of synthesizing a compound according to claim 1.

25. A medical device containing a compound according to claim 1.

26. The medical device according to claim 25, wherein the device is a stent.
